SEQUENCE LISTING (60 sequences)

SEQ ID NO: 1 - RNA, 21 nt, Artificial Sequence
  mRNA
cugacggugugggugaggagc

SEQ ID NO: 2 - RNA, 23 nt, Artificial Sequence
  IRES element of HCV RNA
ugguaucuagugaggggacacuc

SEQ ID NO: 3 - RNA, 142 nt, Artificial Sequence
  RNA Lasso Probe
gggcagccguccucguccguaugacgagagaagcugaccagagaaacacacgacguaagucgugguacauuaccugguaacugguaucuagugaggggacacucgagaauaacaacaacaacaacaaccagccguccucguc

SEQ ID NO: 4 - RNA, 140 nt, Artificial Sequence
  RNA Lasso Probe
gggcagccguccucguccguaugacgagagaagcugaccagagaaacacacgacguaagucgugguacauuaccugguaaccugacggugugggugaggagcgagaauaacaacaacaacaacaaccagccguccucguc

SEQ ID NO: 5 - DNA, 23 bp, Artificial Sequence
  HCV-Oligo-23 DNA Probe
ctcacaggggagtgatctatggt

SEQ ID NO: 6 - DNA, 61 bp, Artificial Sequence
  HCV-oligo-61 DNA Probe
gcgtgaagacagtagttcctcacaggggagtgatctatggtggagtgtcgcccccaatcgg

SEQ ID NO: 7 - RNA, 151 nt, Artificial Sequence
  RNA anti-TNF Lasso TNF4-DB
gggcagccguccucguccguaugacgagagaagcugaccagagaaacacacgacguaagucgugguacauuaccugguaaccugacggugugggugaggagcgagaauaacaaccuccucacaacaacaacaacaaccagccguccucguc

SEQ ID NO: 8 - RNA, 144 nt, Artificial Sequence
  RNA anti-TNF Lasso TNF4-MB9
gggcagccguccucguccguaugacgagagaagcugaccagagaaacacacgacguaagucgugguacauuaccugguaacaacagucguugaucugacggugugggugaggagcaucaacgacaacaaccagccguccucguc

SEQ ID NO: 9 - DNA, 21 bp, Artificial Sequence
  TNF-DNAoligo 21
ctgacggtgtgggtgaggagc

SEQ ID NO: 10 - DNA, 61 bp, Artificial Sequence
  TNF-DNAoligo 61
gagatagcaaatcggctgacggtgtgggtgaggagcacgtagtcggggcagccttgtccct

SEQ ID NO: 11 - RNA, 141 nt, Artificial Sequence
  RNA lasso HCVgt1b
gggcagccguccucguccguaugacgagagaagcugaccagagaaacacacgacguaagucgugguacauuaccugguaaccauugagcggguuuauccaagagagaauaacaacaacaacaacaaccagccguccucguc

SEQ ID NO: 12 - RNA, 140 nt, Artificial Sequence
  RNA lasso HCVgt2a
gggcagccguccucguccguaugacgagagaagcugaccagagaaacacacgacguaagucgugguacauuaccugguaacggccgggcauagaguggguuugagaauaacaacaacaacaacaaccagccguccucguc

SEQ ID NO: 13 - RNA, 140 nt, Artificial Sequence
  RNA lasso HCVgt3a
gggcagccguccucguccguaugacgagagaagcugaccagagaaacacacgacguaagucgugguacauuaccugguaacuucuggguauugagcggguuggagaauaacaacaacaacaacaaccagccguccucguc

SEQ ID NO: 14 - RNA, 142 nt, Artificial Sequence
  RNA lasso HCVgt5a
gggcagccguccucguccguaugacgagagaagcugaccagagaaacacacgacguaagucgugguacauuaccugguaacccgggcauugagcggguuaauccgagaauaacaacaacaacaacaaccagccguccucguc

SEQ ID NO: 15 - RNA, 141 nt, Artificial Sequence
  RNA lasso HCVgt6b
gggcagccguccucguccguaugacgagagaagcugaccagagaaacacacgacguaagucgugguacauuaccugguaaccuccaggcauugagcggguuuggagaauaacaacaacaacaacaaccagccguccucguc

SEQ ID NO: 16 - RNA, 115 nt, Artificial Sequence
  NP1 Lasso Probe
gggcagccguccucguccguaugacgagagaagcugaccagagaaacacacgacguaagucgugguacauuaccuggucugacggugugggugaggagcaccagccguccugcuc

SEQ ID NO: 17 - DNA, 103 bp, Artificial Sequence
  NP2 Lasso Probe
gggaccacgacttacgtcgtgtgtttctctggtcagcttctctcgtcatacggacgaggacggctgcugacggugugggugaggagcaccagccguccugcuc

SEQ ID NO: 18 - RNA, 32 nt, Artificial Sequence
  NP3 Lasso Probe
gcugaucugacggugugggugaggagccagcc

SEQ ID NO: 19 - RNA, 72 nt, Artificial Sequence
  NP4 Lasso Probe
gggaacaacaacagucguugaucugacggugugggugaggagcaucaacgacaacaaccagccguccugcuc

SEQ ID NO: 20 - RNA, 127 nt, Artificial Sequence
  PCR2 Lasso Probe
gggcguccucguccguaugacgagagaagcugaccagagaaacacacgacguaagucgugguacauuaccugguaacaacagucguugaucugacggugugggugaggagcaucaacgacaacaacc

SEQ ID NO: 21 - RNA, 98 nt, Artificial Sequence
  PCR3 Lasso Probe
gggaccagagaaacacacgacguaagucgugguacauuaccugguaacaacagucguugaucugacggugugggugaggagcaucaacgacaacaacc

SEQ ID NO: 22 - RNA, 106 nt, Artificial Sequence
  NP5 Lasso Probe
gggcagccguccucguccguaugacgagagaagcugauaacaacaacagucguugaucugacggugugggugaggagcaucaacgacaacaaccagccguccucgu

SEQ ID NO: 23 - RNA, 107 nt, Artificial Sequence
  NP6 Lasso Probe
gggcagccguccucguccguaugacgagagaagcugauaacaacaacaacaacaacacugacggugugggugaggagcaacaacaacaacaaccagccguccucguc

SEQ ID NO: 24 - RNA, 150 nt, Artificial Sequence
  NP7 RNA Probe
gggcacgaauccuaaaccucacuuccugcaugagguuuaggauucgugcaacaacaaccugacggugugggugaggagcaacaacaacgaguugaauuaucagcuuacuuccugcauaagcugauaauucaacucaaccauaugggaucc

SEQ ID NO: 25 - RNA, 92 nt, Artificial Sequence
  RNA Lasso Probe
gggcagccguccucguccguaugacgagagaagcugaccagagaaacacacgacguaagucgugguacauuaccuggucagccguccucguc

SEQ ID NO: 26 - RNA, 75 nt, Artificial Sequence
  RNA Lasso Probe
guccucguccguaugacgagagaagcugaccagagaaacacacgacguaagucgugguacauuaccuggucagcc

SEQ ID NO: 27 - RNA, 117 nt, Artificial Sequence
  RNA Lasso Probe
guccugugcguaugacagagaagucaaccagagaaacacacguugugguauauuaccuggucaagaaguucccuguuccuuuuuuucccgagccuugucccuugaagagaacugacc

SEQ ID NO: 28 - RNA, 21 nt, Artificial Sequence
  MuTNF mRNA
guucucuucaagggacaagcu

SEQ ID NO: 29 - RNA, 122 nt, Artificial Sequence
  RNA lasso probe
guccucguccguaugacgagagaagcugaccagagaaacacacgacguaagucgugguacauuaccugguaacagaggcugagacauaggcacaacaacaacaacaacaacaacacucagcc

SEQ ID NO: 30 - RNA, 20 nt, Artificial Sequence
  MuTNF mRNA
gugccuaugucucagccucu

SEQ ID NO: 31 - RNA, 119 nt, Artificial Sequence
  mRNA Lasso Probe
gggcagccguccucguccguaugacgagagaagcugaccagagaaacacacgacguaagucgugguacauuaccugguaacgagaauaacaacaacaacaacaaccagccguccucguc

SEQ ID NO: 32 - RNA, 102 nt, Artificial Sequence
  mRNA Lasso Probe
guccucguccguaugacgagagaagcugaccagagaaacacacgacguaagucgugguacauuaccugguaacgagaauaacaacaacaacaacaaccagcc

SEQ ID NO: 33 - RNA, 140 nt, Artificial Sequence
  RNA Lasso Probe TNF4-DB unprocessed
gggcagccguccucguccguaugacgagagaagcugaccagagaaacacacgacguaagucgugguacauuaccugguaaccugacguguggguagaggacggagaauaacaacaacaacaacaaccagccguccucguc

SEQ ID NO: 34 - RNA, 149 nt, Artificial Sequence
  RNA Lasso Probe TNF4
gggcagccguccucguccguaugacgagagaagcugaccagagaaacacacgacguaagucgugguacauuaccugguaaccugacguguggguagaggacggagaauaacaaccuccuccacaacaacaacaaccagccguccucguc

SEQ ID NO: 35 - RNA, 21 nt, Artificial Sequence
  TNF4 Lasso target 1
cugacggugugggugaggagc

SEQ ID NO: 36 - RNA, 21 nt, Artificial Sequence
  TNF4 Lasso target 2
gacugccacacccacuccucg

SEQ ID NO: 37 - RNA, 21 nt, Artificial Sequence
  TNF4 Lasso target 3
gacugccacacccacucgucg

SEQ ID NO: 38 - RNA, 21 nt, Artificial Sequence
  TNF4 Lasso target 4
gacugccacaccgacucgucg

SEQ ID NO: 39 - RNA, 21 nt, Artificial Sequence
  TNF4 Lasso target 5
gacugccacacccacuggucg

SEQ ID NO: 40 - RNA, 21 nt, Artificial Sequence
  TNF4 Lasso target 6
gacugccacaccgacuggucg

SEQ ID NO: 41 - RNA, 147 nt, Artificial Sequence
  RNA Lasso Probe TNF4-MB9
gggcagccguccucguccguaugacgagagaagcugaccagagaaacacacgacguaagucgugguacauuaccugguaacaacaacagucguugaucugacggugugggugaggagcaucaacgacaacaaccagccguccucguc

SEQ ID NO: 42 - RNA, 21 nt, Artificial Sequence
  TNF Specificity Target 1
cugacggugugggugaggagc

SEQ ID NO: 43 - RNA, 21 nt, Artificial Sequence
  TNF Specificity Target 2
gcuccucacccacaccgucag

SEQ ID NO: 44 - RNA, 21 nt, Artificial Sequence
  TNF Specificity Target 3
gcugcucacccacaccgucag

SEQ ID NO: 45 - RNA, 21 nt, Artificial Sequence
  TNF Specificity Target 4
gcugcucagccacaccgucag

SEQ ID NO: 46 - RNA, 21 nt, Artificial Sequence
  TNF Specificity Target 5
gcuggucacccacaccgucag

SEQ ID NO: 47 - RNA, 21 nt, Artificial Sequence
  TNF Specificity Target 6
gcuggucagccacaccgucag

SEQ ID NO: 48 - RNA, 29 nt, Artificial Sequence
  HCV IRES genotype 1b
ucuuggaucaacccgcucaaugccuggag

SEQ ID NO: 49 - RNA, 29 nt, Artificial Sequence
  HCV IRES genotype 1a
ucuuggauaaacccgcucaaugccuggag

SEQ ID NO: 50 - RNA, 29 nt, Artificial Sequence
  HCV IRES genotype 2a
ucuuggauaaacccacucuaugcccggcc

SEQ ID NO: 51 - RNA, 29 nt, Artificial Sequence
  HCV IRES genotype 3a
ucuuggagcaacccgcucaauacccagaa

SEQ ID NO: 52 - RNA, 29 nt, Artificial Sequence
  HCV IRES genotype 4a
ucuuggauuaacccgcucaaugcccggaa

SEQ ID NO: 53 - RNA, 29 nt, Artificial Sequence
  HCV IRES genotype 5a
ucuuggauaaacccgcucaaugcccggag

SEQ ID NO: 54 - RNA, 32 nt, Artificial Sequence
  HCV IRES genotype 6b
uccauuggaucaaacccgcucaaugccuggag

SEQ ID NO: 55 - RNA, 119 nt, Artificial Sequence; identical to SEQ ID NO: 31
  RNA Lasso Probe

SEQ ID NO: 56 - RNA, 22 nt, Artificial Sequence
  RNA Lasso Probe var. 1
cauugagcggguugauccaaga

SEQ ID NO: 57 - RNA, 21 nt, Artificial Sequence
  RNA Lasso Probe var. 2
ggccgggcauagaguggguuu

SEQ ID NO: 58 - RNA, 21 nt, Artificial Sequence
  RNA Lasso Probe var. 3
uucuggguauugagcggguug

SEQ ID NO: 59 - RNA, 23 nt, Artificial Sequence
  RNA Lasso Probe var. 4
ccgggcauugagcggguuaaucc

SEQ ID NO: 60 - RNA, 22 nt, Artificial Sequence
  RNA Lasso Probe var. 5
cuccaggcauugagcggguuug

(19) United States
(12) Patent Application Publication
(10) Pub. No.: US 2009/0170719 A1
(43) Pub. Date: Jul. 2, 2009

(54) SUPERIOR HYBRIDIZATION PROBES AND METHODS FOR THEIR USE IN DETECTION OF POLYNUCLEOTIDE TARGETS

(76) Inventors: Sergei A. Kazakov, Los Gatos, CA (US); Anne Dallas, Santa Cruz, CA (US); Brian H. Johnston, Scotts Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/328,705

(22) Filed: Dec. 4, 2008

Related U.S. Application Data
(60) Provisional application No. 61/005,505, filed on Dec. 4, 2007.

(51) Int. Cl. C40B 30/04; C12Q 1/68; C07H 21/04

(52) U.S. Cl. 506/9; 435/6; 536/24.3

(57) ABSTRACT

We describe new hybridization probes and methods for their use in detection, identification, and quantitation of polynucleotides such as RNA and DNA. Ordinary short oligonucleotide probes usually provide higher sequence-specificity but lower efficacy of hybridization than longer ordinary polynucleotide probes where both are fully complementary to the target polynucleotide. Our new polynucleotide probes combine the hybridization efficacy of long probes with the sequence-specificity of short probes. The polynucleotide probes contain a target binding domain and a binding enhancer domain, where the binding enhancer domain does not for stable structures under hybridizing conditions with the target binding domain or its corresponding target. These binding enhancer domains are able to improve the hybridization features of the target binding domain as well as the signal-to-noise ratio for target detection. Detection methods based on these probes allow fast, accurate, and sensitive detection of target polynucleotides (either qualitatively or quantitatively) and can be easily multiplexed.

### FIELD OF THE INVENTION

The present invention provides new hybridization probes and methods for their use in a variety of polynucleotide assays, including polynucleotide detection, isolation, identification, quantitation, and the like. They can be used to analyze the expression, stability and the presence of single-nucleotide polymorphisms in polynucleotides including mRNA, cRNA, cDNA, genomic DNA, mitochondrial DNA, microbe RNA, microbe DNA, etc. As such, the compositions and methods of the present invention are useful for research and diagnostic purposes in medicine, agriculture, and biodefense.

### BACKGROUND AND RELATED ART

Testing of experimental drugs that inhibit expression of specific genes (e.g. small interfering RNAs) requires fast, accurate and robust methods for measuring the levels of specific mRNAs present in cells before, during and after treatment. Detection and quantification of RNAs is also indispensable for diagnostics for infectious and genetic diseases as well as for monitoring disease progression and response to therapy. Despite the progress of the last few years, current methods for measuring specific RNA levels in biological specimens still have technical limitations and potential biases (Ding & Cantor 2004). Methods based on target amplification require laborious isolation and purification of cellular RNAs to separate them from DNA and inhibitors of polymerases. Variability of the sequence and secondary structures of RNA targets makes it difficult to identify sets of PCR primer sequences for multiplexed qPCR, where each primer should have the same affinity and specificity for its target as all the others have for their respective targets. Another group of methods, based on signal amplification, can avoid the purification and replication of target sequences and hence is less prone to the biases that can occur during those steps. However, these methods usually rely on slow (e.g. overnight), non-stringent hybridization, which always compromises sequence-specificity for binding efficacy (see below). Moreover, they also are not optimal for multiplexing because uniform hybridization and washing conditions cannot provide unbiased, simultaneous detection of both AT- and GC-rich target sequences. There is hence a need for improved methods that allow fast, sensitive, highly accurate, multiplex RNA quantification through signal amplification.

Nucleic acid probes and primers. Probes and primers designed to bind sequence-specifically to their polynucleotide targets through complementary Watson-Crick base-pairing are usually synthetic oligonucleotides. They can also bind to imperfectly complementary (mismatched) target sequences, but with a reduced affinity compared to perfectly matched partners. The differences in thermostability between a perfect duplex and a mismatched duplex depend on length, GC-content, and sequence as well as the type and position of mismatches.

Most hybridization-based assays use DNA probes or their derivatives. However, RNA hybridization probes, which are commonly used in Northern blots and in situ hybridization assays, are in many cases superior to DNA probes, especially when targeting RNA molecules (Thompson & Gillespie 1987; Flaspohler & Milcarek 1992; Singh et al. 1994; Fonsecca et al. 1996; Huang et al. 1998; Breir, 1999; Bisucci et al. 2000; Certa et al. 2001; Ramkinson et al. 2006). RNA-RNA hybrids are more stable than the corresponding DNA-RNA and DNA-DNA duplexes (Lesnik & Freier 1995; Sugimoto et al. 1995; Wu et al. 2002). RNA probes also have faster hybridization kinetics and a better ability to bind structured targets than corresponding DNA probes (Huang et al. 1998; Majlessi et al. 1998). Finally, the notorious instability of RNA in solution, which is the most common argument against using it, is not a problem even for overnight hybridization reactions as long as divalent metal ions are chelated and RNases are inactivated (Lockhart et al. 1996).

Conditions that favor effective duplex formation also promote intrastrand duplex formation in both target and probes. In this instance, longer probes (≧60 nt) have higher affinity for targets and are usually more effective than shorter probes because they have multiple sites to initiate base pairing with the structured targets (Chou et al. 2004). However, shorter oligonucleotide probes (≦25 nt) are generally more sequence-specific than longer ones because of lower affinity of the short probes to mismatched targets, providing the greatest discrimination between closely related sequences (Monia et al. 1992; Hougaard et al. 1997; Majlessi et al. 1998; Toulme et al. 2001).

The trade-off between high affinity for the target and low sequence-specificity of binding is a major limitation for designing allele-specific and multiplex hybridization probes (as well as RT-PCR) primers targeting sequences with different GC-content (Ratilainen et al. 2000; Toulme et al. 2001). Increasing the affinity of these agents to their intended targets will simultaneously decrease their sequence-specificity (Toulme et al. 2001). Hybridization and primer-extension assays dealing with individual sequences can be optimized for maximum selectivity by adjusting temperature, incubation time, salt, and formamide concentration in the hybridization and washing steps. However, multiplexing assays, in which multiple probe-target hybridizations are conducted simultaneously under the same conditions, lack this customizing option.

There are several ways to design SNP-sensitive hybridization probes and primers. The first approach is to use chemistries that provide tight binding even for short pairing regions. In this way, a single mismatch has a large impact on the helical stability. In the case of Locked Nucleic Acids (LNAs), each substitution of an LNA residue for a DNA residue in a primer sequence increases the melting temperature (Tₘ) by 2-10° C. per LNA monomer (depending on sequence content) when hybridized to RNA targets, including miRNAs (Braasch et al. 2002; Jacobsen et al. 2002; Valoczi et al. 2004; Fluiter et al. 2005).

The second approach is to use cooperative hybridization of two or more short oligonucleotide probes and primers (e.g., 7-12 nucleotides in length) to adjacent target sites. This may be done in two ways: (1) “head-to-tail” or tandem hybridization, in which the complex is stabilized through stacking interactions at the interface between the probes (Wang et al. 2003); and (2) “side-by-side” hybridization of probes that have additional dimerization (“kissing”) sequences at ends that are complementary to each other but not to the target (Maher & Dolnick 1988; Kandimalla et al. 1995). All these probes were originally designed for long RNA targets. In the case of short RNA targets (e.g., miRNAs and other small non-coding RNAs), stem-loop (hairpin-like) probes with short single-stranded overhangs can be designed. Hybridization of single-stranded targets to such probes is enhanced by contiguous stacking interactions between the end of the probe participating in the stem-loop structure (e.g., the 5′-end) and the adjacent end of the probe-hybridized target (e.g., 3′-end), and is highly sequence specific (Walter et al. 1994; Lane et al. 1997; Ricceli et al. 2001; Chen et al. 2005).

The third approach is to use probes and primers with special secondary structures (stringency elements) that can improve mismatch discrimination upon hybridization. As a result of competitive hybridization, the antisense sequence of the probe forms a perfect duplex with the target as the stringency elements dissociate. Targets containing mismatches or deletions form, at best, unstable duplexes even under optimized conditions. Three types of such stringency elements are commonly used. Type A comprises a separate masking oligonucleotide strand that is complementary to a part of the antisense sequence. The chemistry (DNA, RNA or derivatives thereof), length, and location of the masking oligonucleotide depend on the sequence of the target site (Vary 1987; Li et al. 2002). Type B comprises terminal hairpin structures that are complementary to one or both ends of the antisense sequence (Roberts & Crothers 1991; Hertel et al. 1998; Ohmichi & Kool 2000). Type C comprises short “arms” flanking the antisense sequence at both ends. The sequences of the arms are complementary to each other but not to the antisense sequence. In the absence of target, such probes, also known as “molecular beacons”, form stem-loop structures in which the antisense sequence is located in the loop (Tyagi & Kramer 1996; Bonnet et al. 1999; Marras et al. 2003).

A fourth approach is the use of probes and primers whose antisense sequences have 1-2 mismatches to the intended target (Guo et al. 1997; Delihas et al. 1997). This approach can be successful if conventional allele-specific hybridization of a “perfect” antisense does not provide sufficient signal discrimination between its matching target and a closely related one, because the stabilities of the matched and mismatched duplexes are too similar. In such cases, introduction of sequence changes in the probes that create mismatches to both the intended and related targets can, if positioned correctly, increase the difference in stability between duplexes involving the intended vs. related targets. Interestingly, this method of increasing target specificity is often employed by natural antisense RNAs (Delihas et al. 1997; Kumar & Carmichael 1998; Zeiler & Simons 1998; Brantl 2002; Wagner et al. 2002).

Oligonucleotide probes that can be circularized after hybridization to single-stranded polynucleotide targets have great potential to be superior to linear hybridization probes. Because of the helical nature of nucleic acid duplexes, the circularized probes are wound around a single-stranded polynucleotide target, pseudo-topologically connecting the two polynucleotides through catenation, which provides increased stability of the probe-target complexes. It should be noted that true topological links can be formed only when circular probes are hybridized to circular targets or targets with cross-linked ends. These true topologically linked complexes can survive even under highly stringent washes that cause dissociation of ordinary duplexes. Also, circular nucleic acids may be amplified by RCA for detection and selection purposes (see below). Gryaznov & Lloyd (1995) pioneered the design of DNA Clamps, which can be circularized around the target using a chemical reaction between terminal non-nucleotide reactive groups. However, because of this unnatural internucleotide link, DNA clamps cannot be amplified by RCA.

Another type of circularizable probe was developed by Landegren and co-workers and called padlock probes (also known as C-probes or CLiPs) (Nilsson et al. 1994; Lizardi et al. 1998; Zhang et al. 1998; Kumar 1999; Thomas et al. 1999; Antson et al. 2000; Baner et al. 2001; Christian et al., 2001; Myer & Day 2001; Qi et al. 2001; Kuhn et al. 2002; Hardenbol et al. 2003). Padlock probes can detect point mutations and allow signal amplification by RCA. These probes are linear oligonucleotides designed so that their 15-20-nt terminal sequences, which are connected by a linker region, can hybridize to adjacent sites in the target DNA or RNA sequence. The terminal sequences can then be joined by DNA ligase. Because of the strict requirement of the ligase enzyme for perfect ends, the circularization efficacy of DNA padlocks is absolutely dependent on the purity of the material, which is challenging for such long (typically 70-100 nt) molecules (Kwiatkowski et al. 1996; Antson et al. 2000; Myer & Day 2001). The specificity and efficacy of DNA padlocks also relies on the fidelity and efficiency of DNA ligase for the ligation of substrate sequences on different templates. However, DNA ligases cannot perfectly discriminate single-nucleotide mismatched sequences (Wu & Wallace 1989; Luo et al. 1996; Pritchard & Southern 1997). In vitro selection experiments of sequences that can be ligated most efficiently by T4 DNA ligase (using substrate sequences with randomized nucleotides) showed that many of the selected sequences had one or more mismatches even at the ligation junction (Harada & Orgel 1993; James et al. 1998; Vlassov et al. 2004). Also, ligation of DNA termini aligned on RNA targets occurs with very low efficiency (Nilsson et al. 2000, 2001), thus limiting use of DNA padlocks for hybridization with DNA targets.

The probes of the present invention comprise antisense regions (regions that are complementary or substantially complementary to the target) and non-antisense regions (regions that are non-complementary to the target), which do not interact with the target. Multidomain polynucleotides comprising antisense and non-antisense regions have been previously reported. However, the probes of the present invention are distinct from these multidomain polynucleotides, as discussed below.

For example, antisense agents with hairpin structures at one or both ends of antisense agents have been reported (Noonberg & Hunt 1997). The hairpin structure(s) are used to increase the stability of the antisense agents against exonuclease degradation in cells. These are not for use as in vitro hybridization probes, and thus, were not demonstrated to improve the hybridization characteristics of these antisense molecules.

Similarly, oligonucleotides with a hairpin structure adjacent to one end of the antisense sequence of the hybridization probe or adjacent to a PCR primer probe have been reported (Walter et al. 1994; Lane et al. 1997, 1998; Ricceli et al. 2001; Chen et al. 2005). The hairpin end docks with the target's end through stacking interactions, thereby enhancing the stability of the short duplexes that have formed between antisense domain and the target sequence.

As another example, probes have been reported that contain stringency elements that are complementary to the antisense sequence and therefore serve to improve sequence-specificity by competing with the target for binding to the antisense sequence (Roberts & Crothers 1991; Hertel et al. 1998; Ohmichi & Kool 2000).

As another example, “molecular beacon”-like stem-and-loop probes have been reported which comprise a loop of antisense sequence that is complementary to a target sequence and a stem that is formed by the annealing of non-antisense arm sequences that flank the antisense sequence and that are complementary to one another (Tyagi & Kramer 1996; Bonnet et al. 1999). Such probes can exist in two conformations, linear and hairpin-shaped, and only the linear form can bind to the target. By providing a structure that competes with the target for binding to the antisense sequence, the “molecular beacon” is more sensitive to mismatches. However, the self-complementary arms are usually only 4-8 nt in length, since longer stems dramatically reduce hybridization rate and stability of probe-target duplexes.

In addition, hybridization probes and PCR primers have been reported that encode additional non-antisense sequence such as PCR primer sequence (usually universal, target-independent sequences) (Kataja et al. 2006), transcription promoters (e.g. T7, T3 and SP6) (Krupp 1988), Zip-code and Tag sequences (Mittman et al. 2007; Soderlund et al. 2008), and hairpin loop at the 5′-end which fluorescence significantly increased upon primer extension (Nazarenko et al. 1997). These non-antisense sequences have not been demonstrated to improve a hybridization characteristic of antisense and target sequence binding.

Surface-based hybridization. Both ordinary DNA arrays and sandwich-hybridization assays employ a reverse dot-blot format, in which target nucleic acids (DNA or RNA) are in solution while DNA probes are tethered to a surface (Ekins & Chu 1999; Tsai et al. 2003). Despite recent improvements, the arrays still suffer from limited specificity (ratio of target-specific to nonspecific, off-target hybridization), sensitivity (ratio of signal intensity to background noise, signal-to-noise ratio), and variability of results between different array platforms for low-copy genes (Tan et al. 2003; Marshall 2004; Kuo et al. 2006). Currently, DNA probes for surface-based hybridization are either synthesized in situ on a solid support or synthesized first and then spotted onto the array. Short, surface-bound oligonucleotides often have poor hybridization properties since hybridization on a solid surface is less efficient than solution hybridization (Peterson et al. 2002; Peplies et al. 2003). Tethering one end of an oligonucleotide probe to a surface reduces efficacy and specificity of hybridization to a target that is in solution. Also, nucleotide residues of the probe nearest the surface are less accessible to the target than those furthest away (Southern et al. 1999). Non-nucleic acid linkers, oligonucleotide spacers and simply longer oligonucleotides (≧60 nt) that move the probe sequence away from the surface are often used to enhance hybridization yields (Steel et al. 2000; Hughes et al. 2001).

In standard array experiments, mRNA targets are copied and amplified before application to the entire array chip and incubated for a long period of time (12-24 h) for effective hybridization. The hybridization and washing are usually done under conditions intended to be sufficiently denaturing to partially complementary duplexes but not target-specific hybrids. Selection of such conditions as well as design of target-specific probes is often compromised when target sequences with GC- or AT-rich clusters have to be assayed (Hacia, 1999). Such compromises are never perfect, often resulting in a substantial level of both false positives and false negatives. Therefore, results of array experiments must be validated by other methods that measure RNA levels, such as quantitative Northern blotting or qRT-PCR (Kothapalli et al. 2002).

While standard gene arrays can simultaneously access up to tens of thousands genes in a limited number of biological samples (currently ≦3), the reverse array format, in which DNA or RNA targets are surface-immobilized while oligo/polynucleotide probes present in hybridization solution, allows analysis of a few hundred genes in multiple biological samples. Dot blots, Northern blots, in situ hybridization, reverse expression microarrays (REM) and tissue microarrays share this same hybridization format (Player et al. 2004; Rogler et al. 2004). This format is very similar to solution hybridization since probe ends are untethered while the majority of target sequences are distant from the surface, and therefore can hybridize more efficiently and specifically than with the reverse dot-blot format.

Zip-code sequences. As mentioned above, one of the major challenges for multiplex detection of polynucleotides is the difficulty of optimizing the hybridization temperature for all of the capture probes and primers because of the wide variation in Tₘ for different sequences. To address the latter problem, several groups have developed sets of sequences that have similar melting temperatures and can be associated on a one-to-one basis with targets of interest. These sets include 24-nt “Zip-code” (Gerry et al. 1999), 25-nt “ZipCode” (Ye et al. 2001), and 20-nt (Fan et al. 2000) or 25-27-nt “Tag” (Hirschhorn et al. 2000) sequences. These sequence sets have been used in several microarray- and bead-based methods for mutation genotyping (Gerry et al. 1999; Fan et al. 2000; Favis et al., 2000; Hirschhorn et al. 2000; lannone et al. 2000; Ye et al. 2001; Song et al. 2005). Despite having different designs, the Zip-code and Tag sequences share several common features: (1) they are designed using computational approaches to be unique and to not hybridize either to one another or to sequences in the genome under study (e.g., the human genome); (2) they have similar thermodynamics and kinetics of hybridization so that hybridization and washing can be performed at a single stringency condition; and (3) individual sequences can be assigned (and re-assigned) to members of any set of target sequences simply by placing the two sequences in the same probe strand. The zip-code sequences have been previously used for the design of both hybridization probes and PCR primers (Shuldiner et al. 1990; Shuber et al., 1995; Kampke et al. 2001; Smith et al. 2001; Lin et al. 2006; Pinto et al. 2006).

Multiplex hybridization assays. Multiplex nucleic acid analysis by hybridization (MP), which allows the simultaneous, individual detection or measurement of multiple targets within the same sample, has several advantages over conventional singleplex experiments: (1) MP allows for significant savings in reagents, consumables and labor time since all samples are analyzed at once instead of in individual experiments; (2) MP reduces sample-to-sample variability because multiple measurements (including internal controls) are made in the same sample, whereas in traditional methods, a control test must be performed separately, under the inaccurate assumption that conditions were identical to the sample well (Ugozzoli 2004). Multiplexing technology, such as the Luminex xMAP platform, is particularly attractive for applications requiring a throughput of up to 1000 samples per day and multiplexing of from one to 100 tests per sample (Dunbar 2006). The xMAP technology uses microsphere beads (˜5 μm diameter) tagged with various proportions of two fluorescent dyes, providing up to 100 unique dye ratios that allow identification of individual beads by flow cytometry. Each bead set can be coated with a reagent specific to a particular bio-assay, allowing the capture and detection of specific analytes from a sample.

At the time of filing this application, QUANTIGENE® (Panomics, Inc) is the only commercially available method of multiplexed quantitative analysis of mRNAs in RNA extracts and cell lysates. This system combines sandwich hybridization, xMAP multi-analyte profiling beads (Luminex) and branched DNA (b-DNA) signal amplification technologies (Flagella et al. 2006). In QUANTIGENE® assays, target mRNAs from cell lysates or purified RNA extracts are captured to their respective (designated to specific targets) capture plates or microsphere beads using customized probe sets, which contain Capture Extender (CE), Label Extender (LE), and Blocker oligodeoxynucleotides that recognize a particular target during overnight hybridization at 53° C. followed by hybridization with branched DNA probe (bDNA) and 45 biotinylated label probes for 1 h at 46° C. After addition of streptavidin-conjugated Phycoerythrin (SAPE), the resulting fluorescence signal, which is proportional to the amount of mRNA captured on the bead surface, is analyzed on a LUMINEX® instrument. This and other methods, which rely on cooperative hybridization between the target mRNAs and multiple oligodeoxynucleotide probe sets (Collins et al. 1997; Tsai et al. 2003; Flagella et al. 2006), has several significant limitations.

First, the hybridization scheme is very complicated and requires rational design and optimization of more than a dozen different oligodeoxynucleotide hybridization probes for each RNA target. Second, the extensive secondary structures of RNA targets limit the number of sequences available for the hybridization of DNA sequences. Third, the reverse-dot-blot hybridization format used suffers from the same limitations inherent in DNA arrays including the need for lengthy, low-stringency hybridization and washing procedures since DNA-RNA duplexes are less stable than RNA-RNA complexes (see above). Both hybridization and washing conditions have to be optimized over the entire probe set for each target rather than for individual sense-antisense sequences, which can result in high background noise, low signal-to-noise ratio, and potentially increased levels of false-positives. Moreover, capturing the RNA targets through several different bead-linked oligonucleotide probes targeting different sites within the same target further complicates the optimization. Also, low concentrations of targets in cellular RNA extracts and cell lysates, which may be the result of either low natural abundance or as a result of knock-down experiments, can also result in lower efficacy of hybridization to probes as compared with more abundant transcripts since intermolecular hybridization between surface-bound probes is concentration-dependent, which can skew the quantification. Fourth, because this method relies on ordinary, lower-fidelity hybridization, it is not suitable for mutation analysis.

Multiplex PCR. Multiplex PCR would provide simultaneous amplification of many polynucleotide target sequences in one reaction under the same conditions, thus increasing the assay throughput and allowing more efficient use of each DNA sample. Most multiplex PCR reactions, however, are restricted to amplification of five to ten targets (Broude et al., 2001b; Broude et al., 2001c; Szemes et al. 2005). The reasons for this are that increasing number of primers required for specific amplification of different target sequences simultaneously increase a probability of primer-dimer formation and cross-hybridization of primers with non-target sequences. As a result, primer design for multiplex PCR is not a trivial task, requires tedious optimization of PCR conditions and often fails in experimental trials especially for short RNA targets with high variation of GC-contents such as miRNAs and other non-coding RNAs. Several approaches have been developed to overcome some of these difficulties.

One method, yielding rather uniform amplification of all PCR products, uses chimeric primers containing both target-specific and a universal or zip code sequences with two rounds of amplification. The first PCR round is performed with a relatively low concentration of such chimeric primers while the second round uses a high concentration of shorter primers complementary only to the zip code (Shuber et al. 1995). Special design of the universal primers forming “pan-handle” (hairpin) structures has also been proposed to suppress/decrease primer-primer interactions (Brownie et al. 1997). Additional improvement to the PCR multiplexing level (up to 30) is so-called the PCR suppression method, PS-PCR (Broude et al. 2001a; Broude et al. 2001b). DNA is first digested with a restriction enzyme and ligated with specially designed oligonucleotide adapters, which are about 40 nt long and have a high GC-content and are self-complementary. During the PCR denaturation-annealing steps, these adapters form strong double-stranded stems forcing each template DNA strand to form intrastrand stem-and-loop (hairpin) structures instead of perfect interstrand duplexes. Indeed, the intramolecular binding of self-complementary template ends is kinetically favored and more stable than the intermolecular binding to shorter so-called A-primers corresponding to the adapter sequences. Therefore, replication of such DNA templates using the universal (common for all targets) A-primers without target-specific T-primers, which are complementary to the target sequences located in single-stranded loop, is suppressed. However, such hairpin templates can be efficiently amplified in the presence of both the A- and T-primers. Because the A-primer is universal (the same for all targets), the PS-PCR allows amplification with only one target-specific primer thus reducing number of primers twofold compared with conventional PCR.

General problems with currently used hybridization probes. There are several problems with the hybridization probes that are currently used in the art. First, there is the trade-off between sensitivity and specificity. For example, long (>60 nt) polynucleotide probes provide enhanced efficacy of hybridization but lower sequence-specificity than short probes, whereas short (<25 nt) polynucleotide probes provide enhanced sequence-specificity but lower efficacy of hybridization than long probes. Second, hybridization is slow and ineffective: conditions that favor duplex formation also promote intrastrand structure formation in both probes and targets, but target fragmentation (performed to reduce intramolecular structure) reduces the signal and is applicable only to oligonucleotide arrays. Third, there is great difficulty in optimizing hybridization conditions for the simultaneous assay of both G:C- and A:T (or A:U)-rich targets. Fourth, when confronted with the limit of detection sensitivity, the skilled artisan must choose between obtaining or generating more target, e.g. through target amplification, or employing some method of signal amplification, for example by probe amplification, ELISA-based techniques, or sandwich hybridization with branched DNA/dendrimers.

Current developments in hybridization-based techniques have focused primarily on improvements of probe immobilization and data analysis rather than probe design. Thus, there is a need in the art for improvements in hybridization probe design.

### SUMMARY OF THE INVENTION

The present invention provides new hybridization probe designs and methods for their use in detection, identification, and quantitation of polynucleotide targets such as RNA and DNA.

Aspects of the invention include polynucleotide probes specific for a target polynucleotide which include: a) a target binding domain that is substantially complementary to a nucleotide sequence of a target polynucleotide; and b) a binding enhancer domain that cannot form a stable hybridization complex with a target polynucleotide or with a target binding domain under standard hybridization condition.

In some embodiments of the invention, the target binding domain ranges from 3-30 nucleotides in length. In some embodiments, the binding enhancer domain ranges from 30-10,000 in length. In certain embodiments, the binding enhancer domain forms a secondary or tertiary structure such as a stem-loop structure, a pseudoknot, a bipartite nucleic acid duplex, a multi-partite nucleic acid triplex or a multi-partite nucleic acid tetraplex. In certain embodiments, the secondary or tertiary structure is selected from sequences that are substantially related to a catalytically active hairpin ribozyme, a catalytically inactive hairpin ribozyme, a truncated hairpin ribozyme, a tRNA, or a region from a ribosomal RNA. In one embodiment, the minimal hairpin ribozyme catalyzes circularization of the polynucleotide probe. In certain embodiments, the binding enhancer domain comprises a first subdomain located 5′ to the target binding domain and a second subdomain located 3′ of the target domain. In certain embodiments, the polynucleotide probe ranges from 33-10,003 nucleotides in length.

The invention also provides for sets of polynucleotide probes, each set comprising at least two polynucleotide probes, each probe specific for a different target polynucleotide, In certain embodiments, each of the at least two polynucleotide probes comprises a unique identifier domain. In certain embodiments, the binding enhancer domain of each of the at least two polynucleotide probes is the same,

Aspects of the invention also include methods of identifying a target polynucleotide in a sample, including the steps of a) contacting a sample to a polynucleotide probe of the present invention under hybridizing conditions, wherein a target binding domain of the polynucleotide probe is substantially complementary to a nucleotide sequence of the target polynucleotide, and a binding enhancer domain of the polynucleotide probe provides for improved hybridization of the target binding domain to the target polynucleotide; and b) assaying for the presence of stable hybridization complexes between the polynucleotide probe and the target polynucleotide, so as to detect the presence of the target polynucleotide in the sample.

In certain embodiments, the binding enhancer domain forms a secondary or tertiary structure such as a stem-loop structure, a pseudoknot, a bi-partite nucleic acid duplex, a nucleic acid triplex or a nucleic acid tetraplex. In certain embodiments, this secondary or tertiary structure is encoded by sequence that is substantially related to a catalytically active hairpin ribozyme, a catalytically inactive hairpin ribozyme, a truncated hairpin ribozyme, a tRNA, or a region from a ribosomal RNA. In certain embodiments, the minimal hairpin ribozyme catalyzes circularization of the polynucleotide probe. In certain embodiments, the binding enhancer domain comprises a first subdomain located 5′ to the target binding domain and a second subdomain located 3′ of the target domain. In certain embodiments, the target polynucleotide or polynucleotide probe is captured or immobilized on a solid support. In certain particular embodiments, the solid support is a synthetic bead, a membrane or filter, a microarray slide, microtiter plate or microcapillary. In certain embodiments, the hybridization characteristic that is improved by the presence of the binding enhancing domain is selectivity, sensitivity, affinity or binding efficacy.

In certain embodiments of the method of the invention, the assaying step further includes a step to amplify the signal. In other embodiments, the assaying step further includes i) isolating hybridization complexes comprising the polynucleotide probe and said target polynucleotide; ii) recovering the polynucleotide probe from the hybridization complexes; iii) hybridizing a synthesis primer to the recovered polynucleotide probe; iv) placing the synthesis primer-hybridized polynucleotide probe under nucleic acid synthesis conditions to extend the synthesis primer; and v) detecting the extended synthesis primer.

In certain embodiments of the method of the invention, multiple target polynucleotides are detected in the sample using multiple polynucleotide probes, each specific for one of the multiple target polynucleotides. In some embodiment, each of the multiple polynucleotide probes in addition to target-specific antisense sequence comprises a unique identifier domain, which could be a Zip-code sequence or insert of a defined number of nucleotides. In some embodiments, the target polynucleotides are captured on a solid support, and the assaying step includes: i) isolating hybridization complexes comprising the multiple polynucleotide probes and their corresponding target polynucleotides; ii) recovering the polynucleotide probes from the hybridization complexes; iii) amplifying and labeling the recovered polynucleotide probes; iv) hybridizing the labeled polynucleotide probes to multiple second polynucleotide probes arrayed on a solid support, each second polynucleotide probe comprising a sequence from one of the target sequences; and v) detecting the labeled probes.

### BRIEF DESCRIPTION OF THE FIGURES

FIG. 1. Lasso structure and self-processing properties. A: Consensus structure of the hairpin ribozyme (HPR). The HPR is derived from sequences in the minus strand of Tobacco ringspot virus satellite RNA. The site-specific RNA cleavage induced by the ribozyme generates fragments having 2′,3′-cyclic phosphate and 5′-OH termini. HPR can efficiently ligate those ends and can exist as linear and circular forms that interconvert. The internal equilibrium between circular and linear forms depends on the relative stability of the cleaved and ligated forms and ionic conditions. Dots represent any nucleotide (A, U, G or C), dashes represent required pairings, V is ‘not U’ (A, C, or G), Y is a pyrimidine (U or C), R is a purine (A or G), B is ‘not A’ (U, C or G), H is ‘not G’ (A, C or U) (Berzal-Herranz & Burke, 1997). B. Scheme of Lasso self-processing. Self-trimming of original unprocessed transcript (UP) generates half-processed intermediates (5′-HP and 3′-HP, correspondingly) and fully processed unligated, linear form (L) that can convert into the circular form (C) through self-ligation.

FIG. 2. Lasso I design. Lasso I can be transcribed as a precursor (A) that undergoes self-processing. The mature Lasso can equilibrate between linear (cleaved) and circular (ligated) forms (B). Lasso I, containing a target-specific antisense sequence (shown in red; poly “N” sequence) can sequence-specifically bind to and circularize around a polynucleotide target (rectangular box hybridizing to poly “N” sequence) forming pseudo-topological link (C), which is stronger than an ordinary duplex. True topological linkage can be formed if the polynucleotide target is either circular or if slippage of the circular Lasso from the linear target is restricted (e.g., target is cross-linked to a surface).

FIG. 3. Lasso II design. A: The Lasso, whose multifunctional design combines hairpin ribozyme (HPR) and antisense moieties, can be transcribed to form a precursor that then undergoes self-processing (cleavage and ligation) at the sites shown. B: The mature Lasso II can equilibrate between linear (cleaved) and circular (ligated) forms. In the absence of target, the Lasso occurs predominantly in the circular form. C: Lasso II cannot circularize around a polynucleotide target after hybridization because the ligation half-substrate sequences (shown in green and blue) are pulled apart by the formation of the duplex. Instead, the ability of the circular form (which has a “closed” conformation like a “molecular beacon”) to interconvert into the linear form can enhance the sequence specificity with which it can form an open complex with a target. Lasso circularization also allows RCA signal amplification, providing a straight forward method for optimization of antisense sequence through iterative selection and amplification (see EXAMPLES below).

FIG. 4. Binding of Lasso ATR1 to TNFα RNA in solution. A: Putative structure of the complex between TNF-709 (MuTNF mRNA), an RNA consisting of nt 280-988 of murine TNFα mRNA, and the fully processed ATR1 Lasso (which targets nt 562-583 of the murine TNF message). B: Kinetics of binding of ATR1 with TNF RNA. A ³²P-labeled TNF target was incubated with cold ATR1 Lasso at 37° C. for the time periods indicated above each lane. Incubation was carried out in 10 mM MgCl₂, 50 mM Tris-HCl (pH 8.0) either with (left panel) or without 20% formamide (right panel). Following complex formation, reactions were quenched with one volume of FLS loading buffer (95% formamide, 12 mM EDTA). C: Heat-induced dissociation of complexes formed by TNF target RNA with linear and circular Lasso species. ³²P-labeled Lassos were incubated at 37° C. for 2 hrs in buffer containing 10 mM MgCl₂, 50 mM Tris-HCl (pH 8.0), with or without non-radioactive TNF-705 (in excess over Lassos) as indicated. Reactions were quenched by addition of an equal volume of the FLS loading buffer. Samples in lanes 3-6 were additionally incubated for 2 min at 50°, 65°, 80°, and 95° C., respectively and transferred immediately to ice to prevent re-hybridization. Products were analyzed by 6% denaturing PAGE (8M Urea).

FIG. 5. Analysis of the interaction of circular and linear targets with linear and circular Lassos in solution. A: Schematic of Lasso 229-7.0 bound to linear target RNA (MuTNF mRNA). B: Schematic of Lasso 229-7.0 bound to circular target RNA. C: Internally ³²P-labeled Lasso 229-7.0 was incubated with linear or circular target RNA (as indicated) in buffer containing 50 mM Tris-HCl (pH 7.5), 20% formamide and either 10 mM MgCl₂ (lanes 1-6) or 10 mM EDTA (lanes 7-12) for 120 min at 37° C. The reactions were quenched as described in the legend to FIG. 3C and each sample was divided into two halves. One half was loaded onto a denaturing 6% polyacrylamide gel containing 8M urea without further incubation (lanes 2, 5, 8, 11), and the other half of each sample was incubated at 95° C. for 5 min and then placed on ice (lanes 3, 6, 9, 12) before loading onto the same gel. As controls, Lassos were incubated in the same buffers without target (lanes 1, 4, 7, and 10).

FIG. 6. Selection scheme for Lasso species that efficiently bind to and circularize around target RNA. A: Sequence and secondary structure of unprocessed Lasso containing a gene-specific (directed) library, showing position of RT primer 1 used in Panel B (arrow). B: Scheme to selectively amplify and transcribe those Lasso species that are capable of circularization around a target. RT primer 1 selectively extends only circular Lassos, yielding single-stranded DNA multimers of the Lasso sequence. Two additional primers (T7 PCR primer 2 and PCR primer 3—see Panel C) amplify the RT product by PCR and restore the T7 promoter sequence at the 5′-end of the Lasso to allow transcription by T7 RNA polymerase. C: Structure of a self-processed circular Lasso bound to its complementary site in TNFα mRNA, showing the position of the primers designed to selectively amplify circular Lassos from selection scheme of Panel D. D: Multiple-round scheme for selecting Lassos capable of binding to and circularizing around the TNF target RNA using a gel-shift assay and the amplification procedures shown in Panels A-C.

FIG. 7. Lassos TNF4 and TNF4-DB can discriminate between perfectly matched and mismatched 1000 nt-long target RNAs. A: Sequence and putative structure of Lasso TNF4. B: Sequence and presumed structure of unprocessed Lasso TNF4-DB. The TNF4-DB sequence is the same as that of TNF4 except for the added stringency element (hybridized sequence on the right; yellow highlight), which is complementary to the Lasso antisense segment (pink). Positions of nucleotides opposite the mutations (see Panel C) in the target RNA are shown in blue (indicated with an x in panel A). C: Mutations introduced by site directed mutagenesis in murine TNFα mRNA to create mismatches with antisense segments of Lassos TNF4 and TNF4-DB are shown in red (arrows). D: Binding of TNF4 with perfectly matched (wt) and mismatched target RNAs containing 1-3 mismatches (4-1, 4-21, 4-22, 4-3). Internally ³²P-labeled Lassos were incubated either alone (−) or with non-radioactive target RNA at 37° C. for 1 h in SB buffer. Reactions were quenched with formamide buffer containing 10 mM EDTA. Products were analyzed by denaturing 5% PAGE (8M urea). E: Lasso TNF4-DB binding assay with perfectly matched (wt) and mismatched target RNAs containing 1-3 mismatches (4-1, 4-21, 4-22, 4-3). Incubation and electrophoresis were carried out as for Panel D.

FIG. 8. Dot-blot assay of hybridization of Lasso probe to a target RNA in a background of total cellular RNA. Excess ³²P-labeled Lasso TNF4 was hybridized overnight at 37° C. to unlabeled TNF1000 target RNA mixed with total cellular RNA (as indicated by each dot) that was previously immobilized by UV-cross-linking to a nylon membrane (see text for details). Each panel is a phosphorimage of the membrane washed at the indicated temperature at low ionic strength (0.1×SSC/0.1% SDS /1 mM EDTA). Signal-to-noise ratios were quantitated and listed to the right of each spot. In the mock-treated spot, only buffer without any RNA was spotted on the membrane. It can be seen that washing under more stringent conditions (here, higher temperature) increases the signal-to-noise ratio.

FIG. 9. Scheme 1 for multiplex detection of immobilized polynucleotide targets using Lasso probes. Here, target polynucleotides are first extracted from biological samples and then immobilized on a filter or membrane. FIG. 9A describes the capture of target-specific probes on immobilized targets and recovery of the captured probes. The recovered probes are then hybridized with oligonucleotide primers attached at their 5′ ends to either color-coded beads (FIG. 9B-C) or slide arrays (FIG. 9D) and extended by a reverse transcriptase or DNA polymerase. The extension products can be detected either by sandwich hybridization assays using either standard ELISA or bDNA/DNA dendrimer techniques (FIGS. 9B and 9D, panel a) or rolling circle amplification, RCA (FIGS. 9C and 9D, panel b).

FIG. 10. Scheme 2 (alternative) for multiplex detection of polynucleotide targets using Lasso probes. Here, the RNA targets are captured onto the surface of magnetic beads directly from cell/tissue lysates. Target capture can be done either using chemical/UV cross-linking or by hybridization with target-specific oligonucleotides attached to the magnetic beads at their 5′ ends. Such oligonucleotides should provide (either through chemical modification, or extended length, or primer extension) high affinity to the target to survive stringent hybridization with Lasso or other amplifiable polynucleotide probes. The probes, which become captured on magnetic beads through hybridization with the immobilized targets, are recovered and enzymatically amplified (e.g., by RT-PCR and/or transcription). The amplified probes are labeled either during transcription or by direct chemical modification. The labeled probes are then detected by hybridization with second polynucleotide probes comprising synthetic monomers or multimers of the target sequences which were arrayed on a solid support (such as color-coded beads, or 2D slide arrays, or 3D gel arrays, or dot/slot blots, or microcapillaries). Alternatively, the amplified and labeled target-specific probes can be detected by mobility shift assays by gel or capillary electrophoresis.

FIG. 11. Sequence and secondary structure of RNA Lasso probes HCV3 (top) and TNF4 (bottom) that target HCV RNA and TNF mRNA, respectively. The Lasso antisense sequences are shown in red and underlined.

FIG. 12. Comparative hybridization of polynucleotide probes (RNA Lasso HCV3, DNA oligo-61 and oligo-23) with immobilized HCV RNA targets demonstrating that Lasso probes detect RNA targets with greater sensitivity than ordinary short (23 nt) or long DNA (61 nt) probes. All probes share the same 23-nt long antisense sequence besides oligo-61, which has additional 38 nt complementary to the RNA target. The ³²P-labeled probes were hybridized either for 1 hr at 37° C. (A) or overnight (B) in Church buffer and washed in 0.1×SSC/0.1% SDS/1 mM EDTA at 50° C. (oligo-23) and 65° C. (HCV3 Lasso and oligo-61) (see Example 5 for more details). Dot-blot hybridizations assays were performed in triplicate. Plots of signal-to-noise ratio vs. amount of HCV target UV-crosslinked to the membrane are shown.

FIG. 13. Structure for RNA Lasso probes TNF4 (top) and TNF4-MB9 (bottom). Lasso antisense sequence complementary to TNFα mRNA are underlined (TNF4) or marked by an arc (TNF4-MB9). Arrows show site for (optional) cleavage and ligation by hairpin ribozyme domain.

FIG. 14. Comparison of hybridization of polynucleotide probes (RNA Lasso TNF4-MB9, 21 nt-DNA and 61 nt-DNA) to the immobilized TNFα RNAs indicating enhanced discrimination of mismatches in RNA targets by Lasso probe. Lasso and 21 nt-DNA probes share the same 21-nt long antisense sequence while 61-probe has this antisense sequence extended to 61 nt complementary to the target. A: The dot-blot hybridization of the ³²P-labeled probes with RNA targets containing different amounts of mismatches to the probe antisense sequences. The RNA targets were UV-crosslinked to membrane at 5 and 50 ng amount per spot, hybridized overnight at 37° C. and unbound probes were washed in 0.1×SSC/0.1% SDS /1 mM EDTA at the indicated temperatures. The detected signal-to-noise (S/N) ratios are shown along with hybridization spots. Note: the 21-nt DNA probe is completely washed away at the 65° C. condition. B: 21-nt antisense sequence (AS) of the probes is shown along with the fully complementary TNF RNA target sequences as well as the target mutants containing one (4-1), two (4-21 and 4-22) and tree (4-3) mismatches. Note: the TNF targets were the same as shown in FIG. 7C.

FIG. 15. Comparative hybridization of polynucleotide probes (RNA Lasso TNF4, 21 nt-DNA and 61 nt-DNA) to the immobilized TNFα DNAs, indicating enhanced discrimination of mismatches in DNA targets by Lasso probe. Lasso and 21 nt-DNA probes share the same 21-nt long antisense sequence while 61-probe has this antisense sequence extended to 61 nt complementary to the target. The probe antisense sequences as well as DNA target sequences containing different amounts of mismatches to the probe antisense sequences were identical to those shown in FIGS. 7C and 14B for RNA targets except T→U substitutions in the DNA. The double-stranded DNA targets were denatured and immobilized on nylon membrane by UV cross-linking. Dot-blot hybridizations of ³²P-labeled probes with the immobilized DNA targets were performed in triplicates overnight at 37° C. and unbound probes were washed in 0.1×SSC/0.1% SDS/1 mM EDTA at 50° C.

FIG. 16. Comparative hybridization of polynucleotide probes (RNA Lasso TNF4, TNF4-MB9, 21 nt RNA and 61 nt DNA oligonucleotides) to the immobilized TNFα RNAs indicating enhanced sensitivity of Lasso probes in detection of RNA targets in comparison to ordinary short RNA and long DNA probes. A: The dot-blot hybridization of the ³²P-labeled probes with TNFα mRNA target. The different, indicated amounts of TNF RNA were UV-crosslinked to membrane, hybridized overnight at 37° C. and unbound probes were washed in 0.1×SSC/0.1% SDS/1 mM EDTA at 65° C. (all experiments done in triplicate). All probes contain the same 21 nt antisense sequence with the exception of the 61 nt DNA probe which forms an extra 40 bp with the RNA target. The detected signal-to-noise (S/N) ratios are shown along with hybridization spots. B: Plots for signal-to-noise (S/N) ratios calculated from the experiments shown in panel A. Note: Lasso Structural variations in non-antisense sequences of Lasso TNF4 and TNF4-MB9 probes, can affect binding efficiency of the same antisense sequence.

FIG. 17. Comparative hybridization of polynucleotide probes (RNA Lasso TNF4, TNF4-MB9, RNA21 and DNA21) to the immobilized TNFα RNAs indicating enhanced both sensitivity and sequence-specificity of Lasso probes in detection of RNA targets in comparison to ordinary short RNA and DNA probes. All probes contain the same 21 nt antisense sequences. RNA targets represent a model TNF RNA wild type (wt) and its site-specifically mutated versions. The probe antisense sequences as well as DNA target sequences containing different amounts of mismatches to the probe antisense sequences were identical to those shown in FIGS. 7C and 14B. A: Signal-to-noise (S/N) ratios determined in for dot-blot experiments using ³²P-labeled probes that were hybridized at 37° C. with the different amount of UV-crosslinked, non-radioactive TNF RNA targets. The unbound probes were then washed with 0.1×SSC, 0.1% SDS, 1 mM EDTA at 65° C. with exception of 21 nt DNA probe, which was washed at 50° C. since it completely dissociated at the higher temperature. B: Reduction in S/N values for the mutated targets relative to the wt for the tested probes. Note: An additional structural elements in the Lasso probes provide both higher sensitivity and sequence specificity than those for short RNA and DNA probes.

FIG. 18. A: Sequences of 7 common genotypes of the HCV genomic RNA in the conserved IRES element of nt 195-224. Alignment is shown relative to genotype 1b, the common genotype found in infected patients in the US and Western Europe. B: Structure of genotype-specific HCV Lasso probes. Indicated antisense sequences are complementary to the corresponding target sequences shown in panel A. Nucleotides that differ from genotype 1b are shown in red and underlined in both sense (target) and antisense (probe) sequences shown in 5′-3′ direction.

FIG. 19. Genotype-specific dot blot hybridization of Lasso probes to immobilized target RNAs corresponding to main HCV IRES genotypes. Each Lasso probe was also tested for cross-hybridizations with non-matching genotypes to check potential false-positives and false-negative results. Genotype-specific RNA targets were spotted in discrete dots and UV-crosslinked to membrane and then hybridized with ³²P-labeled Lasso probes in Church buffer overnight at 37° C. Unbound probes were washed in 0.1×SSC/0.1% SDS/1 mM EDTA at 60° C. Sequences of target sites and antisense sequences of the Lasso probes are shown in FIG. 18.

FIG. 20. Structure of polynucleotide probes NP1 (left) and NP2 (right) derived from Lasso TNF4. Antisense sequences complementary to TNF RNA target are the same in both probes and are marked by arc. Arrow show site for (optional) cleavage and ligation in NP1 by hairpin ribozyme domain. Note: the sequence of HPR domain in NP2 is reversed (3′-5′) in comparison to those in NP1 and Lasso TNF4.

FIG. 21. Sensitivity (signal-to-noise ratio, S/N) of polynucleotide probes (Lasso TNF4, Lasso TNF4-MB9, NP1 and NP2) in detection of immobilized TNF RNA target in the presence of total cellular RNA. Antisense sequences complementary to TNF RNA target are the same in all probes. Signal-to-noise (S/N) ratios were calculated for dot-blot experiments done in triplicate. Different amounts (as indicated) of non-radioactive TNF RNA targets pre-mixed with total cellular RNA (250 ng per spot) were spotted to membrane and UV-crosslinked before overnight hybridization with ³²P-labeled probes at 37° C. The unbound probes were then washed with 0.1×SSC, 0.1% SDS, 1 mM EDTA at 65° C.

FIG. 22. Sensitivity (signal-to-noise ratio, S/N) of polynucleotide probes (Lasso TNF4, Lasso TNF4-MB9, NP1 and NP2) in detection of immobilized TNF RNA target in the absence of total cellular RNA. Antisense sequences complementary to TNF RNA target are the same in all probes. Signal-to-noise (S/N) ratios were calculated for results of dot-blot experiments done in triplicate. Different amounts (as indicated) of non-radioactive TNF RNA targets were spotted to membrane and UV-crosslinked before overnight hybridization with ³²P-labeled probes at 37° C. The unbound probes were then washed with 0.1×SSC, 0.1% SDS, 1 mM EDTA at 65° C. Note: Comparison of results shown in FIGS. 22-23 suggests that the presence of total RNA did not significantly reduce sensitivity of detection of target RNA.

FIG. 23. Structure of polynucleotide probes NP3 (left) derived from Lasso TNF4, and NP4 (right) derived from Lasso TNF4-MB9. Antisense sequences complementary to TNF RNA target are the same in both probes and are marked by arc.

FIG. 24. Sensitivity of polynucleotide probes (RNA: Lasso TNF4-MB9, NP1, NP2, NP3 and NP4: DNA: oligo-21 and oligo-61) in detection of immobilized TNF RNA target. All probes share the same 21-nt long antisense sequence besides oligo-61, which has additional 40 nt complementary to the RNA target. Signal-to-noise (S/N) ratios were calculated for results of dot-blot experiments done in triplicate. Different amounts (as indicated) of non-radioactive TNF RNA targets were spotted to membrane and UV-crosslinked before overnight hybridization with ³²P-labeled probes at 37° C. The unbound probes were then washed with 0.1×SSC, 0.1% SDS, 1 mM EDTA at 65° C. with exception of 21 nt DNA probe, which was washed at 50° C. since it completely dissociated at the higher temperature.

FIG. 25. Efficiency of mismatches discrimination in immobilized homologous RNAs by RNA (Lasso TNF4-MB9, NP1, NP2, NP3 and NP4) DNA (oligo-21 and oligo-61) polynucleotide probes. All probes share the same 21-nt long antisense sequence besides oligo-61, which has additional 40 nt complementary to the TNF RNA targets. The 21-nt antisense sequence is fully complementary to the wild type TNF RNA while has mismatches to the mutated RNA targets: one (4-1), two (4-21 and 4-22) and tree (4-3), respectively. The TNF RNA targets were the same as shown in FIGS. 7C and 14B. A: A bar graph of signal-to-noise (S/N) ratios for each RNA target and each polynucleotide probe. Signal-to-noise (S/N) ratios were calculated for dot-blot experiments done in triplicate. Non-radioactive TNF RNA targets were spotted to membrane and UV-crosslinked before overnight hybridization with ³²P-labeled probes at 37° C. The unbound probes were then washed with 0.1×SSC, 0.1% SDS, 1 mM EDTA at 65° C. with exception of 21 nt DNA probe, which was washed at 50° C. since it completely dissociated at the higher temperature. B: Efficiency in the mismatch discrimination calculated as N-fold reduction in S/N values for the mutated targets (indicated in right vertical lane) relative to the wt for the tested probes (indicated in top horizontal lane).

FIG. 26. Structure of polynucleotide probes PCR2 (top) and PCR3 (bottom) derived from Lasso TNF4-MB9. Antisense sequences complementary to TNF RNA target are the same in both probes and are marked by arc. Arrow show site for (optional) cleavage and ligation in NP1 by hairpin ribozyme domain. Note: Hairpin ribozyme stem A present in PCR2 probe has been deleted in PCR3 construct.

FIG. 27. Sensitivity of related RNA probes (Lasso TNF4-MB9, Lasso TNF4, PCR2, PCR3, NP1, NP2, NP3 and NP4) in detection of immobilized TNF RNA target in the absence of total cellular RNA. All probes share the same 21-nt long antisense sequence complementary to the RNA target. Signal-to-noise (S/N) ratios were calculated for results of dot-blot experiments done in triplicate. Different amounts (as indicated) of non-radioactive TNF RNA targets were spotted to membrane and UV-crosslinked before overnight hybridization with ³²P-labeled probes at 37° C. The unbound probes were then washed with 0.1×SSC, 0.1% SDS, 1 mM EDTA at 65° C. Note: Relative sensitivity of the probe tested is as follows: MB9≧PCR3>PCR2>NP2>TNF4>NP1>NP3≈NP4.

FIG. 28. Sensitivity of related RNA probes (Lasso TNF4-MB9, Lasso TNF4, PCR2, PCR3, NP1, NP2, NP3 and NP4) in detection of immobilized TNF RNA target in the presence of total cellular RNA. All probes share the same 21-nt long antisense sequence complementary to the RNA target. Signal-to-noise (S/N) ratios were calculated for results of dot-blot experiments done in triplicate. Different amounts (as indicated) of non-radioactive TNF RNA targets were pre-mixed with total cellular RNA (250 ng per spot), spotted to membrane and UV-crosslinked before overnight hybridization with ³²P-labeled probes at 37° C. The unbound probes were then washed with 0.1×SSC, 0.1% SDS, 1 mM EDTA at 65° C. Note: Comparison of results shown in FIGS. 27-28 suggests that presence of total RNA can slightly change the relative target-specific sensitivity of the probe tested.

FIG. 29. Structure of RNA polynucleotide probes NP5 (top) and NP6 (bottom) derived from Lasso TNF4-MB9. Antisense sequences complementary to TNF RNA target are the same in both probes and are marked by arc. Note: Hairpin ribozyme stem B present in TNF4-MB9 has been deleted in these constructs. NP5 can form stem duplex flanking antisense sequence whereas NP6 cannot.

FIG. 30. Structure of RNA polynucleotide probes NP7 featuring extended double stem-loop (DSL) at both ends of antisense sequences. The underlined 21-nt antisense sequence complementary to TNF RNA target is the same as in Lassos TNF4-MB9/TNF4 and their derivatives, however other sequences are unrelated.

FIG. 31. Sensitivity of non-Lasso NP7 probe compared to Lasso-related RNA polynucleotide probes (Lasso TNF4-MB9, NP1, NP2, NP5 and NP6) in detection of immobilized TNF RNA target in the presence of total cellular RNA. All probes share the same 21-nt long antisense sequence complementary to the RNA target. Signal-to-noise (S/N) ratios were calculated for results of dot-blot experiments done in triplicate. Different amounts (as indicated) of non-radioactive TNF RNA targets were pre-mixed with total cellular RNA (250 ng per spot), spotted to membrane and UV-crosslinked before overnight hybridization with ³²P-labeled probes at 37° C. The unbound probes were then washed with 0.1×SSC, 0.1% SDS, 1 mM EDTA at 65° C. Note: Relative sensitivity of the probe tested is as follows: TNF4-MB9>NP5≈NP7≈NP2>NP6≈NP1>TNF4.

FIG. 32. Sensitivity of non-Lasso NP7 probe compared to Lasso-related RNA polynucleotide probes (Lasso TNF4-MB9, NP1, NP2, NP5 and NP6) in detection of immobilized TNF RNA target in the absence of total cellular RNA. All probes share the same 21-nt long antisense sequence complementary to the RNA target. Signal-to-noise (S/N) ratios were calculated for results of dot-blot experiments done in triplicate. Different amounts (as indicated) of non-radioactive TNF RNA targets were spotted to membrane and UV-crosslinked before overnight hybridization with ³²P-labeled probes at 37° C. The unbound probes were then washed with 0.1×SSC, 0.1% SDS, 1 mM EDTA at 65° C. Note: Relative sensitivity of the probe tested is as follows: NP5>TNF4-MB9≈NP7>NP2≈NP6>TNF4>NP1. Comparison of results shown in FIGS. 31-32 suggests that presence of total RNA can slightly change the relative target-specific sensitivity of the probe tested.

FIG. 33. Efficiency of single mismatch (or single-nucleotide polymorphism, SNP) discrimination in homologous RNAs by RNA polynucleotide probes: Lasso TNF4-MB9, TNF4, NP1, NP2, NP5, NP6 and NP7. All probes share the same 21-nt long antisense sequence besides oligo-61, which has additional 40 nt complementary to the TNF RNA targets. The 21-nt antisense sequence is fully complementary to the wild type TNF RNA while has one mismatch to the 4-1 mutated RNA targets. The TNF RNA targets were the same as shown in FIGS. 7C and 14B. A: A bar graph of signal-to-noise (S/N) ratios for each RNA target and each polynucleotide probe. Signal-to-noise (S/N) ratios were calculated for dot-blot experiments done in triplicate. Non-radioactive TNF RNA targets were spotted to membrane and UV-crosslinked before overnight hybridization with ³²P-labeled probes at 37° C. B: Efficiency in the mismatch discrimination calculated as N-fold reduction in S/N values for the mutated target relative to the wt for the tested probes (indicated in top horizontal lane).

FIG. 34. Schematic representation of different structures for polynucleotide probes that have improved/enhanced hybridization characteristic as compared to ordinary hybridization probes. Such polynucleotide probes can match or exceed the sequence specificity of short hybridization probes and the binding efficiency of long probes. These superior hybridization probes comprise two or more functional domains including: (i) a short (3-30 nt) target-binding domain that is substantially complementary to a target polynucleotide (shown by solid line); (ii) a binding enhancer domain of 30 nt or more that cannot form a stable complex with either the target polynucleotide or the target binding domain under stringent hybridization conditions; and optionally other domains that may be required for probe detection or amplification or both. The binding enhancer domain could be either unfolded or folded in various secondary (A-H) or tertiary (I) structures. The presence of folded structures is preferred since they can reduce background signal through minimization of non-specific, accidental binding of the polynucleotide probes to non-intended targets. The simplest examples of secondary structures are duplexes (D-E, G-H) and hairpins (A-C, H-I). The simplest examples of tertiary structures are complexes between hairpin loops (I), and complexes between duplexes with internal loops and bulges (as, for example, with Loop A and Loop B in the hairpin ribozyme; see FIG. 1). Other examples of well-known tertiary structures include pseudoknots, triplexes and tetraplexes. The binding enhancer domains can be located at 5′ (A and D) or 3′ (B and E) ends, or at both ends (C, F-I) of the target binding domain. Also, the binding enhancer domain can be formed by a single-strand (folded or unfolded) (A-C, G-I) or by two substantially complementary separate strands (D-F). Moreover, the binding enhancer domains that are located at both ends of the target-binding domain could interact with each other (G-I) as in the Lasso probes described here.

### DETAILED DESCRIPTION OF THE INVENTION

Before the hybridization probes and methods of use according to the present invention are described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

All patents and other references cited in this application are incorporated into this application by reference except insofar as they may conflict with those of the present application (in which case the present application prevails). The citation of any publication is for its disclosure prior to the filing date and should not be construed as an admission that the present invention is not entitled to antedate such publication by virtue of prior invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

“Complementary” and its equivalents as used herein are meant to describe the ability of a polynucleotide sequence to form perfect, continuous classic (Watson-Crick, WC) base-pairing with another polynucleotide sequence, as, for example, in the ability of an antisense sequence to perfectly base-pair with a target sequence. Complementary nucleotides are, generally, A and T (or A and U), or C and G. By “substantially complementary” is meant that a few wobble/non-classic base-pairs or mismatches may be present without sacrificing the sequence specificity of target binding. Two single stranded RNA or DNA molecules are said to be substantially complementary when the nucleotides of one strand, optimally aligned with appropriate nucleotide insertions or deletions, pair with at least 80% of the nucleotides of the other strand, including at least 90% to 95%, as well as from 95 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, including at least about 75%, at least 90% complementary or more. See, M. Kanehisa Nucleic Acids Res. 12:203 (1984), incorporated herein by reference.

A “Duplex” is said to exist if at least two oligonucleotides and/or polynucleotides that are fully or partially complementary undergo Watson-Crick-type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base-pairing with a nucleotide in the other strand. The term “duplex” comprehends the pairing of nucleoside analogs, such as deoxyinosine, nucleosides with 2-aminopurine bases, PNAs, LNA's and the like, that may be employed. A “mismatch” in a duplex between two oligonucleotides or polynucleotides means that a pair of nucleotides in the duplex fails to undergo Watson-Crick bonding.

“Hybridization” as used herein is used to refer to the technique of allowing polynucleotide sequences with some amount of complementarity to bind to one another. Hybridization usually involves the steps of 1) allowing binding between probe and target; and 2) washing away unbound or weakly bound probes under stringent conditions, wherein stringent hybridization conditions are those washing conditions that provide dissociation for imperfect complexes while preserving the intended complexes between target-specific probes and corresponding targets. Improvements in hybridization characteristics can be improvements in the selectivity of hybridization (sequence specificity and mismatch discrimination), the sensitivity of hybridization (ratio of absolute signal to background signal, signal-to-noise ratio), the affinity between probe and target (ratio of binding rate to dissociation rate between hybridization probes and targets); the stability of the duplex or complex (thermal stability, Tm; also kinetic inertness of dissociation or kinetic trap), or the efficiency or efficacy of hybridization (hybridization rate and/or yield of complex between probe and target for a fixed time of incubation under hybridization conditions).

By “unique identifier domain” is meant a domain on a polynucleotide probe that is unique to that probe as compared to any other polynucleotide probe employed in a multiplex assay (an assay using multiple polynucleotide probes simultaneously). Unique identifier domains can thus be interrogated to positively identify each polynucleotide probe present in a sample (e.g., after isolation of hybridization complexes between multiple polynucleotide probes and theri corresponding targets). In addition to a target-specific antisense sequence, which is an “identifier” itself, a probe can have an additional identifier domain, which could be a Zip-code sequence or an insert of a defined number of nucleotides. Unique identifier domains can range from 4 to 400 nucleotides in length.

### Hybridization Probes

Ordinary short oligonucleotide probes usually provide higher sequence-specificity but lower efficacy of hybridization than longer ordinary polynucleotide probes where both are fully complementary to the target polynucleotide. The polynucleotide probes according to the present invention combine the hybridization efficacy of long probes with the sequence-specificity of short probes. Moreover, these probes provide higher affinities toward their polynucleotide targets than short hybridization probes as well as increased sensitivity (signal-to-noise ratio) for target detection in comparison to ordinary long hybridization probes, which themselves are more sensitive than the shorter probes.

In certain embodiments, polynucleotide probes according to the present invention range from 30 to 10,000 nucleotides (nt) in length, for example, from 50 to 2000 nt in length, including from 100-500 nt. In certain embodiments, a polynucleotide probe according to the invention contains a single polynucleotide sequence, while in other embodiments, a polynucleotide probe contains multiple polynucleotide sequences (so called bi-partite, tri-partite, tetra-partite, etc., as discussed in further detail below). Polynucleotide probes according to aspects of the present invention can include virtually any kind of nucleotide base, including (but not limited to) unmodified RNA bases, unmodified DNA bases or both (e.g., RNA-DNA chimeric polynucleotides) as well as one or more chemically modified RNA or DNA residue. Depending on their length and the presence of modified residues, the probes can be either chemically synthesized or prepared by enzymatic polymerization using techniques known in the art. In addition to ordinary RNA and DNA polymerases, mutated or engineered versions of polymerase enzymes can be used to incorporate into the probes variety of modified nucleotides (see, e.g., BACKGROUND AND RELATED ART section above).

In certain embodiments, the polynucleotide probes can be modified to include additional functional moieties (also called modified polynucleotide probes). Exemplary functional moieties include, without limitation, radioactive and fluorescent labels as well as anchor ligands such as biotin or digoxigenin. The functional moieties can be located internally or at either end of the probes. Probe modification can be carried out post-synthetically by chemical or enzymatic reactions such as ligation or polymerase-assisted extension. Alternatively, internal labels and anchor ligands can be incorporated into probes directly during enzymatic polymerization reactions using trace amounts of modified NTPs as substrates.

In certain embodiments, polynucleotide probes of the present invention include a target binding domain and a binding enhancer domain. In certain embodiments, the “target binding domain” ranges in length from 3 to 30 nt, e.g., from 10-30 nt, 15 to 30 nt, and including from 20 to 30 nt. As its name implies, the target binding domain is designed to bind to its corresponding target under hybridization conditions. In general, the target binding domain is substantially complementary to a selected sequence of its corresponding target polynucleotide and provides sequence-specific binding to the target (e.g., through canonical (Watson-Crick) base pairing). Partial complementarity between the target binding domain and the target sequence is also allowed, with up to 4 non-canonical base-pairs or mismatches per 24 bp segment (e.g., 1 mismatch per 12; 2 per 16; 3 per 20, etc.) if such mismatches do not degrade the sequence-specificity of the probe-target binding. In fact, mismatches can in some cases improve the sequence-specificity of hybridization (see, e.g., BACKGROUND AND RELATED ART section above).

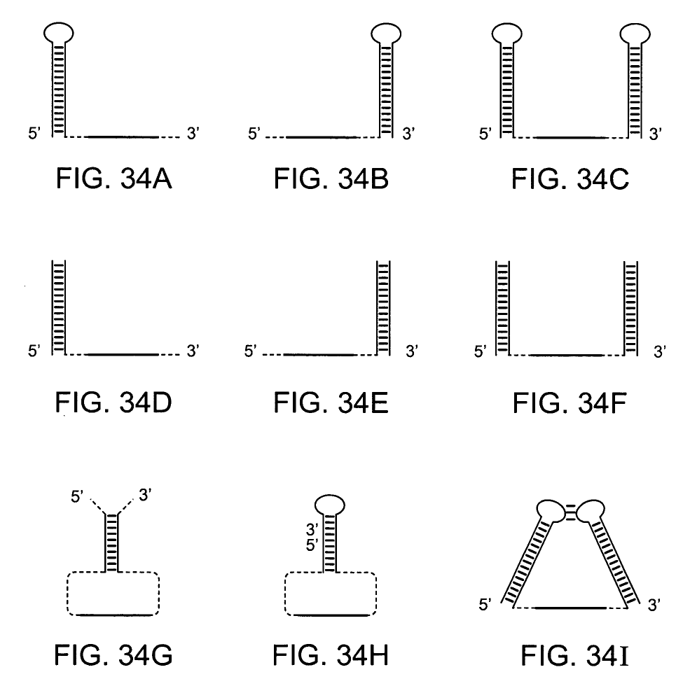

The binding enhancer domain includes additional polynucleotide sequences that do not form stable complexes with the target binding domain or its corresponding target polynucleotide. In certain embodiments, the binding enhancer domain does not contain sequences complementary to the corresponding target, whereas in other embodiments, the binding enhancer domain may include regions that have substantial complementarity to the target, as long as these regions of substantial complementarity do not form stable complexes under hybridization conditions. In certain embodiments, the binding enhancer domain ranges from 30 to 10,000 nucleotides in length. The binding enhancer domain may be placed at either end of the target binding domain or, in certain embodiments, be formed from sequences that are both upstream (5′) and downstream (3′) of the target binding domain, e.g., surrounding the target binding domain. In certain embodiments, more than one binding enhancer domain is employed in a polynucleotide probe. In such embodiments, the binding enhancer domains may be located 5′ of the target binding domain, 3′ of the target binding domain, or both 5′ and 3′ of the target binding domain (see FIG. 34).

In certain embodiments, the binding enhancer domain includes certain features that can be used either simultaneously (in concert) or in different combinations depending on what signal generation and detection methods are combined with the probes. For example, it is known that when an antisense RNA sequence is placed adjacent to certain non-antisense sequences, the antisense RNA sequence can be harder to displace from its complex with complementary target RNA than an antisense sequence that is not adjacent to non-antisense sequences (Homman et al. 1996). The “binding enhancer domains” in polynucleotide probes of the present invention, when linked to a target binding domain (sometimes referred to as an anti-sense sequence), promote more efficient binding between the target binding domain and the corresponding RNA target than the target binding domain would have alone. (see EXAMPLES).

In certain embodiments, a binding enhancer domain includes regions having predetermined intramolecular (e.g., in polynucleotide probes having a single nucleotide strand) or intermolecular (e.g., in bi-partite probes) secondary and/or tertiary structure, e.g., stem-loop structures, pseudoknots, bi-partite nucleic acid duplexes, nucleic acid triplexes and nucleic acid tetraplexes. In certain embodiments, the predetermined secondary or tertiary structure includes one or more sequences substantially related to a catalytically active hairpin ribozyme, a catalytically inactive hairpin ribozyme, a truncated hairpin ribozyme, a tRNA, or a region from a ribosomal RNA. As shown in the Examples section below, addition of these structured binding enhancer domains to the probe provides both better probe-target binding (in terms of hybridization kinetics and affinity), and higher signal-to-noise ratio (see EXAMPLES).

The present invention describes new stringency elements provided by inclusion of sequences derived from the hairpin ribozyme in the binding enhancer domain (see below). In addition to increased sequence-specificity, the hairpin ribozyme feature can also allow the probe to undergo reversible self-circularization, thereby further increasing the sequence specificity of the circular probes and enhancing probe-target affinity and allowing the option of signal amplification via rolling circle amplification (RCA; see below).

Accordingly, binding enhancer domains confer improved hybridization characteristics upon the hybridization of a target binding domain to a target polynucleotide. Such improvements in hybridization characteristics include improvements in selectivity (sequence-specificity), sensitivity (signal-to-noise ratio), affinity and binding efficacy.

In certain embodiments, the binding enhancer domain is located 5′ of the target binding domain. In some embodiments, it is located 3′ of the target binding domain. In some embodiments, the binding enhancer domain is divided, such that one subdomain is located 5′ of the target binding domain and another subdomain is located 3′ of the target binding domain.

An additional feature provided by the non-antisense sequence is the ability to carry more labels (e.g., radioactive or fluorescent) or modified nucleotides that can be used for detection without compromising the probe-target binding. Ordinary long probes can also carrying multiple labels, but unlike probes of the present invention, they also provide increased background noise and false-positive signals due to the ease of forming complementary complexes with non-target sequences (see BACKGROUND AND RELATED ART).

Yet another additional feature provided by the non-antisense sequences is the ability to provide probe signal amplification in a multiplexed format. The signal amplification can be performed using formats known in the art, including primer extension, sandwich hybridization, PCR, RT-PCR and RCA (see BACKGROUND AND RELATED ART). For these purpose, the probe non-antisense sequences are designed to provide simultaneous detection of multiple different probes under a uniform assay condition. The non-antisense sequences may vary for different probe-target pairs, or alternatively they may be target-independent (universal) sequences. In certain embodiments, the non-antisense sequence contains both universal sequence and target-specific sequences (e.g., Zip-code, ID or Tag sequences; see BACKGROUND AND RELATED ART) that can form duplexes with similar Tₘ and therefore lend themselves to multiplex detection (see below).

### Targets

Polynucleotide probes of the current invention were designed to detect any type of polynucleotide target, including single stranded RNA and DNA. Species of single stranded RNA include but are not limited to mRNA, ribosomal RNA, non-coding RNA and viral genomic RNA. Species of single stranded DNA include but are not limited to denatured genomic DNA, denatured viral DNA, and denatured bacterial DNA. The target polynucleotide can be in linear or circular form. Target circularization can be achieved by methods known in the art.

The targets to be detected can be obtained directly from natural sources or amplified from natural sources by methods such as PCR or RT-PCR, or by transcription-based or other isothermal amplification techniques.

Naturally-existing targets can be assayed directly in cell lysates, in nucleic acid extracts, or after partial purification of fractions of nucleic acids so that they are enriched in targets of interest.

The polynucleotide target to be detected can be unmodified or modified. Useful modifications include, without limitation, radioactive and fluorescent labels as well as anchor ligands such as biotin or digoxigenin. The modification(s) can be placed internally or at either the 5′ or 3′ end of the targets. Target modification can be carried out post-synthetically, ether by chemical or enzymatic reaction such as ligation or polymerase-assisted extension. Alternatively, the internal labels and anchor ligands can be incorporated into an amplified target or its complement directly during enzymatic polymerization reactions using small amounts of modified NTPs as substrates.

### Methods

The polynucleotide probes described herein can substitute for ordinary probes in commonly used hybridization methods such as dot/slot blots, Northern and Southern blots, in situ hybridization, sandwich hybridization, and gel-shift assays. These probes allow faster, more accurate, and more sensitive detection and quantification of target polynucleotides with a higher level of multiplexing than ordinary hybridization probe. In particular, RNA Lasso probes provide much stronger shift of target mobility in electrophoretic mobility-shift assays (EMSA) allowing easier detection of the presence of the target by this method (see EXAMPLES).

In addition to the assays above, we provide new methods for quantitative detection of polynucleotide targets that accommodate a number of salient features of these new probes. Exemplary methods include the following steps:

a) hybridizing a target polynucleotide to a probe according to the present invention;

b) isolating the target-probe hybridization complexes under stringent hybridization conditions (e.g., to remove non-specifically bound complexes);

c) recovering the polynucleotide probe from the hybridization complexes;

d) hybridizing the recovered polynucleotide probe from step (c) to a probe-specific synthesis primer;

e) placing the probe-specific synthesis primer-hybridized probe under nucleic acid synthesis conditions to extend the probe-specific synthesis primer; and

f) detecting the extended probe-specific synthesis primer.

In one embodiment, the binding between the probe and polynucleotide target is carried out in solution and the target detected with a gel-shift assay using labeled probes or labeled targets (see EXAMPLES).

In another embodiment, the binding between the polynucleotide probes and their targets is followed by immobilization of the formed complex on a solid support. In yet another embodiment, the polynucleotide target to be detected is immobilized on a solid support before binding to the probe. Polynucleotides can be immobilized by various methods known in the art including, (without limitation) covalent cross-linking to a surface (e.g., photochemically or chemically), non-covalent attachment to the surface through the interaction of an anchor ligand with a corresponding receptor protein (e.g. biotin-streptavidin or digoxigenin-anti-digoxigenin antibody), or through hybridization to an anchor nucleic acid or nucleic acid analog. The anchor nucleic acid or nucleic acid analog (see BACKGROUND AND RELATED ART) have sufficient complementarity to the target (i.e., their formed duplex has sufficiently high Tₘ) that the anchor-target-probe complex will survive stringent washing to remove unbound targets and probes, but they do not overlap with the target site that is complementary to the probe antisense sequence. The stringent washing step is followed by release of the bound probes under denaturing conditions and their subsequent recovery.

In one embodiment, the covalently immobilized targets have at least one link to a surface per polynucleotide molecule.

In another embodiment, which is specific for self-circularizable probes capable of forming topologically-linked complexes, the covalently immobilized targets have at least two links to the surface per polynucleotide molecule. In this case, it is desirable that the probe binding site be located between these two links. If the target is circular, it needs just one (but could have more than one) link to the surface to form a topologically-linked complex with circularizable probes. In both cases, the topologically-linked probe-target complexes can be washed under fully denaturing conditions to minimize background noise. After washing, the circularizable probes can be self-cleaved and released from the target-probe complex under denaturing conditions.

A variety of surfaces known in the art can be used for immobilization of the target or the probe-target complex, including but not limited to glass, plastic, nylon, or nitrocellulose, with or without additional surface functionalization. These materials can be in various formats, including without limitation synthetic or natural beads, membranes or filters, slides including microarray slides, microtiter plates, microcapillaries, and microcentrifuge tubes.

The recovered target-specific probes represent the targets for which they are specific, and by quantifying them one obtains information about the abundance of those targets. In one embodiment, the recovered probes are directly quantified in solution by qPCR using RT and PCR primers that are specific for either universal (target-independent) or target-specific, Zip-code/ID sequences contained in the probe.

In another embodiment, the polynucleotide probes recovered from the specific complexes with their targets are directly cloned and fast-sequenced by sequencing techniques such as SOLiD (Applied Biosystems,), 454 (Roche) and Solexa (Illumina).

In another embodiment, the recovered probes are hybridized to tethered oligonucleotide primers prior to amplification as in the first embodiment. The tethering is accomplished by the chemical attachment of the primers through their modified 5′-ends, using methods known in the art, to solid supports such as microtiter plate wells, or beads, or slides. In certain embodiments, “single-plex” formats are employed whereas in certain other embodiments, a “multiplex” format is employed. A number of multiplex detection formats can be used, including either labeled/tagged bead sets (e.g., those produced by Luminex), in which each label is assigned to the individual probe-specific primer, or oligonucleotide arrays on slides, in which in which specific oligonucleotide spot/position is assigned to the individual probe-specific primer. The limited sequence complexity of the recovered target-specific probes provides conditions for easier and higher level multiplexing, especially using with universal and Zip-code/ID sequence tags.

After the hybridization of the tethered primers to the probes, the primers are extended by a nucleotide polymerase. In certain embodiments, the polymerase is selected from an RNA polymerase and a reverse transcriptase.

In one embodiment, the primer extension is performed in the presence of modified NTPs to create a DNA tag containing anchor ligands (see above). The probe is then degraded or washed away, and the DNA tag attached to the solid support is detected by ELISA methods known in the art, e.g., using enzyme-linked protein, antibodies and aptamers and chemiluminescence. Alternatively, 3-D DNA/Dendrimers signal amplification technology (Genisphere) can be used for high-sensitivity ELISA detection.

In another embodiment, the primer extension creates DNA/RNA tag sequences that comprise universal (target-independent) and optional target-dependent Zip-code/ID sequences. After the probe is degraded or washed away, the DNA tag sequence attached to the solid support can be detected and quantified by one of the standard signal amplification methods. The probe design determines the choice of detection method and vice versa. For example, the universal tag sequence can be hybridized with universal linker oligonucleotides and detected by branched-DNA (bDNA) technology (see BACKGROUND AND RELATED ART).

In yet another embodiment, in case of self-circularizable probes, rolling circle polymerization can provide signal amplification without the need to wash or destroy probes bound to the tethered primers.

### Kits

Also provided by the subject invention are kits that include the hybridization probes of the invention for use in various applications, as described above and in the Examples section below. For example, kits according to the present invention may include hybridization probes attached to a solid support, e.g., on a bead or in an array format (e.g., a microarray as is employed in the art) as well as reagents for performing hybridization assays. Kits may also include probe-specific synthesis primers, e.g., in the form of an addressable array on a solid support. The kits may also include reagents for performing control hybridizations (e.g., control targets and/or control hybridization probes) and instructions for using the hybridization probes in hybridization assays.

### Utility

The polynucleotide probes of the present invention provide several advantages. First, the probes provide a superior combination of efficiency, sensitivity and specificity of hybridization: they provide for hybridization that exceeds the sequence-specificity of ordinary short oligonucleotide probes, that exceed the kinetics of ordinary long polynucleotide probes, that exceeds the affinity to polynucleotide and oligonucleotide target, and that exceeds the sensitivity (signal-to-noise ratio) of ordinary short and long polynucleotide probes. Furthermore, they are SNP capable, they can carry multiple signal-reporting labels or ligands, and they provide for a higher level of multiplexing of diverse target sequences.

Second, the polynucleotide probes of the present invention can hybridize quickly and efficiently with a variety of polynucleotide targets. Such targets include single-stranded RNA and DNA polynucleotides (such as mRNA, cRNA, cDNA, non-coding RNA, miRNA precursors) even if they contain extensive secondary structure; denatured double-stranded DNA (such as genomic DNA, PCR gene amplicons); and short RNA (such as miRNA or siRNA).

Third, because the probes of the present invention may be designed to carry universal target-independent sequences for PCR primers and/or transcription promoters, they are amplifiable. Such a benefit is useful for automated selection of effective antisense sequences under multiplex assay conditions for simultaneous detection of different targets; for target-dependent signal amplification by RT-PCR and in vitro transcription; and for cloning and fast sequencing after PCR amplification. In addition, the universal, target independent sequences can carry restriction sites for fast direct cloning and sequencing. Finally, amplication provides an opportunity for labeling the amplied probes with modified NTPs such as radiolabels, fluorescent tags, biotin, digoxigenin.

Fourth, the probes of the present invention can be easily prepared by in vitro transcription from appropriate DNA template. The cost of such preparation is comparable to that of ordinary RNA probes prepared for Northern blots and in situ hybridization. Furthermore, the probes can be labeled (if necessary) during the preparation by transcription using modified NTPs such as radiolabels, fluorescent tags, biotin, and digoxigenin. Polynucleotide probes of the present invention can be used in place of hybridization probes that are routinely used in gel-shift assays, e.g. capillary electrophoresis and microfluidic chips; sandwich-hybridization assays, e.g. single-plex assays in microtiter plates and multiplex assays on coded beads or arrays; gene arrays featuring immobilized targets and probes in solution, e.g. dot-blots, slide-based arrays of spotted targets, coded-bead arrays, in situ hybridization, and tissue arrays; and Northern blots.

As demonstrated in the Examples provided below, polynucleotide probes of the instant invention are more efficient and more sequence specific than ordinary antisense probes (both RNA and DNA). In efforts to understand possible mechanisms of such enhancements, we studied probes that were derived from Lassos as well as probes unrelated to Lassos. The results shown in the Examples herein indicate that polynucleotide probe superiority is not restricted to specific Lasso structures but rather involves a general role for non-complementary sequences/structures attached to short antisense sequences that are complementary to a polynucleotide target.

### EXAMPLES

### Example 1

### RNA Lasso Probes

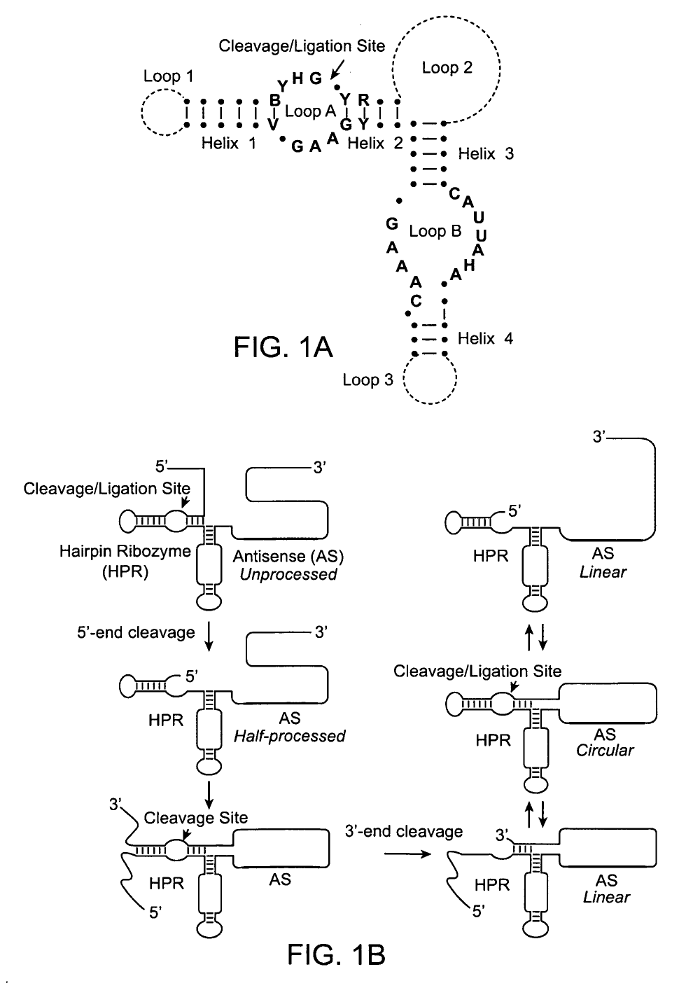

RNA Lasso™ are a proprietary class of RNA molecules developed by SomaGenics, Inc., that can hybridize to and circularize around polynucleotide targets (Johnston et al. 1998; Kazakov et al. 2004). RNA Lassos differ from DNA Padlock probes, which are another type of circularizable nucleic acid probe (see BACKGROUND AND RELATED ART). Lassos are 110-150 nt RNAs that can be transcribed in vitro and used without purification whereas the Padlock probes are 70-110 nt long chemically synthesized oligodeoxynucleotides that must be carefully purified to allow target-dependent ligation of their ends by a DNA ligase on the cDNA template. Since the topologically linked Padlock probes cannot be efficiently amplified by DNA polymerases, they must be displaced from the target or linearized by restriction enzyme cleavage assisted by oligodeoxynucleotide splint (Hardenbol et al. 2003). In contrast to DNA Padlock probes, RNA Lassos do not require protein enzymes for circularization and cleavage; instead, they use an internal hairpin ribozyme (HPR) domain (FIG. 1A) that has both self-cleavage and self-ligating properties (Fedor, 2000). Any one of loops 1-3 of the HPR can be either deleted or used for insertion of additional (functional) sequences (Feldstein & Bruening, 1993; Komatsu et al. 1993, 1995; Berzal-Herranz & Burke, 1997; Kisich et a1.1999).

The original Lasso design uses loop 2 for insertion of antisense and bridging segments. Self-processing (cleavage and ligation) of the Lasso allows it to excise itself from a primary transcript, cleaving off all extraneous flanking sequences at both 5′- and 3′-ends, and then to undergo circularization via intramolecular ligation (FIG. 1B). We reasoned that attaching an antisense sequence adjacent to the ribozyme core would allow it to pair with a target mRNA, intertwining the two RNAs, and then undergo self-ligation creating a-topologically linked complex with a linear target mRNA. In contrast to the conventional application of ribozymes as sequence-specific nucleases, Lassos do not cleave their targets but topologically encircle them creating complexes that are more thermodynamically stable than ordinary RNA-RNA duplexes.

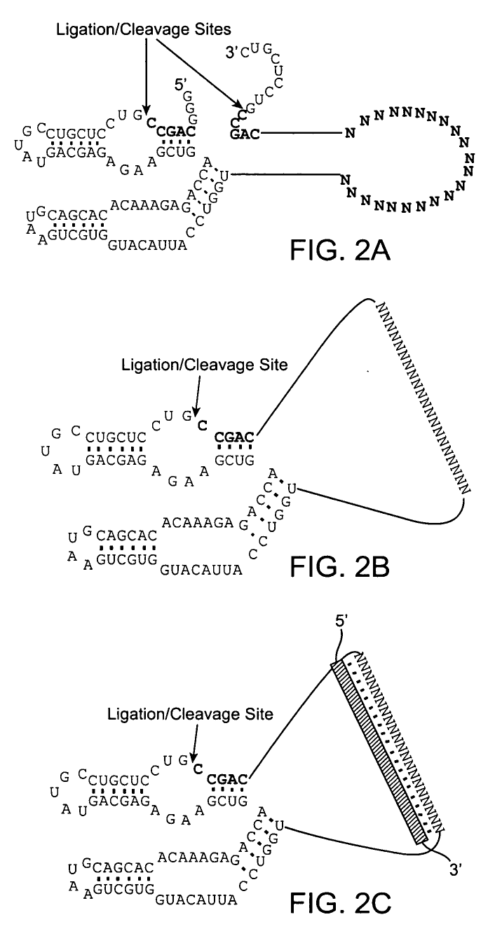

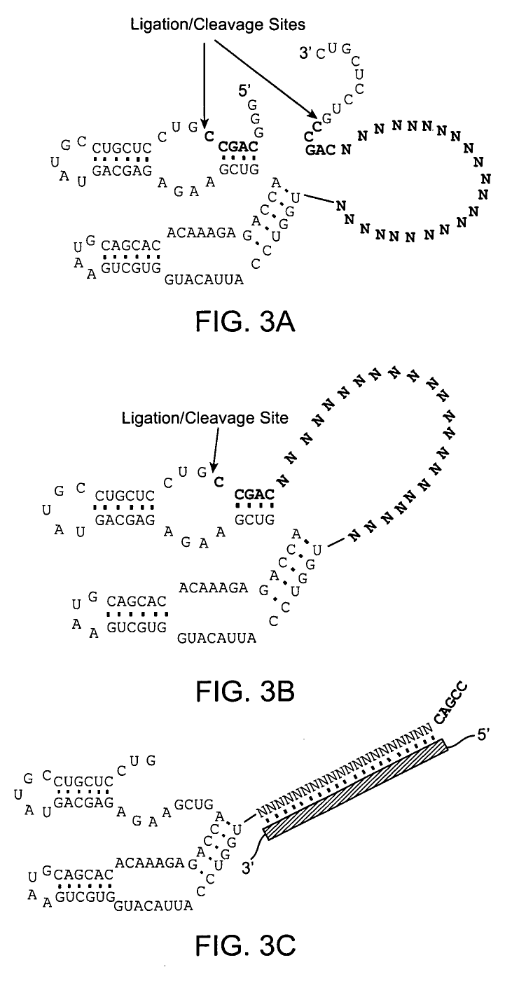

There are two Lasso formats: Lasso I (FIG. 2) and Lasso II (FIG. 3). Both Lassos can sequence-specifically hybridize to polynucleotide targets but, in contrast to Lasso II, Lasso I cannot form topological linkage (circularize around target).

### Example 2

### Lasso Self-Processing, Circularization and Target Binding

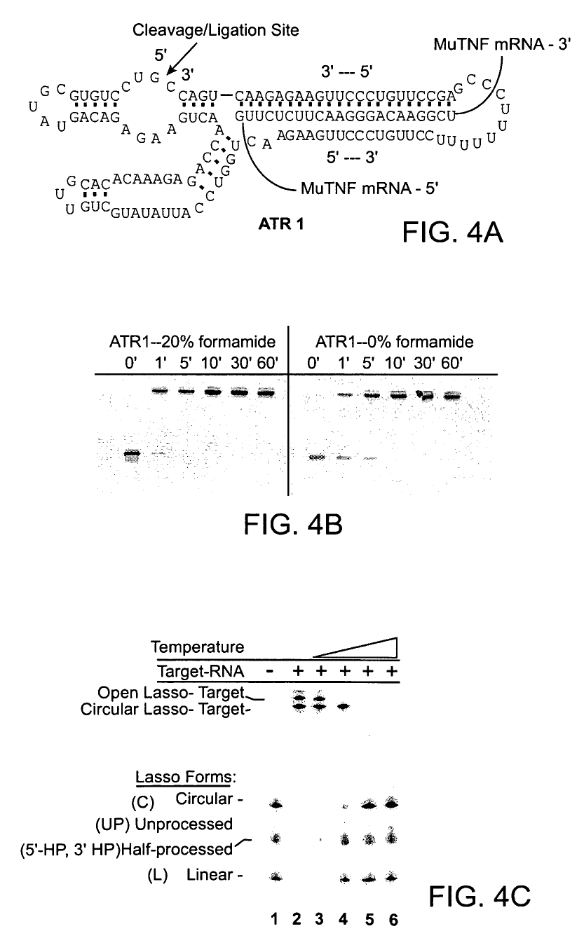

An RNA Lasso, designated ATR1, targeting a site in the coding region of mouse tumor necrosis factor alpha (TNFα) mRNA (FIG. 4A), was transcribed from a DNA template by T7 RNA polymerase. Self-processing of the 133-nt primary transcript resulted in half- and fully-processed linear (L) species as well as the covalently closed circular (C) form (FIGS. 1B and 4C, lane 1). The relative electrophoretic mobilities of the L and C forms correspond to a known feature of RNA molecules: circular forms migrate in denaturing PAGE more slowly than do their linear counterparts (Feldstein & Bruening, 1993).

Binding of ATR1 transcripts with RNA targets has been tested in gel-shift assays. Both linear and circular species of ATR1 form with target RNA unusually strong complexes that are stable enough to be detected by denaturing PAGE (FIGS. 4B and 4C, lanes 2-3). In time course experiments, we have shown that Lasso hybridization with target RNA is fast (FIG. 4B). In buffer that lacks formamide, over 50% of ATR1 Lasso was bound within 1 min. In buffer that contains 20% formamide, the binding reaction is even faster, with virtually all targets bound within 1 min (FIG. 4B).

### Example 3

### Both Linear and Circular Lassos Efficiently Bind to Target RNA

³²P-labeled ATR1 was incubated with excess TNF-709 target RNA, and products were analyzed by 6% denaturing PAGE. When Mg²⁺ was included in the incubation buffer, the circular and linear species could easily interconvert and both Lasso species were able to form a strong complex with target RNA, seen as two distinct Lasso-target complexes on the gel (FIG. 4C, lanes 1-2). In EDTA-containing buffer, lacking the Mg²⁺ required for HPR cleavage and ligation, the circular and linear forms could not interconvert, and only linear species were able to form the strong complex with target RNA (data not shown). These results indicate that the circular Lasso can reversibly cleave itself in the presence of Mg²⁺ before or after hybridizing to target RNA, and then ligate again when bound to target RNA.

High concentrations of monovalent cations (˜1 M and above) can substitute for divalent metal ions (M²⁺) in supporting the catalytic activity of HPR (Nesbitt et al. 1997; Murray et al. 1998) that is responsible for Lasso self-processing and circularization. We confirmed that Lassos can efficiently perform both self-cleavage and ligation reactions as well as binding to target RNA under high salt (but Mg²⁺-free) buffer conditions that are commonly used for dot-blot hybridization assays on solid supports such as Church hybridization buffer (0.5M sodium phosphate pH 7.2, 1 mM sodium EDTA, and 7% sodium dodecyl sulfate) (Church & Gilbert, 1984), which has an estimated Na⁺ concentration of 1.1 M (data not shown).

### Example 4

### Lassos can Form True Topologically Linked Complexes Through Circularization Around a Target

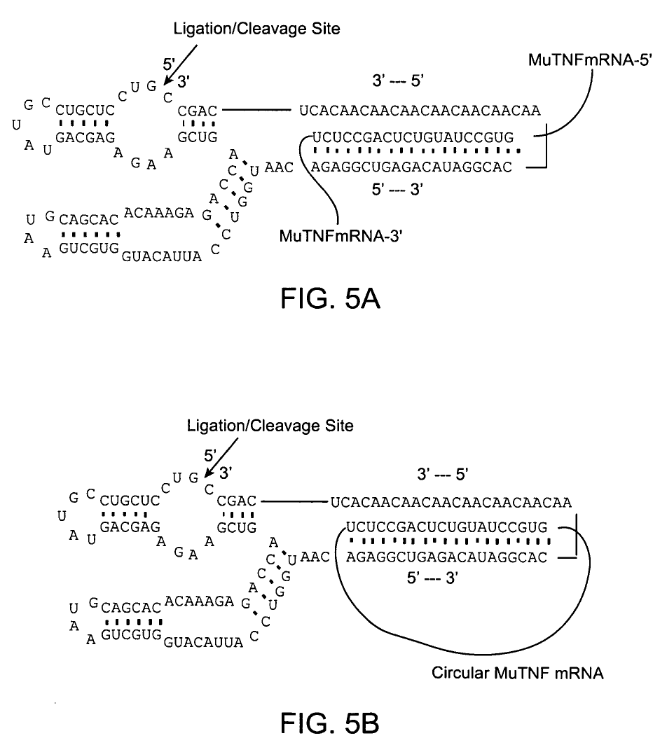

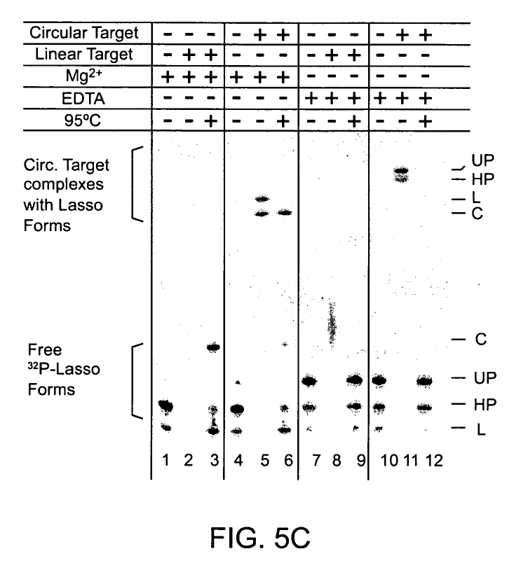

To prove that Lasso circularization upon binding to an RNA target does, in fact, result in topological linkage, we analyzed binding of Lasso 229-7.0, which targets the 229-248 nt site of TNFα mRNA, with the corresponding linear and circular model targets (FIGS. 5A-B) by 6% denaturing PAGE (FIG. 5C). The 120 nt linear RNA target containing the appropriate TNFA sequence was circularized using the method described by Beaudry & Perreault, 1995. ³²P-labeled Lasso 229-7.0 was incubated with linear and circular targets, respectively, under conditions where Lassos can or cannot self-process—i.e., in the presence or absence of free Mg²⁺ (FIG. 5C, lanes 1-6 and 7-12, respectively).

When the Lasso was incubated with circular target RNA in the presence of 10 mM Mg²⁺, three discrete gel-shifted bands were observed (FIG. 5C, lane 5), whereas when it was incubated with the linear target in either buffer, complexes dissociated during electrophoresis and were visible only as a smear (FIG. 5C, lanes 2 and 8). Upon incubation of 229-7.0 with the circular target in EDTA-containing buffer that renders the Lasso catalytically inactive, two higher mobility complexes were observed. The identity of each band was assigned by gel shift analysis before and after highly denaturing treatment at 95° C. for 2 min. For the EDTA-containing reactions with circular target, the dissociated Lasso species correlate with the unprocessed and half-processed Lasso species (FIG. 5C, lanes 11-12). For the Mg²⁺-containing buffer, the two upper gel-shifted bands were mostly dissociated upon incubation at high temperature and correlate with the reappearance of fully processed and half-processed linear forms of the Lassos, respectively. For the Mg²⁺-containing buffer, the upper shifted band disappeared upon heating, whereas the lower-shifted band survived even prolonged (for up to 10 min) incubation at 95° C. (FIG. 5C, lanes 5 and 6). Since a circular Lasso band was not seen as a product of dissociation, we concluded that the surviving band represented a topologically linked complex between a circular Lasso and circular target.

### Example 5

### Selection of Lassos Capable of Binding Fast to and Circularizing Around Target RNA in Solution

The efficacy and specificity of hybridization between probes and targets depends on target site accessibility, hybridization rate and duplex stability (Sohail & Southern, 2000; Sczakiel & Far, 2002). Rational design of probe sequences based on experimental and theoretical considerations have had only limited success (Sohail et al. 2001; Sczakiel & Far, 2002). As an alternative, several methods for selecting efficiently hybridizing sequences from nucleic acid libraries have been developed (Rittner et al. 1993; Stull et al. 1996; Bruice & Lima 1997; Kronenwett & Sczakiel 1997; Lima et al. 1997; Milner et al. 1997; Ho et al. 1998; Patzel & Sczakiel, 2000; Allawi et al. 2001; Lloyd et a1.2001; Pan et al. 2001; Scherr et al. 2001; Liang et al. 2002). We have developed an in vitro selection method to find Lassos that can efficiently bind to and circularize around the target. This method has been used to select optimal Lasso probes for several targets, including TNFα and the HCV-IRES (Kazakov et al. 2005), starting with Lasso libraries that contain all possible 20-22 nt antisense sequences for the target gene of interest. First, we describe our method for generating these gene-specific, or directed libraries (Seyhan et al. 2005), using the target TNFα as an example.

For purpose of preparation of gene-specific Lasso libraries, sense and antisense strands of TNFα mRNA are produced by separate in vitro transcription reactions from a PCR template encoding the murine TNFα gene flanked by opposing T7 and SP6 promoters. Double stranded RNA is formed by annealing the resulting complementary RNAs as described in Kawasaki et al. (2003). Next, the dsRNA is digested by recombinant Dicer ribonuclease (Stratagene) or bacterial RNase III and the resulting 20-22 bp dsRNA products are gel-purified and dephosphorylated with alkaline phosphatase (Promega). In two ligation steps, flanking oligonucleotides encoding primer-binding sites for subsequent PCR amplification were attached to the 3′- and 5′-ends of each fragment by T4 RNA ligase (Promega). Finally, the products from the second ligation reaction were amplified by RT-PCR. The resulting gene-specific DNA fragments were cloned and sequenced. 15 of 18 of the sampled sequences were perfect matches with TNFα mRNA sites while the 3 other sequences contained single-nucleotide mismatches or deletions that were most likely introduced into the library during PCR by Taq polymerase. To incorporate these gene-specific oligonucleotide sequences into Lasso-expression DNA templates, the sequences of the PCR primers described above were designed to partially overlap with invariant Lasso sequences. The Lasso transcription templates were then synthesized by PCR cloning using two extra PCR primers encoding the rest of the Lasso sequences and a T7 promoter. This TNF-specific Lasso library was transcribed in vitro with T7 RNA polymerase (Ambion) to generate the initial pool of Lassos for in vitro selection (see section C3.2). We confirmed that the transcribed library contains active Lasso species that can self-process and circularize (data not shown).

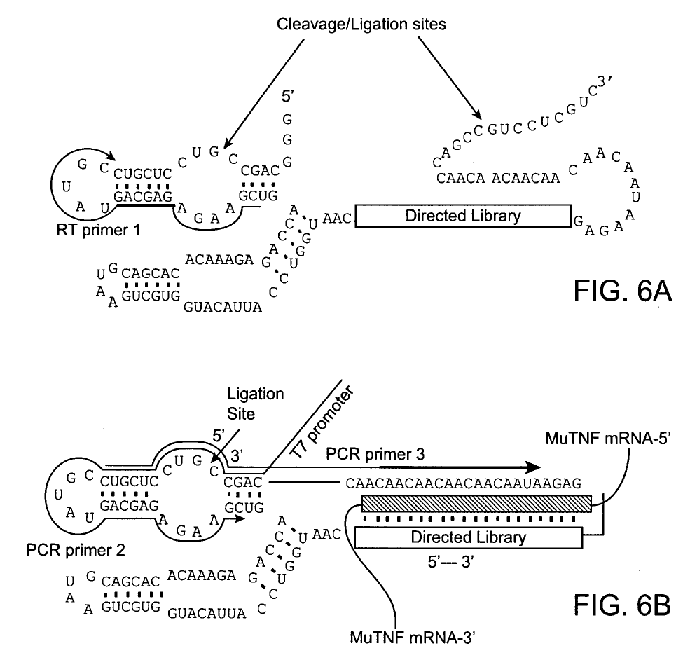

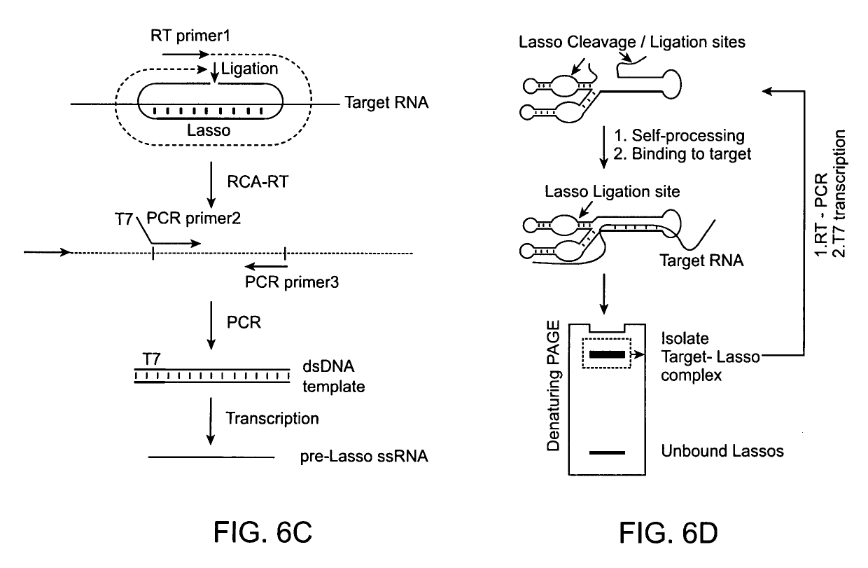

The scheme for selecting Lassos that are especially effective at binding to and circularizing around a given target is shown in FIG. 6. We developed a method for selectively amplifying circular Lassos that were topologically linked to their target mRNA by using a primer designed to reverse transcribe only circular molecules (RT primer 1). This primer is annealed to the unique, complementary sequence near the 5′-end of the Lasso RNA (FIG. 6A). The primer hybridizes across the active site of the HPR domain, and prevents further self-processing of the Lasso during subsequent manipulations. A reverse transcription (RT) reaction yields (via a rolling circle mechanism) single-stranded DNA multimers of the Lasso sequence (FIG. 6B-C). Linear (unligated) Lassos yield only a short abortive product, which will not be amplified by PCR in the next step. Second, two additional primers (T7 PCR primer 2 and PCR primer 3) are used to PCR-amplify the rolling circle RT product and add the T7 promoter sequence. Finally, the optimized Lassos were transcribed from these PCR products using T7 RNA polymerase (FIG. 6B-C). This technique was initially tested on the free Lasso in the absence of its target (data not shown). As the next step toward implementing the Lasso selection scheme, we confirmed that we were able to amplify circular Lassos bound to target RNAs that were purified by the gel-shift assay using 6% denaturing PAGE (FIG. 6D). The band corresponding to circular Lasso-target complex was excised and eluted from the gel and used in the RCA-RT-PCR protocol described above.

Using this method, we selected Lasso species that are capable of rapid binding and circularizing around TNFα mRNA starting from a complete TNF-specific Lasso library. For this selection, we used a 1000-nt fragment of murine TNFα mRNA (TNF-1000) that contains the 5′-UTR (untranslated region), the entire coding region, and over 100 nucleotides of the 3′-UTR.

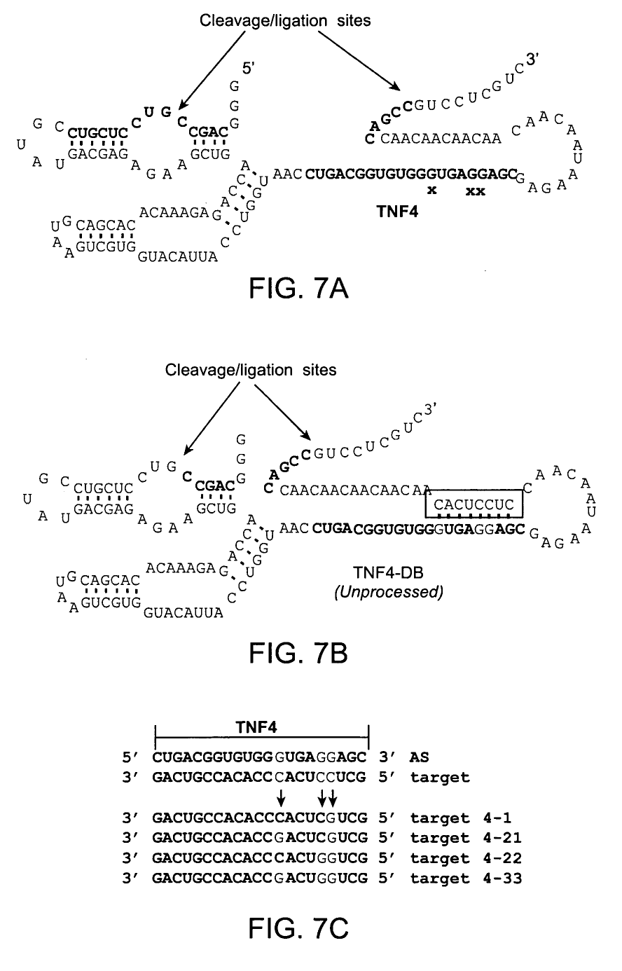

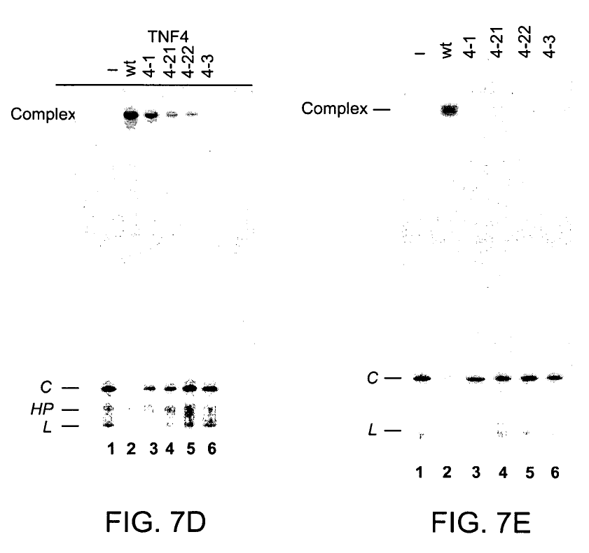

Three rounds of selection were performed as shown in FIG. 6B-D. For the initial round of selection, 400 pmol of the Lasso directed library were incubated with an excess of TNF-1000 target at 37° C. for 60 min in SB buffer containing 10 mM MgCl₂, 50 mM Tris-HCl (pH 7.5) and 20% formamide. These conditions ensure that the library complexity is retained through the initial round of selection. Reactions were analyzed on a denaturing 6% PA gel to separate free Lasso from the Lasso-target complex. RNA was visualized in the gel by ethidium bromide staining. Complexes were excised and eluted from gel slices and amplified by RT-PCR as described above. The RT-PCR product was gel-purified on a 1.5% agarose gel and extracted using the QIAquick Gel Extraction Kit (Qiagen). The resulting DNA was used as the transcription template to generate the enriched Lasso library for the next round of selection. The entire selection cycle was repeated twice, decreasing in incubation time to 30 min for round 2 and 5 min for round 3 to favor Lassos that hybridize quickly. After the third round of selection, the gel-purified RT-PCR fragment was cloned using the TA-cloning kit (Invitrogen). The resulting colonies were screened for inserts by blue/white color selection. 23 individual clones were isolated and sequenced to identify the selected antisense sequences. As expected from the directed library contents, all Lasso antisense sequences ranged from 20-22 nucleotides. Among the identified sense (target) sequences, we found the most frequently selected site (“hot-spot”) was located in the target region between nucleotides 589 and 619. To confirm that the selected Lassos are superior binders, one representative clone of each unique selected sequence was transcribed in vitro and tested in binding affinity and kinetics assays. Lassos were internally ³²P-labeled during in vitro transcription and incubated with an excess of non-radioactive target TNF-1000 RNA at 37° C. in the SB buffer (see above). Products of these reactions were analyzed by denaturing 5% PAGE. From this additional screen, the selected Lasso TNF4 (FIG. 7A) was identified as one of the fastest binders (t_{1/2}˜1 min) in addition to being able to circularize around target efficiently (FIG. 7D, lanes 1-2).

### Example 6

### Sequence-Specificity and SNP-Discrimination of Lasso Binding to Target in Solution

One could argue that Lassos should be less capable of discriminating target sequences because of the high-stability of Lasso-target complexes. However, our data demonstrate that Lassos are in fact highly target-specific. No complex formation was observed by our standard gel-shift assay when Lassos designed to bind to certain sites in the TNF target were incubated with an unrelated RNA target, i.e. DsRed mRNA and fragments of TNFα mRNA that do not have the Lasso-specified site. Conversely, the Lassos selected to target other genes that are not complementary to TNF do not bind this target (data not shown).

We also demonstrated that Lasso probes are able to specifically bind their targets in a complex mixture of RNAs such as cellular RNA, where the number of non-target RNAs vastly outnumbers the desired target RNA for each individual probe. Specifically, we assayed 4 different selected anti-TNF Lassos with different amounts of TNF target RNA in solutions containing an excess of total cellular RNA extracted from 293FT cells. Using our standard gel-shift assay we showed that the efficacy and specificity of strong complex formation for these Lassos was unaffected at various ratios of the TNF target to total RNA. In addition, we compared further the ability of Lasso TNF4 to hybridize to its target RNA alone with total RNA and with ever decreasing ratios of target RNA to total RNA. Even in the lowest target-to-total RNA ratio tested (1:230), Lasso TNF4 was able to bind target RNA virtually at the same efficacy as if total RNA was not added to the solution (data not shown).

Moreover, we tested the ability of the selected Lasso TNF4 to discriminate between perfectly matched and mismatched target RNAs. A series of mutated TNF targets containing no mismatch (wt), 1 mismatch (4-1), two nonadjacent mismatches (4-21), two adjacent mismatches (4-22), or 3 mismatches (4-3) out of 21 nt antisense in Lasso TNF4 (see FIG. 7C) were prepared using the Quick-Change Mutagenesis kit (Stratagene). PCR reactions designed to add a T7 promoter and transcribe a portion of the TNF sequence containing the mutated region were performed. The resulting PCR amplicon was used to transcribe the mutant TNFα target RNAs, which were the derivatives of TNF1000, for use in binding assays in vitro. ³²P-labeled Lassos were incubated with excess target RNAs under standard Lasso reaction conditions for 60 min and analyzed by gel-shift assay (FIG. 7D). TNF4 forms a strong complex with the perfectly matched targets (wt) as seen previously. While they both bind to singly mismatched target 4-1 efficiently (though not as well as to wt), strong complex formation is greatly reduced but still detectable for targets containing two (4-21 and 4-22) and virtually undetectable in the case of three mismatches (4-3). Similar to other antisense probes, the inherent specificity of a given Lasso should vary for different sequences and may be influenced by the GC-content, sequence and accessibility of the target sites among other factors.

Several designs of hybridization probes and antisense agents that improve mismatch discrimination have been suggested (see BACKGROUND AND RELATED ART). These schemes have in common some form of competition for the target-binding region of the probe. Like a molecular beacon, the Lasso can adopt two different conformations. In one conformation, the free, fully processed Lassos are equilibrating between circular and linear forms in solution, and energy is stored in helix 2 (see FIG. 1A). To bind to and circularize around a target, both circular and linear Lassos have to disrupt and re-form this helix, thus providing an element of competition. To increase the competition, we incorporated additional stringency elements into the selected Lassos and tested whether or not SNP-discrimination in the RNA targets was improved. One example is Lasso TNF4-DB, which is a derivative of TNF4 containing an internal pairing element in the antisense sequence that must be displaced to allow target binding (FIG. 7B). We showed that TNF4-DB discriminates the mismatched target sites (FIG. 7E), including the SNP (4-1), much better than its parent molecule, TNF4 (see FIG. 7D-E). Similar 7-9 nt “specificity modules” could be easily introduced into any given Lasso sequence selected first without the stringency element present.

### Example 7

### Lasso Probes Selected in Solution can also Efficiently and Specifically Bind to Immobilized RNA Targets

To test whether Lasso probes that were optimized in solution were also able to hybridize specifically to targets immobilized on a solid support, dot-blot assays were designed and performed. In these experiments, decreasing amounts of TNF1000 target RNA were mixed with a fixed amount of total cellular RNA (1 μg), which was extracted from human embryonic carcinoma cells NTERA-2cl.D1. The RNA mixtures were spotted on a positively charged nylon membrane (Immobilon-Ny+, Millipore) using a dot blot apparatus (Minifold 1, Schleicher & Schuell). The spotted RNA molecules were immobilized to the membrane by UV-cross-linking using a standard dot-blot procedure (120 milliJoule dose, UV Stratalinker 2400, Stratagene). An excess of internally ³²P-labeled TNF4 Lasso probe was incubated in Church buffer with the membrane-cross-linked target RNAs overnight at 37° C. in a hybridization incubator (Robbins Scientific). A series of washes of increasing stringency to remove unhybridized and linear Lasso probes from the membrane was performed as shown in Table 1.

TABLE 1
Wash stringency

| Wash Steps | Buffer | Temperature |
|---|---|---|
| 1 | 2x SSC, 1% SDS | 37° C. |
| 2 | 0.5x SSC, 0.1% SDS | 37° C. |
| 3 | 0.1x SSC*, 0.1% SDS, 1 mM EDTA | 37° C. |
| 4 | 0.1x SSC*, 0.1% SDS, 1 mM EDTA | 50° C. |
| 5 | 0.1x SSC*, 0.1% SDS, 1 mM EDTA | 58° C. |
| 6 | 0.1x SSC*, 0.1% SDS, 1 mM EDTA | 65° C. |

*0.1x SSC = 1.5 mM Na₃citrate, 15 mM NaCl

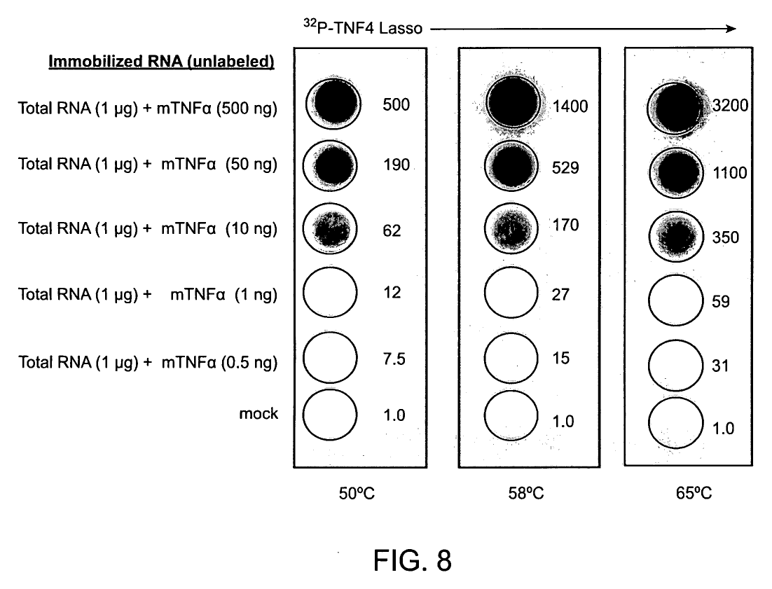

To track the progress of the wash steps, the membrane was removed from the hybridization tube after wash steps 4,5, and 6, imaged using a storage phosphor screen (Molecular Imager FX, Bio-Rad), and then replaced in the hybridization tube for the next wash step. Signal-to-noise ratios were calculated based on the background signal of the mock sample (S/N=1), and are shown along with corresponding dot images in FIG. 8. As can be seen, the Lasso hybridizes to the immobilized targets with low background, and the signal-to-noise improves as the stringency of washes is increased as a result of removal of Lasso probes bound non-specifically to the positively charged surface.

### Example 8

### LAMP Scheme of Detection and Quantification of RNA Targets Using RNA Lasso Probes

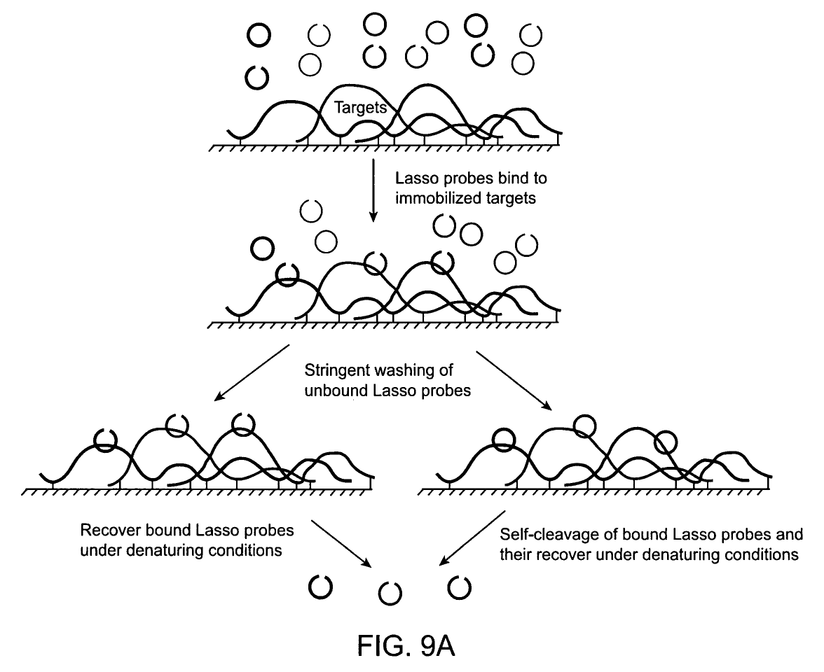

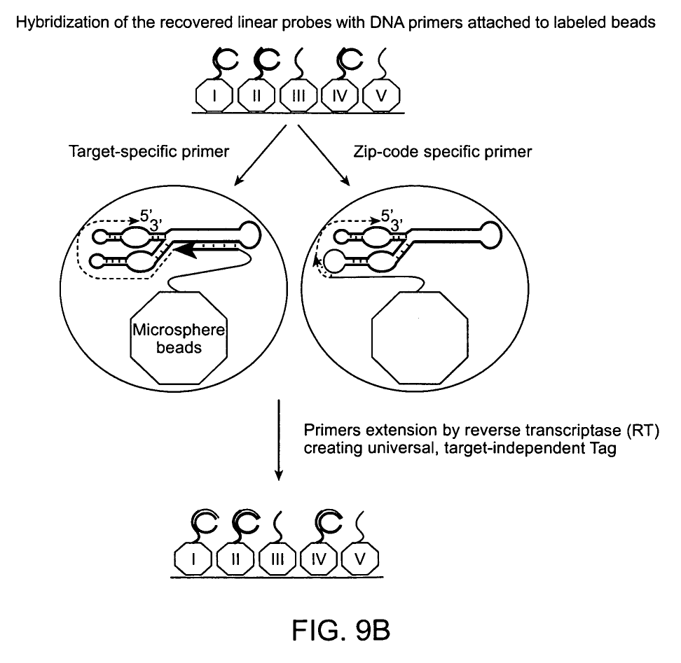

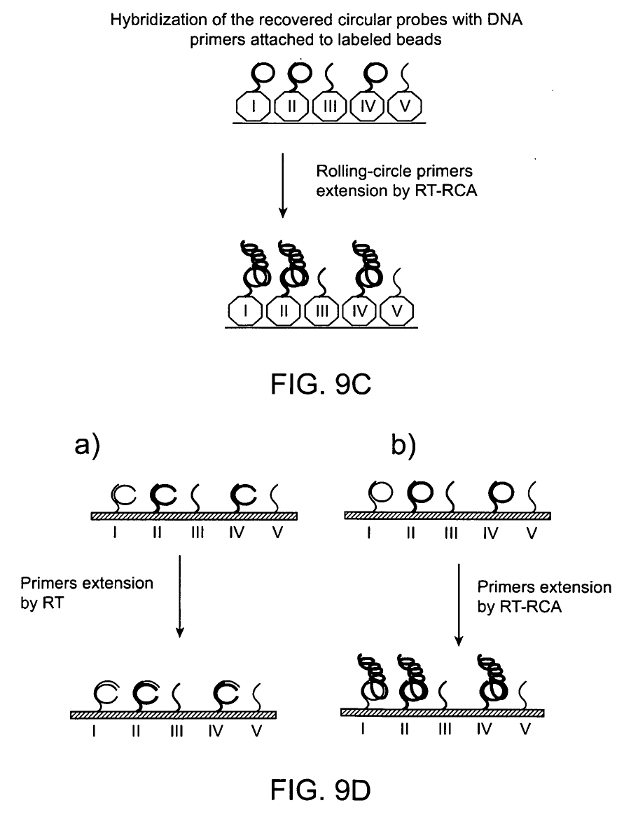

The LAMP (for Lasso multiplexing) technology involves the following steps illustrated in FIG. 9:

(1) Cellular RNA containing mRNA targets is immobilized (e.g., via chemical or photo-chemical cross-linking) to an appropriate surface (e.g., a nylon membrane).

(2) A mixture of target-specific RNA Lasso probes, added in excess, quickly hybridizes to and circularizes around the RNA strands of complementary targets, due to their internal ribozyme activity that can ligate their ends. These Lassos were designed to provide optimal sequence specificity and binding efficacy under the same hybridization and washing conditions for each target.

(3) Hybridized Lassos are washed under highly stringent conditions, whereupon only Lasso probes that can circularize around the targets will be retained, regardless of the GC-content of the hybridization site. The highly stringent washing decreases background noise associated with non-specific binding of probes to the surface.

(4) The retained circular Lassos is linearized by self-cleavage initiated by an appropriate change of ionic conditions, and then subjected to denaturing conditions that release the Lassos into the solution. The amount of recovered Lassos will correspond to the amount of RNA target present.

Steps 1 to 4 are schematically shown in FIG. 9A, which describes a capture of target-specific probes on immobilized targets and recovery of the captured probes.

(5) The obtained target-specific Lassos are simultaneously hybridized to a collection of DNA primers that are covalently tethered via their 5′-end to labeled beads. Each primer is designed to be complementary either to the Lasso antisense sequence or to a “Zip-code” sequence, which is embedded in an internal loop of the Lasso and assigned to a particular target. Each primer sequence is also associated with a unique color-coded bead.

(6) The hybridized primers are extended by RT over the invariant 5′-end Lasso sequence. This extension will add a universal tag (u-tag) sequence to the primers attached to the beads. Since the complexity of the template sequences in Steps 5 and 6 is dramatically reduced in comparison to the initial total RNA, the specificity of RT is expected to be very high.

(7) RNA Lassos are removed from the resulting RNA-DNA hybrid by degradation of the RNA (e.g., under mild alkali conditions or by RNase H) so that the u-tag can be hybridized with oligodeoxynucleotides carrying appropriate signal amplification elements (e.g. bDNA). Since the sequence of the signal oligonucleotides will be target-independent, the hybridization conditions can be easily optimized for fast and specific binding.

(8) Both signal quantification, which is proportional to the amount of target-specific Lassos captured on the bead surface, and bead assignment can be performed using a specialized flow cytometer.

Steps 5 to 8 are schematically shown in FIG. 9B.

The extension products can be detected either by sandwich hybridization assays using either standard ELISA or bDNA/DNA dendrimer techniques (FIGS. 9B and 9D, panel a).

Alternatively, rolling circle polymerization can provide signal amplification without need to wash or destroy the Lasso probes bound to the tethered primers (FIGS. 9C and 9D, panel b).

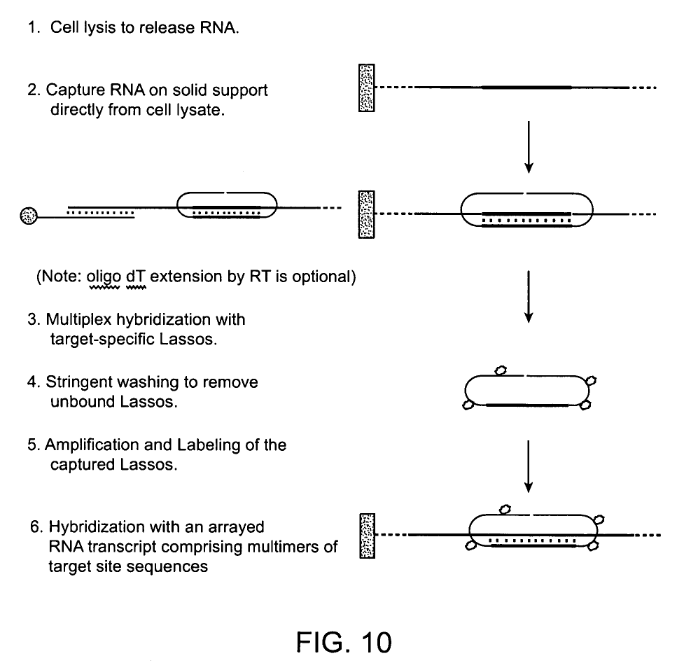

In an alternative scheme to the LAMP scheme, the RNA targets are captured on the surface of magnetic beads directly from cell/tissue lysates. Target capture can be done either using chemical/UV cross-linking or by hybridization with target-specific oligonucleotides attached to the magnetic beads at their 5′ ends. Such oligonucleotides should provide (either through chemical modification, or extended length, or primer extension) high affinity to the target to survive stringent hybridization with Lasso or other amplifiable polynucleotide probes. The probes, which are captured at magnetic beads through hybridization with the immobilized targets, are recovered and enzymatically amplified (e.g., by RT-PCR and/or transcription). The amplified probes are labeled either during transcription or by direct chemical modification. The labeled probes then are detected by hybridization with second polynucleotide probes comprising synthetic monomers or multimers of the target sequences which are arrayed on a solid support (such as color-coded beads, or 2D slide arrays, or 3D gel arrays, or dot/slot blots, or micro capillary). Alternatively, the amplified and labeled target-specific probes can be detected by mobility shift assays by gel- or capillary electrophoresis. (FIG. 10).

### Example 9

### Preparation of Lasso RNA Oligonucleotide Probes

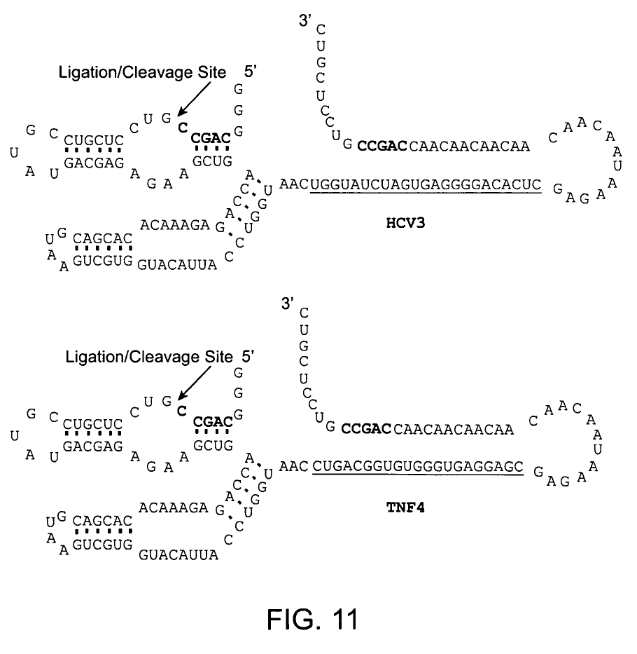

Lassos to detect TNF RNA (TNF4) and HCV RNA (HCV3) were designed and prepared. Each Lasso contains a 21-23 nt antisense sequence in addition to sequences encoding hairpin ribozymes and linker sequences. For each Lasso, four overlapping DNA oligonucleotides were used. FIG. 11 shows the sequence and proposed secondary structure of each Lasso. The antisense sequence for TNF4 Lasso probe corresponds to the coding region of the mRNA (CUGACGGUGUGGGUGAGGAGC) while the antisense sequence for HCV 3 Lasso probe targets the IRES element of the HCV RNA (UGGUAUCUAGUGAGGGGACACUC).

To prepare the in vitro transcription template, two overlapping oligonucleotides were annealed and overhangs were filled in by Klenow extension. The oligonucleotides were annealed at 80° C. for 5 minutes and slowly cooled to room temperature over the course of an hour. Two additional primers were used to amplify this sequence using PCR and to add a T7 promoter sequence.

PCR products were purified and used as templates for in vitro run off transcription by T7 RNA Polymerase. Lassos were in vitro transcribed using T7 RNA polymerase (Promega) for 3-5 hours at 37° C. using [³²P-α]CTP in the transcription mixture. Transcripts were desalted over a G50 micro-spin column (Amersham) and were stored at −20° C. until further use. RNA Lasso probes sequences:

HCV3:
GGGCAGCCGUCCUCGUCCGUAUGACGAGAGAAGCUGACCAGAGAAACACA
CGACGUAAGUCGUGGUACAUUACCUGGUAACUGGUAUCUAGUGAGGGGAC
ACUCGAGAAUAACAACAACAACAACAACCAGCCGUCCUCGUC
TNF4:
GGGCAGCCGUCCUCGUCCGUAUGACGAGAGAAGCUGACCAGAGAAACACA
CGACGUAAGUCGUGGUACAUUACCUGGUAACCUGACGGUGUGGGUGAGGA
GCGAGAAUAACAACAACAACAACAACCAGCCGUCCUCGUC

### Example 10

### Hybridization Assays for Sensitivity

Preparation of Positively Charged Membranes with Immobilized Target RNAs (Dot Blot Format)

In this assay, decreasing amounts of TNF target RNA (typically 500 ng to 0.5 ng) were mixed with a fixed amount of total cellular RNA (250 ng -1 μg, depending on experiment), which was extracted either from human embryonic carcinoma cells NTERA-2cl.D1 or 293FT cells. The RNA mixtures were spotted on a positively charged nylon membrane (Immobilon-Ny+, Millipore) using a dot blot apparatus (Minifold 1, Schleicher & Schuell). The spotted RNA molecules were immobilized to the membrane by UV-cross-linking using a standard dot-blot procedure (120 milliJoule dose, UV Stratalinker 2400, Stratagene). An excess of internally ³²P-labeled TNF probe was incubated in Church buffer (0.5M sodium phosphate pH 7.2, 1 mM sodium EDTA, and 7% sodium dodecyl sulfate) with the membrane-cross-linked target RNAs overnight at 37° C. in a hybridization incubator (Robbins Scientific). A series of washes of increasing stringency to remove unhybridized probes from the membrane was performed as shown in Table 2.

TABLE 2
Wash stringency

| Wash step | Buffer | Temperature |
|---|---|---|
| 1 | 2X SSC, 1% SDS | 37° C. |
| 2 | 0.1X SSC, 0.1% SDS, 1 mM EDTA | 37° C. |
| 3 | 0.1X SSC, 0.1% SDS, 1 mM EDTA | 50° C. |
| 4 | 0.1X SSC, 0.1% SDS, 1 mM EDTA | 65° C. |

To track the progress of the wash steps, the membrane was removed from the hybridization tube after wash steps 3 and 4, imaged using a storage phosphor screen (Molecular Imager FX, Bio-Rad), and then replaced in the hybridization tube for the next wash step. Signal-to-noise ratios were calculated based on the background signal of the mock sample (S/N=1).

### Example 11

### Hybridization Assay for SNP-Specificity (Dot Blot Format)

Positively charged membranes were prepared as in Example 10 but were spotted with equal amounts of target RNAs containing from 1-3 mismatches in the target region to Lasso TNF4 in addition to a perfectly matched target RNA in discrete spots. Target RNAs containing mismatches to the TNF4 Lasso probe were prepared by in vitro transcription of templates that were modified by site-directed mutagenesis of a plasmid encoding TNF (natural sequence) (FIG. 7c). RNA hybridization assays, wash steps, and data analysis were the same as in Example 2.

### Example 12

### RNA Lasso Probes Detect Target RNA with Greater Sensitivity than Both Long and Short DNA Probes

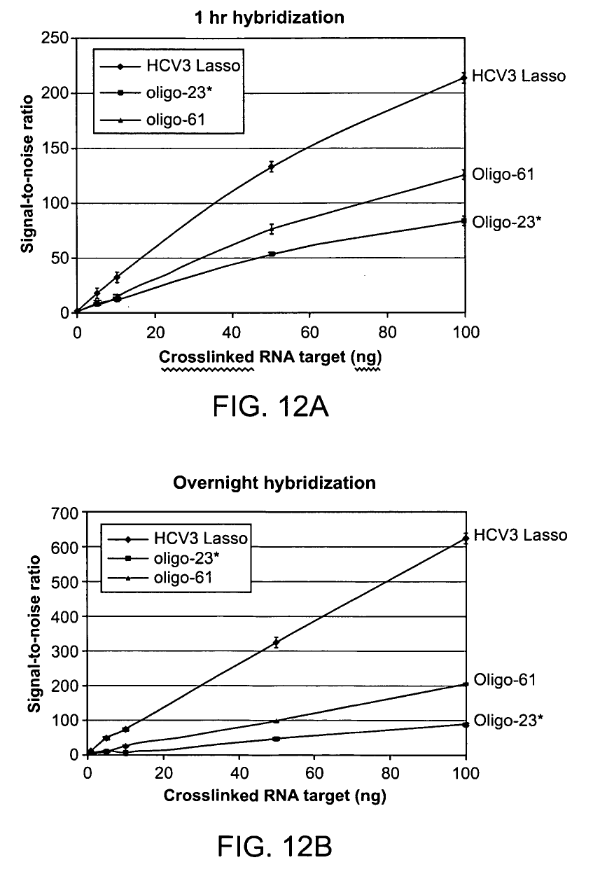

Hybridization of internally ³²P-labeled HCV3 RNA Lasso was compared with two different ³²P-5′-end-labeled DNA probes. HCV-Oligo-23 DNA probe is 23 nt long (CTCACAGGGGAGTGATCTATGGT) and comprises the same 23 nt antisense sequence as the Lasso probe. HCV-oligo-61 comprises 61 nt of HCV IRES antisense sequence that contains the 23 nt sequence targeted by the Lasso and is extended 48 additional nucleotides (GCGTGAAGACAGTAGTTCCTCACAGGGGAGTGATCTATGGTGGAGTGTCGCCCCCAATC GG). Sensitivity assays were performed as described in Example 10 with target amounts spotted ranging from 1 ng to 100 ng. Hybridizations were carried out for 1 hr or 16 hrs at 37° C. Washes were carried out as described above. FIG. 12 shows the calculated signal/noise ratios for each probe for each hybridization condition. In both shorter and longer hybridization assays, the signal/noise sensitivity of the Lasso probe is superior to the DNA probes.

### Example 13

### Design and Preparation of Lasso Probes Containing an Internal Stem-Loop Stringency Element

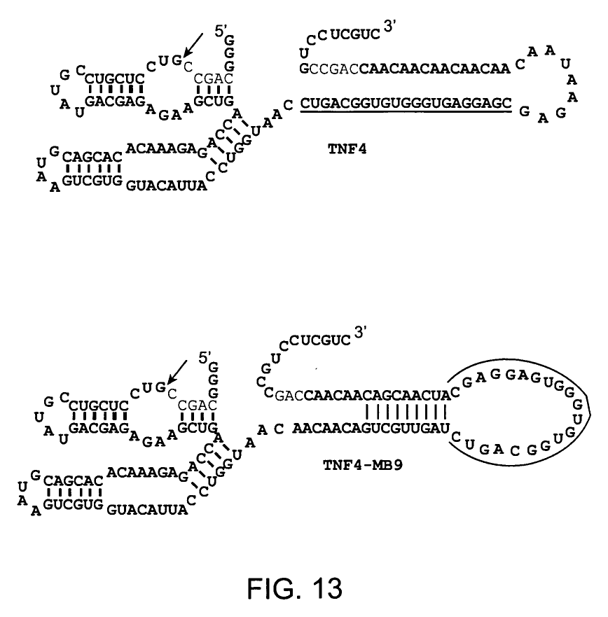

RNA anti-TNF Lassos TNF4-DB and TNF4-MB9 were prepared by in vitro transcription by T7 RNA polymerase from appropriate DNA templates. These Lassos contain internal base pairs that increase the stringency of hybridization. FIG. 13 shows sequence and secondary structure of these Lassos in comparison to TNF4 “parent” Lasso. Sequences of the probes are listed below (antisense to target RNA in bold, internal stringency elements underlined).

TNF4-DB:
GGGCAGCCGUCCUCGUCCGUAUGACGAGAGAAGCUGACCAGAGAAACACA
CGACGUAAGUCGUGGUACAUUACCUGGUAACCUGACGGUGUGGGUGAGGA
GCGAGAAUAACAACCUCCUCACAACAACAACAACAACCAGCCGUCCUCGU
C
TNF4-MB9:
GGGCAGCCGUCCUCGUCCGUAUGACGAGAGAAGCUGACCAGAGAAACACA
CGACGUAAGUCGUGGUACAUUACCUGGUAACAACAGUCGUUGAUCUGACG
GUGUGGGUGAGGAGCAUCAACGACAACAACCAGCCGUCCUCGUC

### Example 14

### RNA Lassos are More Sensitive and More Sequence Specific than DNA Probes for RNA Targets

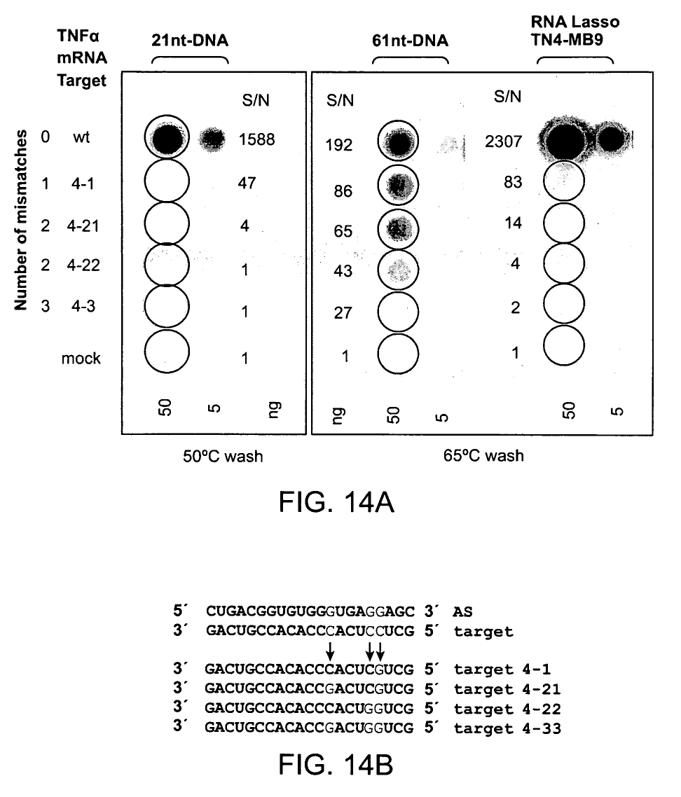

A SNP-specificity assay was performed as described in Example 11 comparing 3 probes: RNA Lasso TNF4-MB9, TNF-DNAoligo 21, and TNF DNAoligo-61. TNF-DNAoligo 21 (CTGACGGTGTGGGTGAGGAGC) has the same antisense sequence as contained in all TNF4-derived Lasso except it contains deoxynucleotides. TNF DNAoligo-61 is a deoxyoligonucleotide complementary to 61 nt of TNF mRNA also containing the 21 nucleotides of the previously described DNA probe and TNF4 Lassos (GAGATAGCAAATCGGCTGACGGTGTGGGTGAGGAGCACGTAGTCGGGGCAGCCTTGTCC CT). FIG. 14 shows the results of the specificity assay. First, although TNF-DNAoligo 21 was capable of SNP discrimination, it was completely washed off the membrane after increasing the temperature above 50° C. TNF DNAoligo-61 showed only a 2-fold reduction in signal when compared to target containing 1 mismatch vs perfectly matched target. Overall RNA Lasso TNF4-MB9 had highest sensitivity (S/M ratio for perfectly matched targets) and specificity (28-fold reduction in signal when comparing 1 mismatch to perfectly matched targets.

### Example 15

### RNA Lassos are More Sensitive and More Sequence-Specific than DNA Probes for DNA Targets

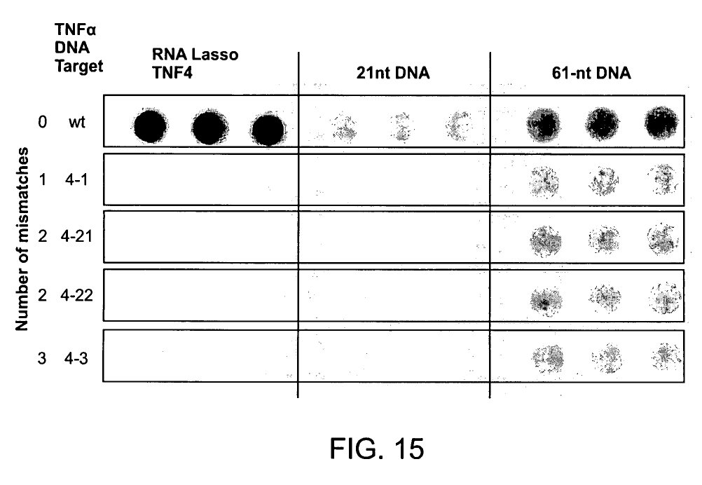

TNF dsDNA was denatured and immobilized on a positively charged nylon membrane by uv cross-linking. Hybridization conditions were the same as for RNA-spotted membranes. Sensitivity of RNA Lasso TNF4 was compared with TNF-DNAoligo 21 and TNF DNAoligo-61 and is shown in FIG. 15.

### Example 16

### RNA Lassos are More Sensitive than Short RNA Probes for RNA Targets

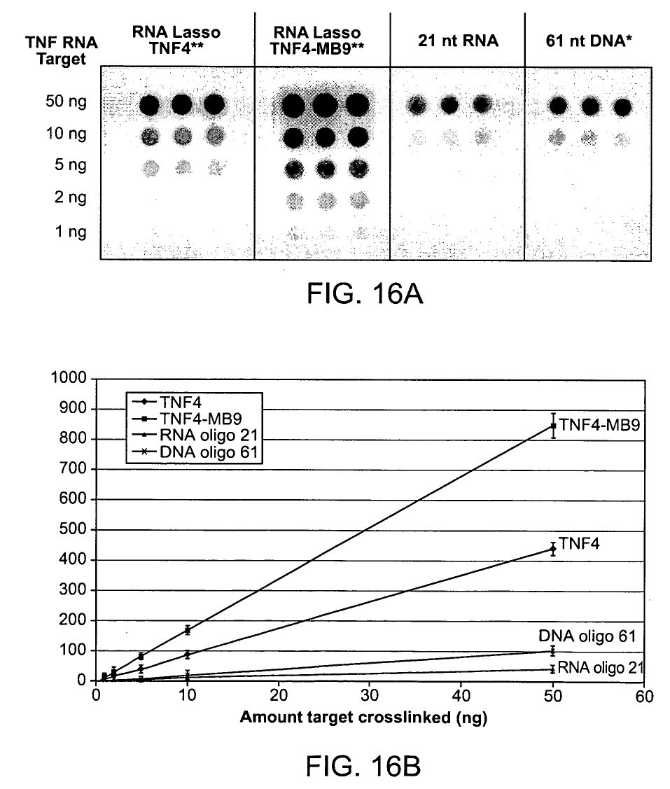

FIG. 16 shows the results of a sensitivity assay comparing the hybridization efficacy of two RNA Lasso probes (TNF4, TNF4-MB9) with TNF-DNAoligo 21, TNF DNAoligo-61 and a short 21 nt RNA probe TNF RNAoligo-21 that contains the same short antisense sequences as the Lassos (CUGACGGUGUGGGUGAGGAGC). S/N was compared after the most stringent wash of 0.1×SSC, 0.1% SDS, 1 mM EDTA at 65° C. Under these conditions TNF-DNAoligo 21 is completely washed out and so results are not shown. Both TNF4 and TNF4-MB9 were more sensitive than both the short RNA probe and long DNA probe.

### Example 17

### RNA Lassos are More Sensitive to Mismatches than Shorter RNA Probes for RNA Targets

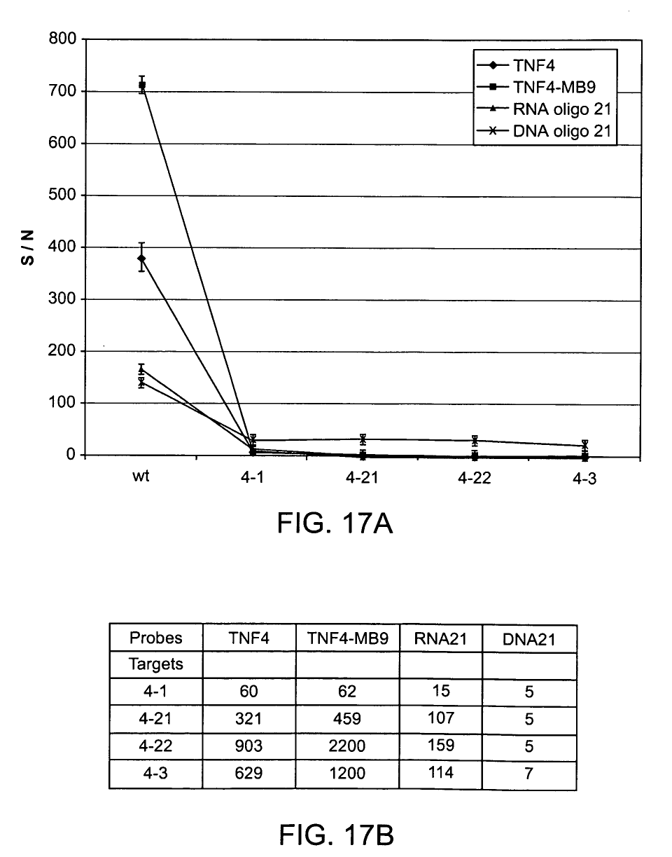

An SNP-specificity assay (Example 11) was performed comparing the same probes as in Example 9. After hybridization and stringent washing, the Lasso probes are shown to be more sensitive to mismatches than all other probes including the short 21 nt RNA probe TNF RNAoligo-21 (FIG. 17).

### Example 18

### Preparation of Genotype-Specific Lassos for HCV RNA and Genotype-Specific HCV IRES RNA

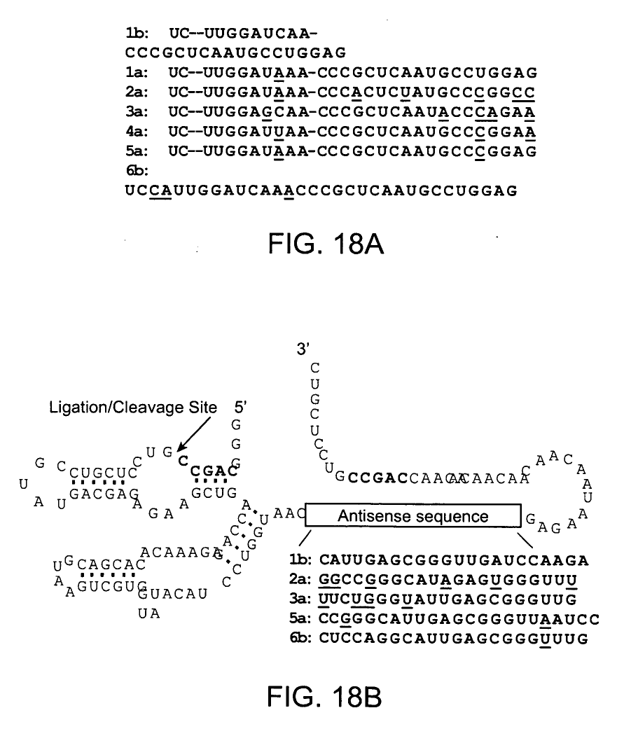

Site-directed mutagenesis (Quick-Change method, Stratagene) was used to introduce mutations into a plasmid encoding HCV IRES for genotype 1b. The mutations were clustered from nt 195-224 to make plasmids encoding for HCV IRES corresponding to genotypes 1a, 2a, 3a, 4a, 5a, and 6b in this region of the IRES (FIG. 18A). Mutations were confirmed by sequencing the resulting plasmids. Genotype-specific HCV IRES RNAS (from 195-224) were synthesized by in vitro transcription by T7 polymerase from PCR-generated transcription templates that were amplified from the mutated plasmids. Lassos were prepared containing unique antisense sequences complementary to 5 common genotypes of HCV. FIG. 18B shows the sequences secondary structures of each genotype-specific Lasso. Transcription templates were prepared and transcribed as described in Example 1. The RNA sequences of the Lassos are as follows (antisense underlined):

HCVgt1b:
GGGCAGCCGUCCUCGUCCGUAUGACGAGAGAAGCUGACCAGAGAAACACA
CGACGUAAGUCGUGGUACAUUACCUGGUAACCAUUGAGCGGGUUUAUCCA
AGAGAGAAUAACAACAACAACAACAACCAGCCGUCCUCGUC
HCVgt2a:
GGGCAGCCGUCCUCGUCCGUAUGACGAGAGAAGCUGACCAGAGAAACACA
CGACGUAAGUCGUGGUACAUUACCUGGUAACGGCCGGGCAUAGAGUGGGU
UUGAGAAUAACAACAACAACAACAACCAGCCGUCCUCGUC
HCVgt3a:
GGGCAGCCGUCCUCGUCCGUAUGACGAGAGAAGCUGACCAGAGAAACACA
CGACGUAAGUCGUGGUACAUUACCUGGUAACUUCUGGGUAUUGAGCGGGU
UGGAGAAUAACAACAACAACAACAACCAGCCGUCCUCGUC
HCVgt5a:
GGGCAGCCGUCCUCGUCCGUAUGACGAGAGAAGCUGACCAGAGAAACACA
CGACGUAAGUCGUGGUACAUUACCUGGUAACCCGGGCAUUGAGCGGGUUA
AUCCGAGAAUAACAACAACAACAACAACCAGCCGUCCUCG
UC
HCVgt6b:
GGGCAGCCGUCCUCGUCCGUAUGACGAGAGAAGCUGACCAGAGAAACACA
CGACGUAAGUCGUGGUACAUUACCUGGUAACCUCCAGGCAUUGAGCGGGU
UUGGAGAAUAACAACAACAACAACAACCAGCCGUCCUCGU
C

### Example 19

### Lasso Hybridization Probes are Capable of Genotype-Specific Discrimination of HCV IRES

A dot blot “array” was prepared spotting each of the genotype-specific RNAs on a positively charge nylon membrane and immobilizing by uv cross-linking with a 120 mJ dose. A hybridization assay was performed where each one of the genotype specific Lassos was incubated individually over 16 hours in Church buffer at 37° C. with the spotted membrane containing each genotype specific HCV IRES target cross-linked to it. A series of increasingly stringent washes was performed as follows (Table 3):

TABLE 3
Was stringency

| Wash step | Buffer | Temperature |
|---|---|---|
| 1 | 2X SSC, 1% SDS | 37° C. |
| 2 | 0.1X SSC, 0.1% SDS, 1 mM EDTA | 37° C. |
| 3 | 0.1X SSC, 0.1% SDS, 1 mM EDTA | 50° C. |
| 4 | 0.1X SSC, 0.1% SDS, 1 mM EDTA | 60° C. |

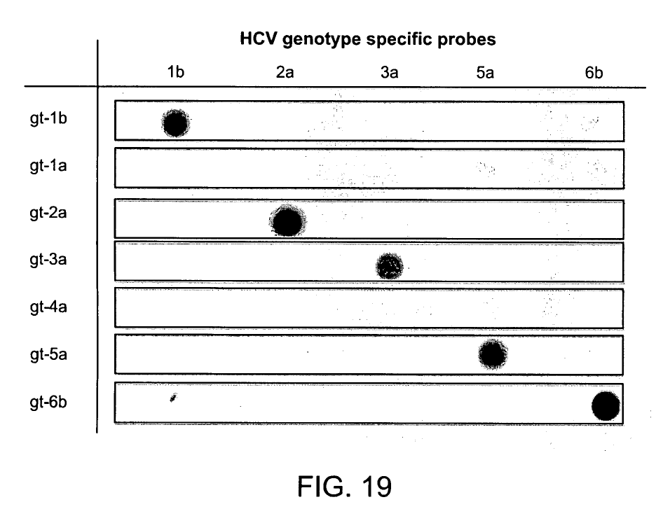

FIG. 19 shows the result of the hybridization experiment in which each of the 5 genotype specific Lassos hybridizes preferentially to its cognate genotype target RNA.

### Example 20

### Lasso Probes with Deleted Linker Sequences and Inverted Hairpin Ribozyme Sequences are Capable Highly Sensitive Detection of TNF Target RNA

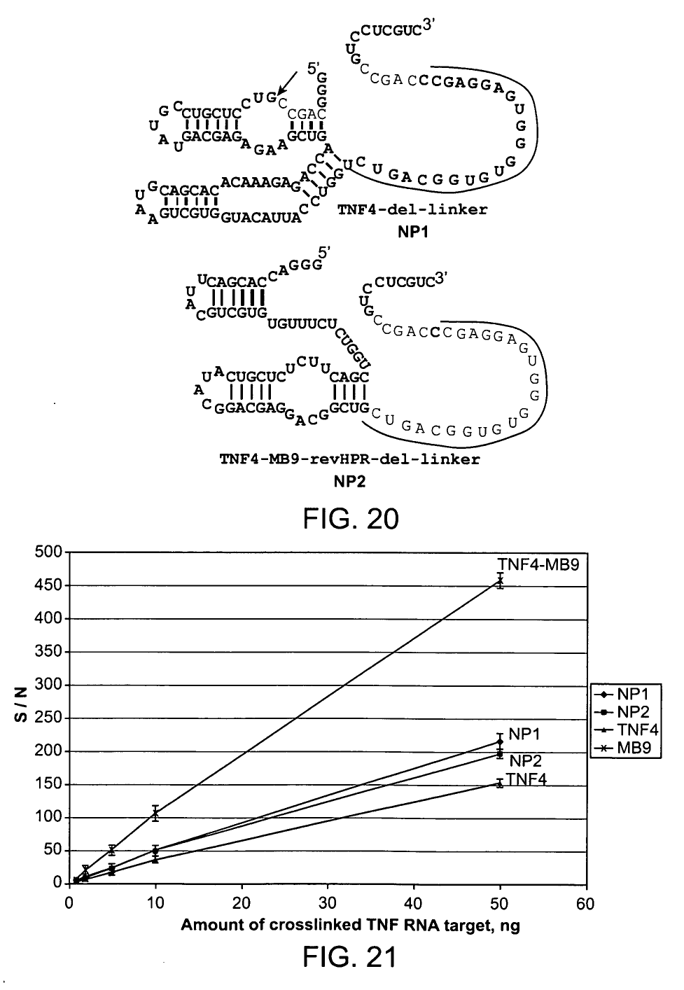

A Lasso probe (NP1) with omitted linker sequences between the hairpin ribozyme and antisense sequence (to TNF mRNA) and between the antisense sequence and the 3′ cleavage/ligation site was prepared by in vitro transcription by T7 RNA polymerase using a double stranded DNA template prepared from corresponding DNA oligonucleotides (FIG. 20A). In addition, a probe (NP2) in which the sequence of the hairpin ribozyme was inverted such that the secondary structure was preserved but the enzymatic activity of the ribozyme was abolished was prepared in similar fashion (FIG. 20B).

NP1 sequence:
GGGCAGCCGUCCUCGUCCGUAUGACGAGAGAAGCUGACCAGAGAAACACA
CGACGUAAGUCGUGGUACAUUACCUGGUCUGACGGUGUGGGUGAGGAGCA
CCAGCCGUCCUGCUC
NP2 sequence:
GGGACCACGACTTACGTCGTGTGTTTCTCTGGTCAGCTTCTCTCGTCATA
CGGACGAGGACGGCTGCUGACGGUGUGGGUGAGGAGCACCAGCCGUCCUG
CUC

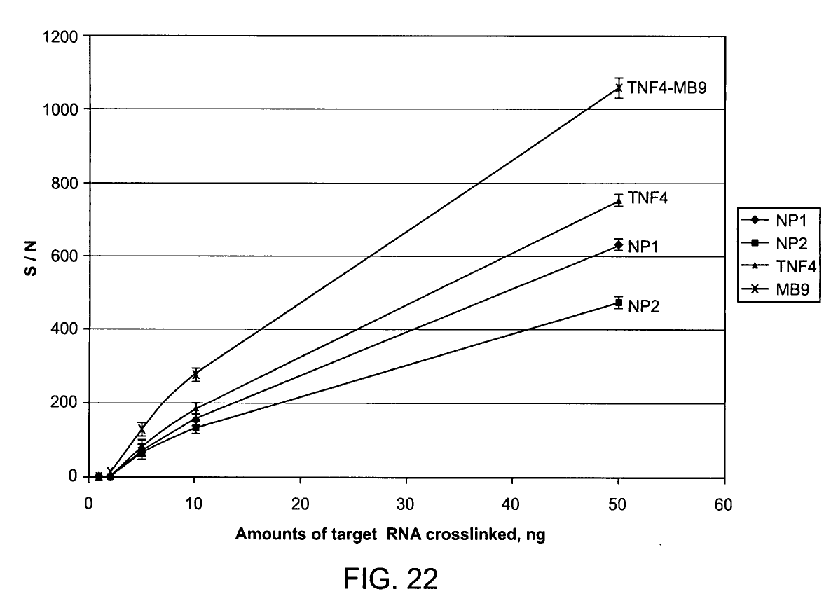

These probes were tested in a sensitivity assay (as described in Example 10) in parallel with Lasso probes TNF4 and TNF4-MB9. Assays were carried out both with total RNA (FIG. 21) and without total RNA (FIG. 22) included in each dot on the blot. While TNF4-MB9 had the highest sensitivity in both assays, probes NP1 and NP2 also exhibited high signal/background values of mRNA detection both in the presence and absence of total RNA.

### Example 21

### Deletion of Hairpin Ribozyme-Like Sequences from RNA Probes Results in Probes that are not as Sensitive as Lasso Probes Containing these Sequences

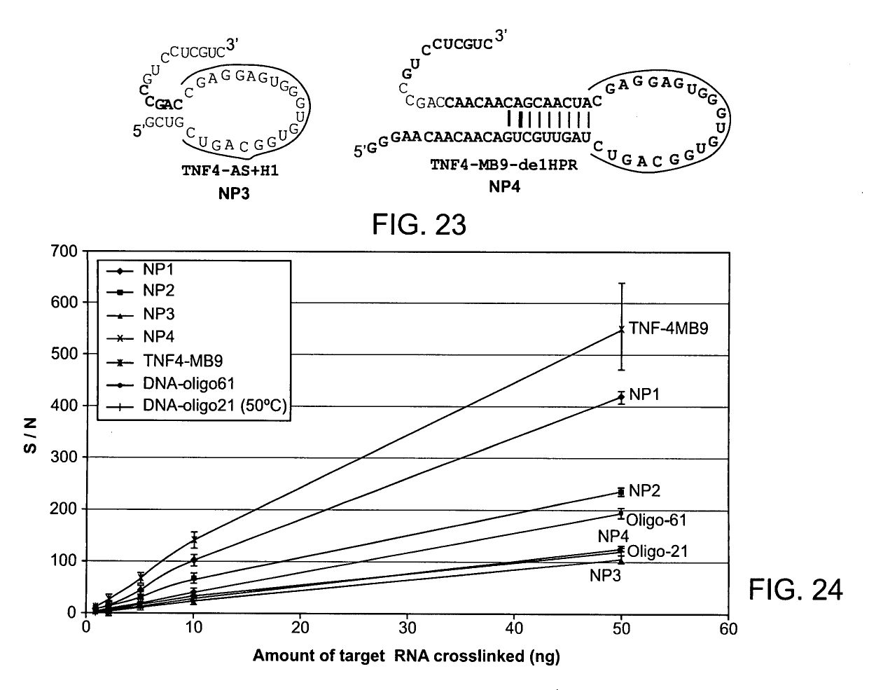

Two RNA probes were prepared with the following sequences and secondary structures (FIG. 23):

NP3:
GCUGAUCUGACGGUGUGGGUGAGGAGCCAGCC
NP4:
GGGAACAACAACAGUCGUUGAUCUGACGGUGUGGGUGAGGAGCAUCAACG
ACAACAACCAGCCGUCCUGCUC

NP3 contains the antisense sequence to TNF RNA and a 4 bp helix. NP4 contains the 3′ section of the Lasso TNF4-MB9 that contains the internal stem-loop element and antisense sequences and lacks the hairpin ribozyme domain entirely. These probes were tested in parallel in sensitivity assays with TNF4-MB9, NP1, NP2, TNF-DNAoligo 21, and TNF-DNAoligo 61. Signal/background values were plotted after washing all probes except TNF-DNAoligo 21 at 65° C. in 0.1×SSC, 0.1% SDS, 1 mM EDTA. Signal/background for probe TNF-DNAoligo 21 was calculated after washing in the same buffer at 50° C. since the this probe is completely washed out at the higher temperature. FIG. 24 shows the results that NP3 and NP4 have lower sensitivity than the longer Lasso probes TNF4-MB9, NP1, and NP2. Overall, the signal/background values of NP3 and NP4 are lower than that of TNF-DNAoligo 61.

### Example 22

### Deletion of Linker Sequences or Hairpin Ribozyme-Like Sequences has a Varying Effect on SNP-Discrimination Capability of RNA Probes

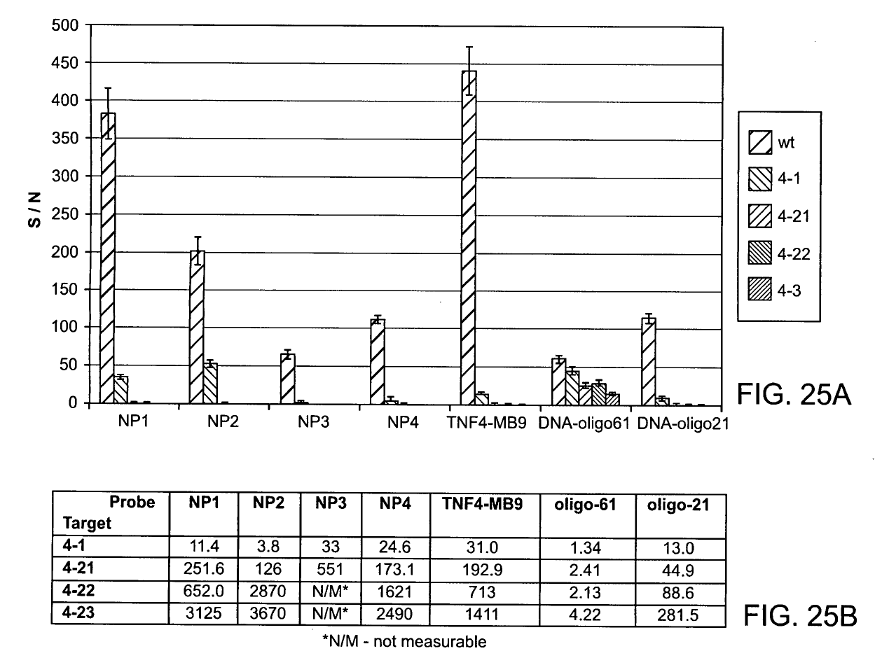

An assay that measures sensitivity of probes to mismatches (single, double, triple) in target TNF RNA was performed as described in Example 11. Probes tested were the same as in Example 14. NP1, NP2, NP3, and NP4 are all capable of discrimination of SNP albeit to varying extents. Of the four probes, NP2 had the lowest discriminatory capability, with a four-fold reduction in signal/background from perfectly matched to single mismatched targets (FIG. 25). While NP3 and NP4 were highly sensitive to SNP-discrimination (25-33 fold difference), their overall sensitivity of target detection was much lower than both NP1 and TNF4-MB9.

### Example 23

### Non-Circularizing Lasso-Like Probes are Capable of Sensitive Detection of RNA Targets with High Signal-to-Noise

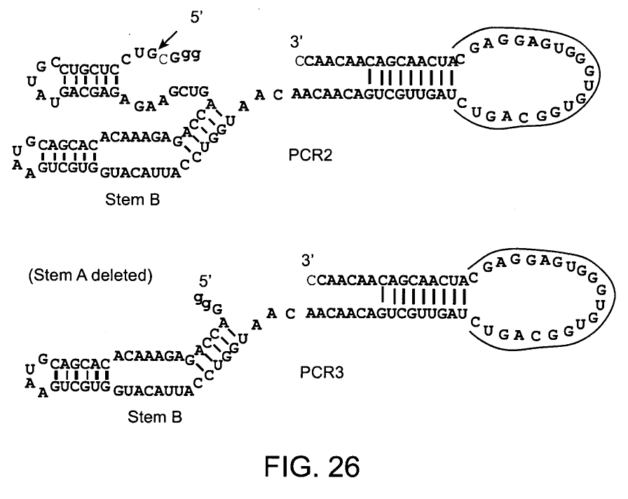

Sensitivity assays were performed with and without total RNA comparing signal/background levels for Lasso probes that have 5′ and 3′ end deleted sequences. FIG. 26 shows the sequences and secondary structures of probes PCR2 and PCR3.

PCR2 sequence:
GGGCGUCCUCGUCCGUAUGACGAGAGAAGCUGACCAGAGAAACACACGAC
GUAAGUCGUGGUACAUUACCUGGUAACAACAGUCGUUGAUCUGACGGUGU
GGGUGAGGAGCAUCAACGACAACAACC
PCR3 sequence:
GGGACCAGAGAAACACACGACGUAAGUCGUGGUACAUUACCUGGUAACAA
CAGUCGUUGAUCUGACGGUGUGGGUGAGGAGCAUCAACGACAACAACC

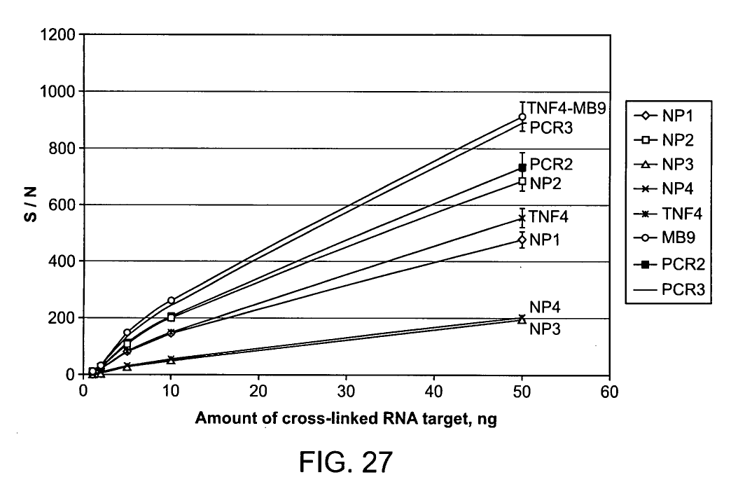

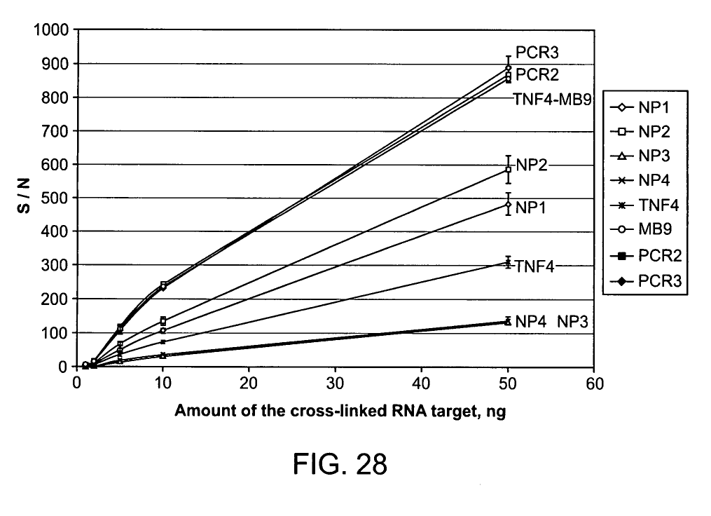

Probe PCR2 has deleted sequences at the 5′ and 3′ ends of the Lasso RNA corresponding to sequences that are necessary for docking into the active site of the hairpin ribozyme. PCR3 probe has the entire stem A of the hairpin ribozyme deleted. Neither of these probes is capable of circularization around target RNA. The sensitivity assay was carried out in parallel with previously characterized probes NP1, NP2, NP3, NP4, TNF4, and TNF4-MB9. FIGS. 27 and 28 show that removal of sequences at the 5′ and 3′ end of the Lasso probe as shown in FIG. 26 do not affect the sensitivity of the RNA probes appreciably.

### Example 24

### Preparation of RNA Lasso-Like Probes with Deletions of Stem B of Hairpin Ribozyme Sequences

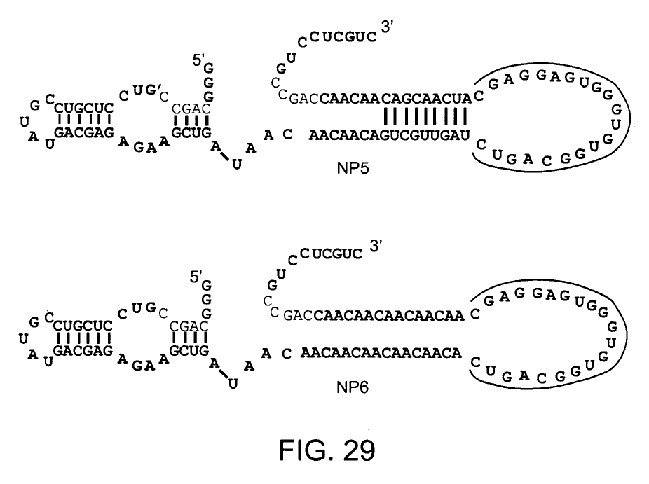

The following Lasso-like probes were prepared by in vitro transcription of appropriate DNA templates. FIG. 29 shows the sequences and secondary structures of NP5 and NP6. NP5 (shown below) is derived from TNF4-MB9 but sequences encoding stem B of the hairpin ribozyme have been removed:

GGGCAGCCGUCCUCGUCCGUAUGACGAGAGAAGCUGAUAACAACAACAGU
CGUUGAUCUGACGGUGUGGGUGAGGAGCAUCAACGACAACAACCAGCCGU
CCUCGU

NP6 (shown below) is similar to NP5 in length and omission of stem B but sequences with encoding for the internal stem loop have been replaced with nucleotides that do not form Watson-Crick pairings:

GGGCAGCCGUCCUCGUCCGUAUGACGAGAGAAGCUGAUAACAACAACAAC
AACAACACUGACGGUGUGGGUGAGGAGCAACAACAACAACAACCAGCCGU
CCUCGUC.

### Example 25

### Design of All RNA Double Stem Loop (DSL) Probe

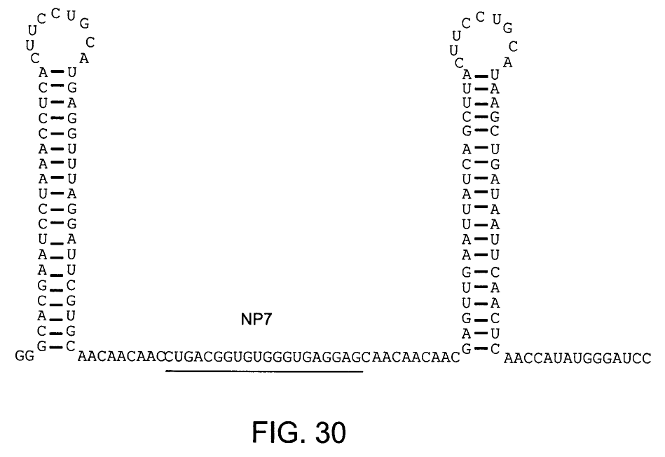

To test the effect of combining a short antisense sequence as with RNA Lassos with sequences that form hairpins both upstream and downstream of the antisense sequence, the following RNA probe (NP7) to detect TNF RNA was designed and prepared (FIG. 30):

GGGCACGAAUCCUAAACCUCACUUCCUGCAUGAGGUUUAGGAUUCGUGCA
ACAACAACCUGACGGUGUGGGUGAGGAGCAACAACAACGAGUUGAAUUAU
CAGCUUACUUCCUGCAUAAGCUGAUAAUUCAACUCAACCAUAUGGGAUCC

Both hairpins at the 5′ and 3′ sides of the antisense sequence are not complementary to the target RNA (TNF RNA).

### Example 26

### Probes with Stem B Deletions of the Hairpin Ribozyme and a Novel DSL RNA Probe are Capable of Detecting RNA Targets with High Signal-to-Noise

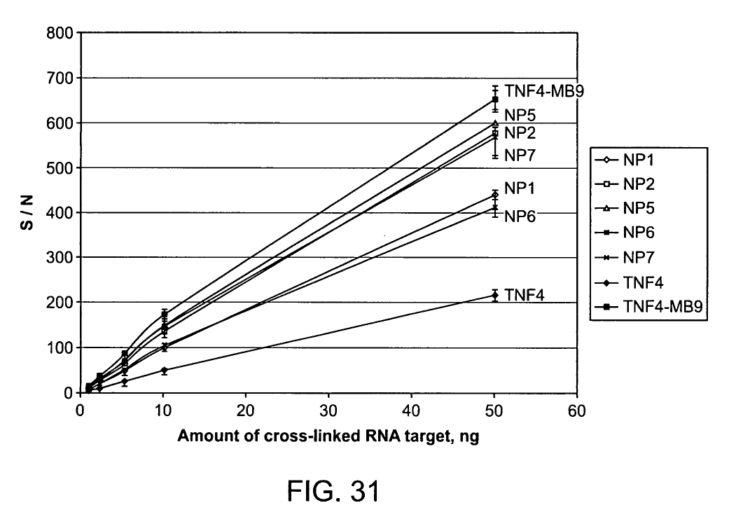

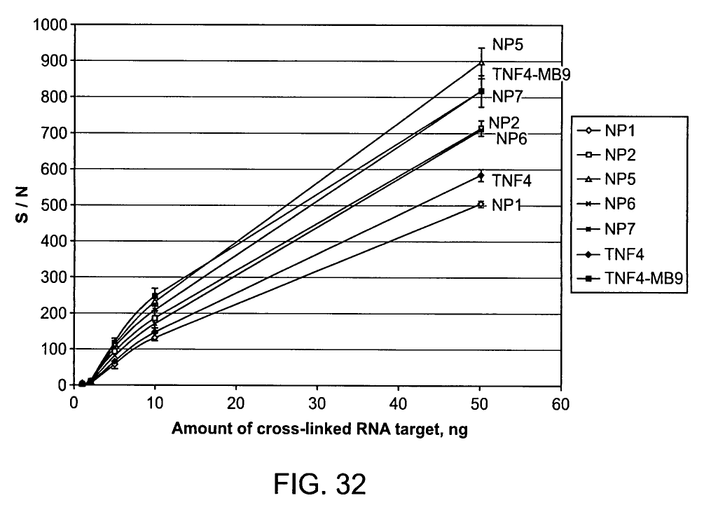

FIGS. 31 and 32 show the results of sensitivity assays both with and without total RNA included (as described in Example 10) for the probes NP1, NP2, NP5, NP6, NP7 (DSL), TNF4, and TNF4-MB9. Both NP5 (stem B deletion, with internal stem loop) and NP7 (DSL probe) were capable of detecting RNA with comparable sensitivity to full-length RNA Lasso TNF4-MB9.

### Example 27

### Effect on SNP Sensitivity from Modification of Lasso Probe Sequences

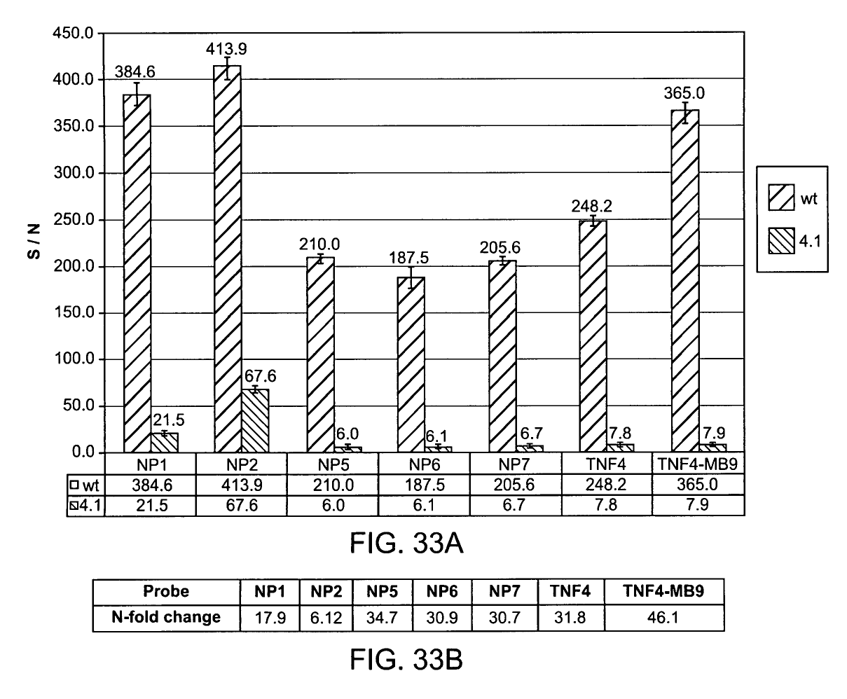

A SNP-discrimination assay was performed as described in Example 11 for the same set of probes used in Example 19. As shown in FIG. 33, NP5, NP6, and NP7 showed an SNP-discrimination capability similar to full length RNA Lassos.

### REFERENCES

Allawi, H. T., et al. 2001. RNA 7, 314-327.
Antson, D. O., et al. 2000. Nucleic Acids Res. 28: e58.
Baner, J., et al. 2001. Curr. Opin. Biotechnol. 12: 11-15.
Beaudry, D., Perreault, J.-P. 1995. Nucleic Acids Res. 23: 3064-3066.
Berzal-Herranz, A., Burke, J. M. 1997. Methods Mol. Biol. 74: 349-355.
Bisucci, T., et al. 2000. Methods Mol. Biol. 123: 291-303.
Bonnet, G., et al. 1999. Proc. Natl. Acad. Sci. USA 96: 6171-6176.
Braasch, D. A., et al. 2002. Nucleic Acids Res. 30: 5160-7.
Brantl, S. 2002. Plasmid 48: 165-73.
Breier, G. 1999. Methods Mol. Biol. 96, 107-17.
Broude, N. E., et al. 2001a. Proc. Natl. Acad. Sci. USA 98: 206-11.
Broude, N. E., et al. 2001b. Antisense Nucleic Acid Drug Dev. 11: 327-32.
Brownie, J., et al. 1997. Nucleic Acids Res. 25: 3235-41.
Bruice, T. W., Lima, W. F . 1997. Biochemistry 36: 5004-5019.
Brukner, I., et al. 2002. Biotechniques 33, 874-6, 878, 880.
Certa, U., et al. 2001. Methods Mol. Biol. 170: 141-156.
Chen, C., et al. 2005. Nucleic Acids Res. 33: e179.
Chou, C. C., et al. 2004. Nucleic Acids Res. 32: e99.
Christian, A. T., et al. 2001. Proc. Natl. Acad. Sci. USA 98: 14238-14243.
Church, G. M., Gilbert, W. 1984. Proc. Natl. Acad. Sci. USA 81: 1991-1995.
Collins, M. L., et al. 1997. Nucleic Acids Res. 25: 2979-84.
Delihas, N., et al. 1997. Nat. Biotechnol. 15: 751-3.
Desombere, I., et al. 2005. J. Clin. Microbiol. 43: 2590-2597.
Ding, C., Cantor, C. R. 2004. J. Biochem. Mol. Biol. 37: 1-10.
Dunbar, S. A. 2006. Clin. Chim. Acta 363: 71-82.
Ekins R., Chu, F. W. 1999. Trends Biotechnol. 17: 217-218.
Fan, J. B., et al. 2000. Genome Res. 10: 853-860.
Favis, R., et al. 2000. Nat. Biotechnol. 18: 561-564.
Fedor, M. J. 2000. J. Mol. Biol. 297: 269-291.
Feldstein, P. A., Bruening, G. 1993. Nucleic Acids Res. 21: 1991-1998.
Flagella, M., et al. 2006. Anal Biochem. 352: 50-60.
Flaspohler, J. A., Milcarek, C. 1992. BioTechniques 13: 68-72.
Fluiter, K., et al. 2005. Oligonucleotides 15: 246-54.
Fonseca, M. E., et al. 1996. J. Virol. Methods 57: 203-7.
Gerry, N. P., et al. 1999. J. Mol. Biol. 292: 251-62.
Gryaznov, S., Lloyd, D. H. 1995. U.S. Pat. No. 5,817,795.
Guo, Z., et al. 1997. Nat. Biotechnol. 15: 331-5.
Hacia, J. G. 1999. Nat. Genet. 21 (1 Suppl): 42-47.
Harada, K., Orgel, L. E. 1993. Nucleic Acids Res. 21: 2287-229.
Hardenbol, P., et al. 2003. Nat. Biotechnol. 21: 673-638.
Hertel, K. J., et al. 1998. Biochemistry 37: 16983-16988.
Hirschhorn, J. N., et al. 2000. Proc. Natl Acad. Sci. USA 97: 12164-12169.
Ho, S. P., et al. 1998. Nat. Biotechnol. 16: 59-63.
Homann, M., et al. 1996. Nucleic Acids Res. 24: 4395-4400.
Hougaard, D. M., et al. 1997. Histochem. Cell. Biol. 108: 335-344.
Huang, C. Y., et al. 1998. Genet. Anal. 14: 109-112.
Hughes, T. R., et al. 2001. Nat. Biotechnol. 19: 342-347.
Iannone, M. A., et al. 2000. Cytometry 39: 131-40.
Jacobsen, N., et al. 2002. Nucleic Acids Res. 30: e100.
James, K., et al. 1998. Nucleic Acids Res. 26: 5203-5211.
Johnston, B. H., et al. 1998. International Patent Application (PCT) WO99/09045A1.
Kampke, T., et al. 2001. Bioinformatics 17: 214-25.
Kandimalla, E. R., et al. 1995. Nucleic Acids Res. 23: 3578-84.
Kataja, K. et al. 2006 J. Microbiol. Meth. 67: 102-113
Kawasaki, H., et al. 2003. Nucleic Acids Res. 31: 981-987.
Kazakov, S. A., et al. 2004. International Patent Application (PCT) No. WO2005/001063 A2, published Jan. 6, 2005 (filed Jun. 25, 2004).
Kazakov, S. A., et al. 2005. International (PCT) Patent Application WO2006007569 A2, published Jan. 19, 2006 (filed Jul. 1, 2005).
Kisich, K. O., et al. 1999. J. Immunol. 163: 2008-2016.
Komatsu, Y., 1993. J. Am. Chem. Soc. 116: 3692-3696.
Komatsu, Y., et al. 1995. J. Mol. Biol. 252: 296-304.
Kothapalli, R., et al. 2002. BMC Bioinformatics 3: 22.
Kronenwett, R., Sczakiel, G. 1997. Methods Mol. Biol. 74: 281-288.
Krupp, G. 1988 Gene 72: 75-89.
Kuhn, H., et al. 2002. Nucleic Acids Res. 30: 574-580.
Kumar, M., Carmichael, G. G. 1998. Microbiol. Mol. Biol. Rev. 62:1415-34.
Kumar, R. 1999. U.S. Pat. No. 5,912,124.
Kuo, W. P., et al. 2006. Nat. Biotechnol. 24: 832-840.
Kwiatkowski, M., et al. 1996. Nucleic Acids Res. 24: 4632-4638.
Lane, M. J. et al. 1997 Nucleic Acids Res. 25: 611-617.
Lane, M. J. et al., 1998. U.S. Pat. No. 5,770,365.
Lesnik, E. A., Freier, S. M. 1995. Biochemistry 34: 10807-15.
Li, Q., et al. 2002. Nucleic Acids Res. 30: e5.
Liang, Z., et al. 2002. International Patent Application (PCT) WO 02/24950 A2.
Lima, W. F., et al. 1997. J. Biol. Chem. 272: 626-638.
Lin, H., et al. 2006. J. Am. Chem. Soc. 128: 3268-72.
Lizardi, P. M., et al. 1998. Nat. Genet. 19: 225-232.
Lloyd, B. H., et al. 2001. Nucleic Acids Res. 29: 3664-3673.
Lockhart, D. J., et al. 1996. Nat. Biotechnol. 14: 1675-1680.
Luo, J., et al. 1996. Nucleic Acids Res. 24: 3071-78.
Maher, L. J., 3rd, Dolnick, B. J. 1988. Nucleic Acids Res. 16: 3341-58.
Majlessi, M., et al. 1998. Nucleic Acids Res. 26: 2224-2249.
Mansfield, E. S., et al. 1995. Mol. Cell. Probes 9: 145-56.
Marras, S. A., et al. 2003. Methods Mol. Biol. 212: 111-128.
Marshall, E. 2004. Science 306: 630-631.
Milner, N., et al. 1997. Nat. Biotechnol. 15: 537-541.
Mittman, M. et al. 2007. U.S. Pat. No. 7,157,564.
Monia, B. P., et al. 1992. J. Biol. Chem. 267: 19954-62.
Murray, J. B., et al. 1998. Chem. Biol. 5: 587-595.
Myer, S. E., Day, D. J. 2001. Biotechniques 30: 584-588, 590, 592-593.
Nazarenko, I. A. et al. 1997 Nucleic Acids Res. 25: 2516-2521.
Nesbitt, S. M., et al. 1999. J. Mol. Biol. 286: 1009-1024.
Nilsson, M., et al. 1994. Science 265: 2085-2088.
Nilsson, M., et al. 2000. Nat. Biotechnol. 18: 791-793.
Nilsson, M., et al. 2001. Nucleic Acids Res. 29: 578-581.
Noonberg, S. B., Hunt, A. 1997. U.S. Pat. No. 5,624,803.
Ohmichi, T., Kool, E. T. 2000. Nucleic Acids Res. 28: 776-783.
Pan, W. H., et al. 2001. RNA 7: 610-21.
Patzel, V., Sczakiel, G. 2000. Nucleic Acids Res. 28: 2462-2466.
Peplies, J., et al. 2003. Appl. Environ. Microbiol. 69: 1397-1407.
Peterson, A. W., et al. 2002. J. Am. Chem. Soc. 124: 14601-14607.
Pfeffer, S., et al. 2003. Current Protocols Mol. Biol. 26.4.1.-26.4.18.
Pinto, F. L., et al. 2006. BMC Biotechnol. 6: 31.
Player, A. N., 2001. J. Histochem. Cytochem. 49: 603-612.
Pritchard, C. E., Southern, E. M. 1997. Nucleic Acids Res. 25: 3403-7.
Qi, X., et al. 2001. Nucleic Acids Res. 29: e116.
Ramkissoon, S. H., et al. 2006. Mol. Cell. Probes 20: 1-4.
Ratilainen, T., et al. 2000. Biochemistry 39: 7781-91.
Ricceli, P. V. et al. 2001. Nucleic Acids Res. 29: 996-1004.
Rittner, K., et al. 1993. Nucleic Acids Res. 21: 1381-1387.
Roberts, R. W., Crothers, D. M. 1992. Science 258: 1463-1436.
Rogler, C. E., et al. 2004. Nucleic Acids Res. 32: e120.
Scherr, M., et al. 2001. Mol. Ther. 4: 454-460.
Sczakiel, G., Far, R. K. 2002. Curr. Opin. Mol. Ther. 4: 149-153.
Seyhan, A. A., et al. 2005. RNA 10: 837-846.
Shuber, A. P., et al. 1995. Genome Res. 5: 488-93.
Shuldiner, A. R., et al. 1990. Gene 91: 139-42.
Singh, R. P., et al. 1994. J. Virol. Methods 49: 221-233.
Smith, R. D., et al. 2001. Biotechniques 31: 776-8, 780, 782.
Soderlund, H. et al. 2008. United States Patent Application Publication US2008/0233591 A1.
Sohail, M., Southern, E. M. 2000. Adv. Drug Deliv. Rev. 44: 23-34.
Sohail, M., et al. 2001. Nucleic Acids Res. 29: 2041-2051.
Song, J. Y., et al. 2005. Nucleic Acids Res. 33: e19.
Southern, E., et al. 1999. Nat. Genet. 21: 5-9.
Steel, A. B., et al. 2000. Biophys J. 79: 975-981.
Stull, R. A., et al. 1996. Antisense Nucleic Acid Drug Dev. 6: 221-228.
Sugimoto, N., et al. 1995. Biochemistry 34: 11211-11216.
Szemes, M., et al. 2005. Nucleic Acids Res. 33: e70.
Tan, P. K., et al. 2003. Nucleic Acids Res. 31: 5676-5684.
Thomas, D. C., et al. 1999. Arch. Pathol. Lab. Med. 123: 1170-1176.
Thompson, J., Gillespie, D. 1987. Anal. Biochem. 163: 281-91.
Toulme, J. J., et al. 2001. Prog. Nucleic Acid Res. Mol. Biol. 69: 1-46.
Tsai, S. P., et al. 2003. Nucleic Acids Res. 31: e25.
Tyagi, S., Kramer, F. R. 1996. Nat. Biotechnol. 14: 303-8.
Ugozzoli, L. A. 2004. Clin. Chem. 50: 1963-1965.
Valoczi, A., et al. 2004. Nucleic Acids Res. 32: e175.
Vary, C. P. 1987. Nucleic Acids Res. 15: 6883-6897.
Vlassov, A. V., et al. 2004. Oligonucleotides 14: 210-220.
Wagner, E. G., et al. 2002. Adv. Genet. 46: 361-98.
Walter, A. E., et al. 1994. Proc. Natl. Acad. Sci. USA 91: 9218-22.
Wang, D., et al. 2003. Biotechniques 35: 300-2, 304, 306 passim.
Wrzesinski, J., et al. 2000. Nucleic Acids Res. 28: 1785-1793.
Wu, D. Y., Wallace, R. B. 1989. Gene 76: 245-254.
Wu, P., et al. 2002. Eur. J. Biochem. 269: 2821-30.
Xi, X., et al. 2003. Biotechniques 34: 914-6, 918.
Ye, F., et al. 2001. Hum. Mutat. 17: 305-16.
Zeiler, B. N., Simons, R. W. 1998. In RNA structure and function. R. W. Simons, M. Grunberg-Manago (Eds.), Cold Spring Harbor: Cold Spring Harbor Laboratory Press, pp. 437-464.
Zhang, D. Y., et al. 1998. Gene 211: 277-285.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

We claim:

1. A method of detecting the presence of a target polynucleotide in a sample, said method comprising:
a) contacting said sample with a polynucleotide probe under hybridization conditions, wherein said polynucleotide probe comprises:
i) a target binding domain that is substantially complementary to a polynucleotide sequence of said target polynucleotide; and
ii) a binding enhancer domain ranging from 30 to 10,000 nucleotides in length that cannot form a stable hybridization complex with said target polynucleotide or with said target binding domain under said hybridization conditions, wherein said polynucleotide probe has an improved hybridization characteristic for said target polynucleotide as compared to said target binding domain without said binding enhancer domain; and
b) assaying for the presence of stable hybridization complexes between said polynucleotide probe and said target polynucleotide, thereby detecting the presence of said target polynucleotide in said sample.

2. The method of claim 1, wherein said wherein said target binding domain ranges from 3 to 30 nucleotides in length.

3. The method of claim 1, wherein said binding enhancer domain comprises a predetermined secondary or tertiary structure selected from one or more of: a stem-loop structure, a pseudoknot, a bipartite nucleic acid duplex, a nucleic acid triplex and a nucleic acid tetraplex.

4. The method of claim 3, wherein said predetermined secondary or tertiary structure is selected from sequences substantially related to: a catalytically active hairpin ribozyme, a catalytically inactive hairpin ribozyme, a truncated hairpin ribozyme, a tRNA, and a region from a ribosomal RNA.

5. The method of claim 1, wherein said binding enhancer domain comprises a first subdomain located 5′ of said target binding domain and a second subdomain located 3′ of said target binding domain.

6. The method of claim 1, wherein either said target polynucleotide or said polynucleotide probe is captured or immobilized on a solid support.

7. The method of claim 6, wherein said solid support is selected from: a synthetic bead, a membrane or filter, a microarray slide, microtiter plate and microcapillary.

8. The method of claim 1, wherein said hybridization characteristic is selected from one or more of: selectivity, sensitivity, affinity and binding efficacy.

9. The method of claim 1, wherein said assaying step (b) further comprises a signal amplification step.

10. The method of claim 1, wherein said assaying step (b) comprises:
i) isolating hybridization complexes comprising said polynucleotide probe and said target polynucleotide;
ii) recovering from said hybridization complexes said polynucleotide probe;
iii) hybridizing a synthesis primer to said recovered polynucleotide probe from step (ii);
iv) placing said synthesis primer-hybridized polynucleotide probe under nucleic acid synthesis conditions to extend said synthesis primer; and
v) detecting said extended synthesis primer.

11. The method of claim 1, wherein multiple target polynucleotides are detected in said sample using multiple of said polynucleotide probes each specific for one of said multiple target polynucleotides.

12. The method of claim 11, wherein each of said multiple polynucleotide probes further comprises a unique identifier domain.

13. The method of claim 11, said method further comprising capturing polynucleotides in said sample on a solid support prior to said contacting step (a) and wherein said assaying step (b) further comprises:
i) isolating hybridization complexes comprising said multiple polynucleotide probes and their corresponding target polynucleotides;
ii) recovering from said hybridization complexes said polynucleotide probes;
iii) amplifying and labeling said recovered polynucleotide probes from step (ii);
iv) hybridizing said labeled polynucleotide probes from step (iii) with multiple second polynucleotide probes each comprising a sequence from one of said target sequences, wherein said second polynucleotide probes are arrayed on a solid support;
and
v) detecting said labeled probes from step (iv).

14. A polynucleotide probe specific for a target polynucleotide comprising:
a) a target binding domain ranging from 3 to 30 nucleotides in length that is substantially complementary to a polynucleotide sequence of said target polynucleotide; and
b) a binding enhancer domain ranging from 30 to 10,000 nucleotides in length that cannot form a stable hybridization complex with said target polynucleotide or with said target binding domain under standard hybridization conditions.

15. The polynucleotide probe of claim 14, wherein said binding enhancer domain comprises a predetermined secondary or tertiary structure selected from one or more of: a stem-loop structure, a pseudoknot, a bipartite nucleic acid duplex, a multi-partite nucleic acid triplex and a multi-partite nucleic acid tetraplex.

16. The polynucleotide probe of claim 15, wherein said predetermined secondary or tertiary structure is selected from sequences substantially related to: a catalytically active hairpin ribozyme, a catalytically inactive hairpin ribozyme, a truncated hairpin ribozyme, a tRNA, and a region from a ribosomal RNA.

17. The polynucleotide probe of claim 14, wherein said binding enhancer domain comprises a first subdomain located 5′ of said target binding domain and a second subdomain located 3′ of said target binding domain.

18. A set of polynucleotide probes comprising at least two polynucleotide probes according to claim 15, wherein each of said polynucleotide probes are specific for a different target polynucleotide.

19. The set of polynucleotide probes of claim 18, wherein each of said at least two polynucleotide probes comprises a unique identifier domain.

20. The set of polynucleotide probes of claim 18, wherein the binding enhancer domains of each of said at least two polynucleotide probes is the same.
